(19) United States
(12) Patent Application Publication
(10) Pub. No.: US 2024/0416037 A1
(43) Pub. Date: Dec. 19, 2024

(54) AUTO-INJECTOR AND RELATED METHODS OF USE

(71) Applicant: Regeneron Pharmaceuticals, Inc., Tarrytown, NY (US)

(72) Inventor: Bryan C. Grygus, Clifton Park, NY (US)

(73) Assignee: Regeneron Pharmaceuticals, Inc., Tarrytown, NY (US)

(21) Appl. No.: 18/744,010

(22) Filed: Jun. 14, 2024

Related U.S. Application Data
(60) Provisional application No. 63/551,995, filed on Feb. 9, 2024.
(60) Provisional application No. 63/508,780, filed on Jun. 16, 2023.

(51) Int. Cl. A61M 5/20; A61M 5/315

(52) U.S. Cl. CPC A61M 5/20; A61M 5/31511; A61M 2205/3368; A61M 2205/581

(57) ABSTRACT

An auto-injector, including a housing and a cartridge disposed within the housing, the cartridge enclosing a medicament. The auto-injector further includes a fluid conduit configured to deliver the medicament from the cartridge to a patient and movable from a retracted configuration to a deployed configuration associated with administering the medicament, and a temperature sensor configured to determine a temperature of the medicament. A controller the temperature sensor is configured to: send a first signal to an external device when the temperature sensor senses that a temperature of the medicament rises above a threshold temperature.

### CROSS-REFERENCE TO RELATED APPLICATION(S)

This patent application claims the benefit under 35 U.S.C. § 119 to U.S. Provisional Patent Application No. 63/508,780 filed on Jun. 16, 2023, and to U.S. Provisional Patent Application No. 63/551,995 filed on Feb. 9, 2024, the entireties of each of which are incorporated herein by reference.

### TECHNICAL FIELD

This disclosure is directed to an auto-injector and related methods of use.

### INTRODUCTION

In various available auto-injectors, upon activation by a user, a needle is deployed, and fluid is delivered from the needle into the user. After completion of fluid delivery, the needle may be retracted for user comfort, needle safety, and positive perception of the product. However, many auto-injectors lack adequate monitoring of system and patient data. Additionally, many auto-injectors may lack suitable control logic for stopping an injection when appropriate.

### BRIEF DESCRIPTION OF THE FIGURES

The accompanying drawings, which are incorporated in and constitute a part of this specification, illustrate various examples and together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 is a perspective view of an auto-injector, according to an example of the disclosure.

FIG. 1A is a perspective view of a portion of a housing of an auto-injector according to the disclosure.

FIG. 1B is a perspective view of a portion of a housing of an auto-injector according to the disclosure.

FIG. 2 is a bottom view of an auto-injector according to the disclosure.

FIG. 3 is a side view of an auto-injector, showing an activating switch extending away from a tissue-facing surface, according to the disclosure.

FIG. 3A is a cross-sectional view of an auto-injector, showing an activating switch extending away from a tissue-facing surface, according to the disclosure.

FIG. 3B is a cross-sectional view of an auto-injector, showing an activating switch in a partially depressed position, according to the disclosure.

FIG. 3C is a cross-sectional view of an auto-injector, showing an activating switch in a fully depressed position, according to the disclosure.

FIG. 4 is an exploded view of an auto-injector, according to the disclosure.

FIG. 4A is a schematic illustration of a control system of an auto-injector according to the disclosure.

FIG. 4B is an exploded view of an auto-injector according to the disclosure.

FIG. 4C is a perspective view of a portion of a housing and an electronics board, according to an aspect of the disclosure.

FIG. 5 is an exploded view of a needle mechanism according to the disclosure.

FIG. 5A is a perspective view of a fluid conduit according to the disclosure.

FIG. 5B is a cross-sectional view of a needle of a fluid conduit according to the disclosure.

FIG. 6 is a perspective view of the needle mechanism of FIG. 5 in a first position according to the disclosure.

FIGS. 7-11 are side views of the needle mechanism of FIG. 5.

FIG. 12 is a side cross-sectional view of a portion of an auto-injector according to the disclosure.

FIG. 13A is a side cross-sectional view of a piercing mechanism according to the disclosure.

FIG. 13B is a side cross-sectional view of an auto-injector according to the disclosure.

FIG. 14 is a side cross-sectional view of a piercing mechanism according to the disclosure.

FIG. 15 is a side view of a needle insert switch according to the disclosure.

FIG. 16A is a perspective view of a lock for an auto-injector according to an aspect of the disclosure.

FIG. 16B is a bottom view of an auto-injector and a lock according to the disclosure.

FIG. 16C is a cross-sectional view of an auto-injector and a lock according to the disclosure.

FIG. 16D is a cross-sectional view of an auto-injector and a lock according to the disclosure.

FIG. 16E is a perspective view of a lock for an auto-injector according to an aspect of the disclosure.

FIG. 16F is a side view of a lock for an auto-injector according to an aspect of the disclosure.

FIG. 17 is a bottom view of an electronics board for an auto-injector according to the disclosure.

FIG. 17A is a perspective view of an electronics board for an auto-injector according to the disclosure.

FIGS. 18-20 depict flowcharts of exemplary methods according to the disclosure.

FIGS. 20A and 20B depict graphs relating to electric controls for an auto-injector according to the disclosure.

FIGS. 21-23 depict flowcharts of exemplary methods according to the disclosure.

FIG. 24 depicts a diagram of patient data transmission according to the disclosure.

FIG. 25 depicts a functional block diagram of an auto-injector and a mobile device according to the disclosure.

FIG. 26 depicts different types of data associated with an auto-injector according to the disclosure.

FIG. 27 depicts an exemplary method for collecting biometric information according to the disclosure.

FIG. 28 depicts an exemplary method for verifying a patient's identity according to the disclosure.

FIG. 29 depicts an exemplary method for collecting data associated with the temperature of an injection site of the patient according to the disclosure.

FIG. 30 depicts an exemplary method of notifying a user of excessive swelling at an injection site according to the disclosure.

FIG. 31 depicts an exemplary method of comparing injection force data of an auto-injector according to the disclosure.

FIG. 32 depicts an exemplary method of measuring needle depth data during injection according to the disclosure.

FIG. 33 depicts an exemplary method of comparing injection data associated with an auto-injector according to the disclosure.

FIG. 34 depicts an exemplary method of comparing injection frequency data according to the disclosure.

FIG. 35 depicts an exemplary method of comparing temperature data of a medicament according to the disclosure.

FIG. 36 depicts an exemplary method of warming a medicament according to the disclosure.

FIG. 37 depicts an exemplary method of comparing administration rate data associated with an auto-injector according to the disclosure.

FIG. 38 depicts an exemplary method of verifying patient compliance with instructions for use associated with an auto-injector according to the disclosure.

FIG. 39 depicts an exemplary method comparing charge state of a battery of an auto-injector according to the disclosure.

FIG. 40 depicts an exemplary embodiment of emitting audio feedback from an auto-injector according to the disclosure.

FIG. 41 depicts an exemplary method of detecting a leak in an auto-injector according to the disclosure.

FIG. 42 depicts an exemplary method of verifying the inventory history of an auto-injector or a medicament according to the disclosure.

FIG. 43 depicts an exemplary method of locating an auto-injector according to the disclosure.

FIG. 44 depicts an exemplary method for correcting an error state of an auto-injector according to the disclosure.

FIG. 45 shows an exemplary method for detecting whether an auto-injector has been tampered with according to the disclosure.

FIG. 46 shows an exemplary method of displaying data associated with data collected through an auto-injector according to the disclosure.

FIG. 47 shows an exemplary method of detecting the strength of an adhesive associated with an auto-injector according to the disclosure.

FIG. 48 shows an exemplary method of determining the medicament type and dose of the medicament in an auto-injector according to the disclosure.

FIG. 49 shows an exemplary method of incentivizing compliance with a medicament administration regimen according to the disclosure.

FIG. 50 shows an exemplary method of responding to a patient's mental state associated with administering the medicament with an auto-injector according to the disclosure.

FIG. 51 shows an exemplary heat element in an auto-injector according to the disclosure.

FIG. 52 shows an exemplary heat element in an auto injector according to the disclosure.

Again, there are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the cross-sectional views are not drawn to scale and should not be viewed as representing proportional relationships between different components. The cross-sectional views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

As described above, existing auto-injectors may be inadvertently triggered when dropped or vibrated. Additionally, existing auto-injectors may lack suitable control logic for stopping an injection when appropriate. These shortcomings may cause premature deployment of drugs, increase complexity of self-administration of drugs, introduce user errors, and cause user discomfort.

Accordingly, the present disclosure is directed to various embodiments of an injection device (e.g., auto-injector) for self-administration of drugs, or other therapeutic agents, by a user. Specifically, according to certain embodiments, a likelihood of inadvertent triggering of the auto-injector may be reduced and the auto-injector may further incorporate control logic which improves operation of the auto-injector and user experience.

Additional details of auto-injectors in accordance with the present disclosure can be found in PCT/US2018/031077 to Arnott, et al., filed on May 4, 2018, and published as WO 2018/204779 A1, the entirety of which is incorporated by reference herein. Additional details of vial piercing systems in accordance with the present disclosure can be found in U.S. Pat. No. 10,182,969, filed on Mar. 10, 2016, the entirety of which is incorporated by reference herein.

### Overall System

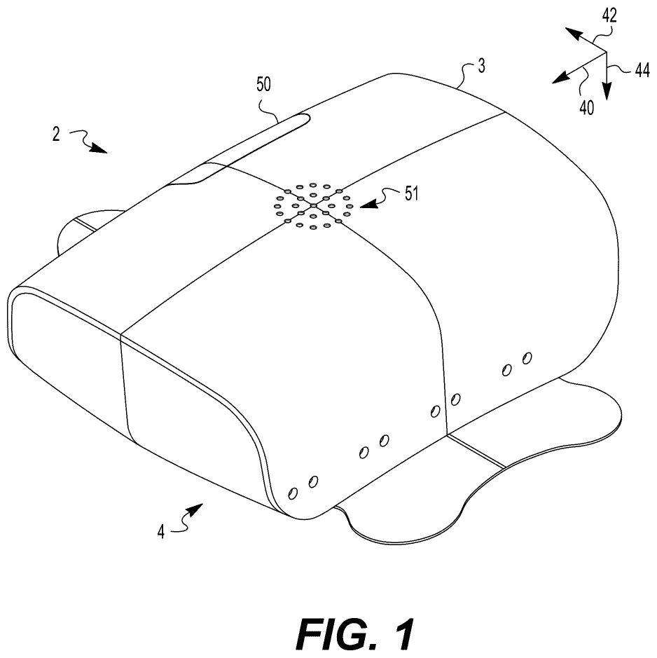

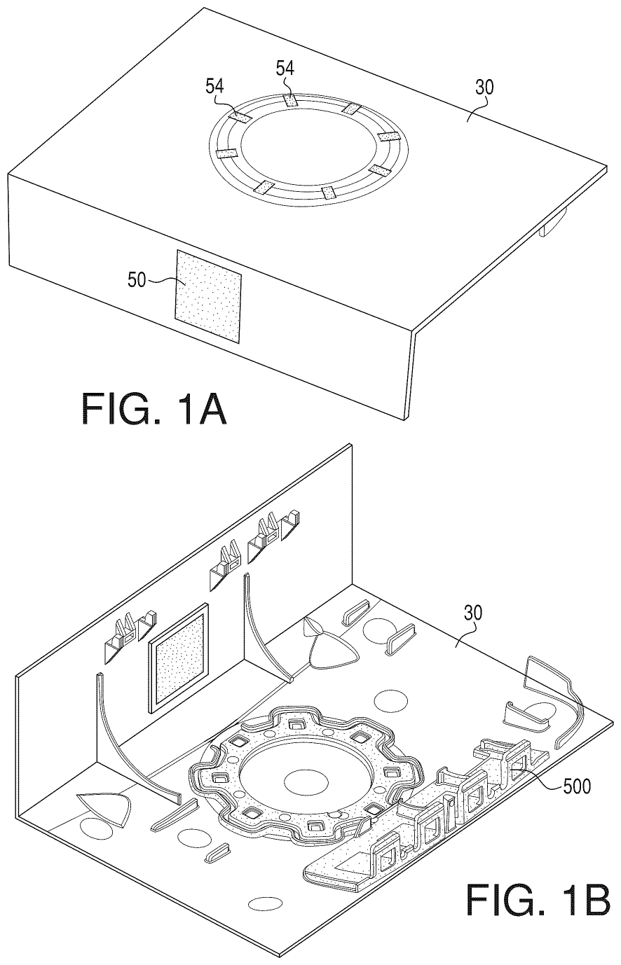

An example of such an auto-injector 2 is shown in FIGS. 1, 2, and 3. As shown in FIG. 1, auto-injector 2 may include a housing 3 having a tissue-engaging (e.g., bottom) surface 4 through which a needle may be deployed and retracted. As shown in FIGS. 1 and 1A, housing 3 may include a transparent window 50. Transparent window 50 may enable a viewer to visualize one or more displays or to visualize an interior of auto-injector 2 and components therein, such as a primary container and/or a drug product stored in the primary container.

In some embodiments, and as shown in FIG. 1, auto-injector 2 may include a plurality of openings 51 configured to facilitate the travel of sound generated within housing 3 (by, e.g., a speaker). Auto-injector 2 may have any suitable dimensions to enable portability and self-attachment by a user. In one example, auto-injector 2 may have a length of about 2.98 inches, a width of about 2.07 inches, and a height of about 1.07 inches. However, other suitable values also may be utilized, including, e.g., a length from about 0.5 inches to about 5.0 inches, a width of about 0.5 inches to about 3.0 inches, and a height from 0.5 inches to about 2.0 inches.

Auto-injector 2 may be oriented about a longitudinal axis 40 (e.g., an X axis), a lateral axis 42 (e.g., a Y axis) that is substantially perpendicular to longitudinal axis 40, and a vertical axis 44 (e.g., a Z axis) that is substantially perpendicular to both longitudinal axis 40 and lateral axis 42.

As shown in FIG. 1, an adhesive patch 12 may be coupled to tissue-engaging surface 4 to help secure auto-injector 2 to a user's body (e.g., skin). Adhesive patch 12 may be formed from fabric or any other suitable material, and may include an adhesive. The adhesive may be an aqueous or solvent-based adhesive, or may be a hot melt adhesive, for example. Suitable adhesives also include acrylic based, dextrin based, and urethane based adhesives as well as natural and synthetic elastomers. In some examples, the adhesive provided on patch 12 may be activated upon contact with a user's skin. In yet another example, patch 12 may include a non-woven polyester substrate and an acrylic or silicone adhesive. Patch 12 may be joined to housing 3 by, e.g., a double-sided adhesive, or by other mechanisms like ultrasonic welding. Patch 12 may have a length dimension greater than a width of auto-injector 2.

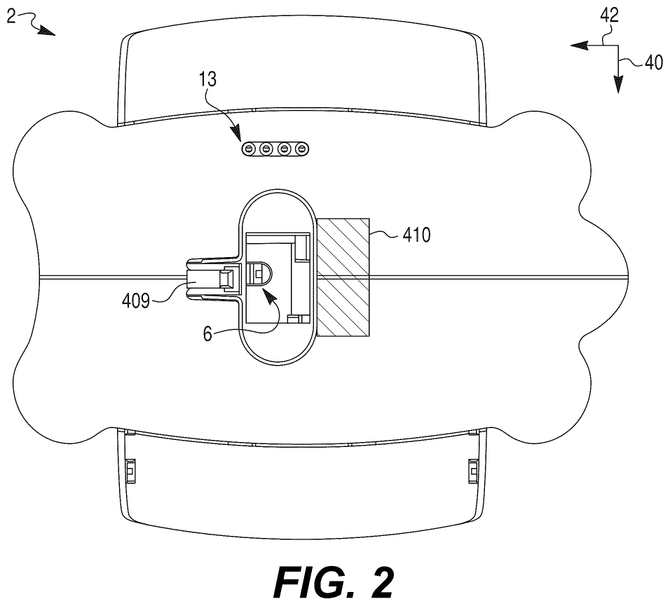

As shown in FIG. 2, auto-injector 2 may include an opening 6, through which the needle may be deployed and retracted. An activating switch 1409 may be disposed on tissue-engaging surface 4, and may be configured to activate auto-injector 2, or otherwise place auto-injector 2 in a “ready” mode. A touch sensor 1410 also may be disposed on tissue-engaging surface 4, and may be configured to help a controller of auto-injector 2 determine whether auto-injector 2 is disposed on the skin of a user (indicating that the auto-injector should fire or otherwise deploy a needle), or whether activating switch 1409 was improperly triggered (indicating that operation of auto-injector 2 should be stopped). A connecting port 13 also may be disposed on tissue-engaging surface 4 to facilitate programming of auto-injector 2.

Auto-injector 2 may be configured to operate in three or more operation phases including, e.g., an injection sequence activation phase, an injection phase, and a retraction phase, each of which will be described in further detail herein. The injection sequence activation phase, injection phase, and retraction phase may collectively be referred to herein as an “injection sequence.”

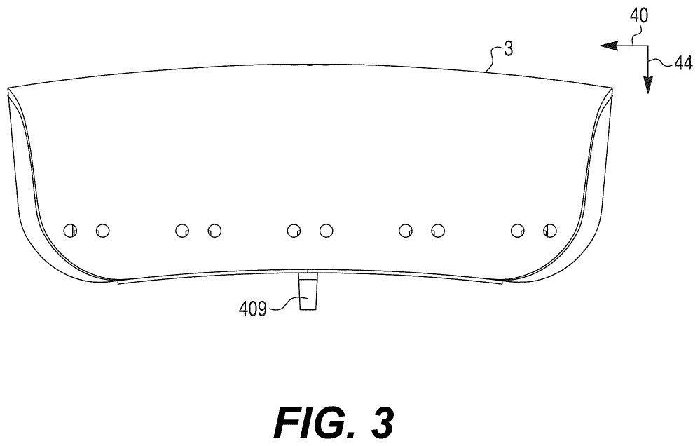

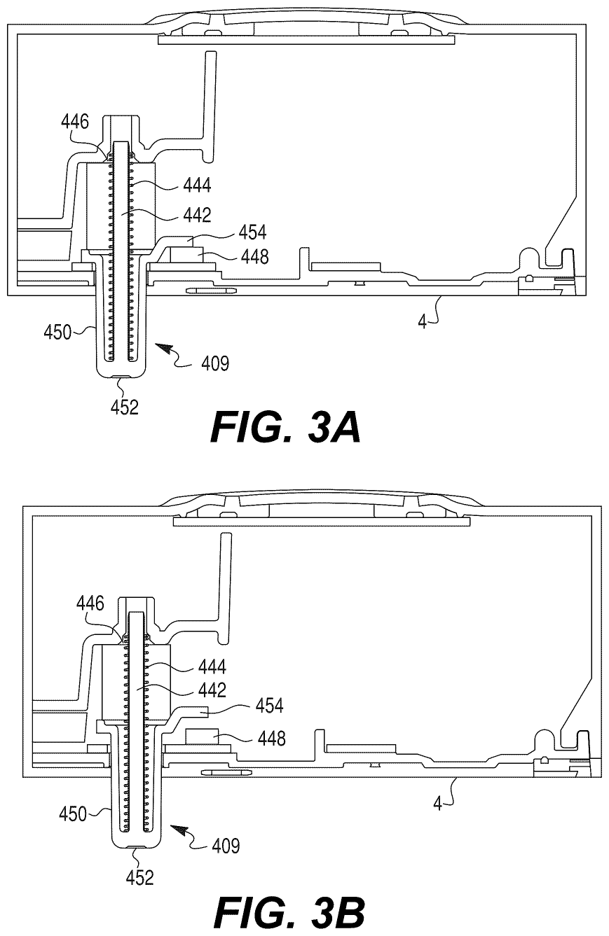

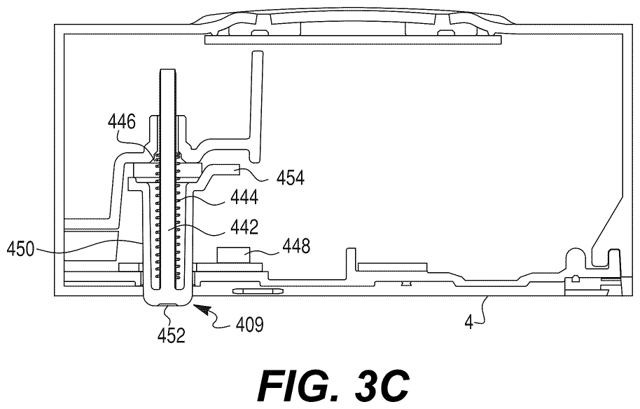

Referring to FIGS. 3A, 3B, and 3C which show cross-sections of an auto-injector 2, activating switch 1409 may be a mechanical plunger-type switch. For example, activating switch 1409 may include a plunger 1450 having a plunger contact surface 1452. Plunger contact surface 1452 may be generally circular in shape (or have another suitable shape) and may be large enough to be depressed comfortably by soft skin. In some embodiments, plunger contact surface 1452 may have a diameter or width ranging from about 2 mm to about 10 mm, a diameter ranging from about 4 mm to about 8 mm, or a diameter of about 6 mm. Activating switch 1409 may further include a shaft 1442, a biasing member 1444, a biasing collar 1446, and a plunger flange 1454. Biasing member 1444 may be a spring, for example, and may surround shaft 1442. Biasing member 1444 may be fixed, or otherwise prevented from moving, at one end by biasing collar 1446. Plunger flange 1454 may be configured to contact or otherwise depress a plunger switch 1448. For clarity, the term activating switch and/or reference to activating switch 1409, as used herein, should be understood to encompass any or all components of activating switch 1409, including shaft 1442, biasing member 1444, biasing collar 1446, plunger switch 1448, plunger 1450, plunger contact surface 1452, and plunger flange 1454.

In a free state, i.e., when plunger 1450 is not depressed, either by being pressed against the skin of a user or otherwise, plunger 1450 may extend outwardly from tissue-engaging surface 4 as shown in FIG. 3A. In the free state, plunger contact surface 1452 may be a distance from tissue-engaging surface 4 ranging from about 1 mm to about 16 mm, ranging from about 5 mm to about 12 mm, or a distance of about 8.5 mm. In the free state, biasing member 1444 may urge plunger 1450 to extend outwardly from tissue-engaging surface by pressing against biasing collar 1446. In the free state, plunger flange 1454 may be in contact with, or otherwise depress plunger switch 1448. When plunger flange 1454 is in contact with, or otherwise depresses plunger switch 1448, an electrical circuit associated with plunger switch 1448 may be complete, or closed.

When plunger 1450 is depressed either by being pressed against the skin of a user or otherwise, plunger 1450 may initially move to a partially depressed state, as shown in FIG. 3B. In the partially depressed state, biasing member 1444 may be compressed against biasing collar 1446. Plunger flange 1454 may further be out of contact with, or otherwise not depressing plunger switch 1448. When plunger flange 1454 is spaced apart from, not in contact with, or is otherwise not depressing plunger switch 1448, the electrical circuit associated with plunger switch 1448 may be broken, or open. By this configuration, the auto-injector 2 may be maintained in a reduced power state while plunger 1450 is depressed, such as when auto-injector 2 is in packaging.

As shown in FIG. 3B, plunger 1450 may not necessarily travel to the fully depressed state (shown in FIG. 3C) before plunger flange 1454 is out of contact with plunger switch 1448. As shown in FIG. 3C, on the other hand, in the fully depressed state, plunger 1450 may be depressed inwardly such that plunger contact surface 1452 is flush or nearly flush with tissue-engaging surface 4.

Plunger flange 1454 may be out of contact with plunger switch 1448, for example, after less than 5 mm of travel by plunger 1450, after less than 3 mm of travel by plunger 1450, after less than 1 mm of travel by plunger 1450, or after about. 75 mm of travel by plunger 1450—all when, for example, the maximum depression distance is 8.5 mm. In other words, plunger 1450 may transition from the free state, in which plunger flange 1454 is in contact with plunger switch 1448, to the partially depressed state, in which plunger flange 1454 is out of contact with plunger switch 1448, after moving only a portion of a maximum depression distance of plunger 1450 relative to housing 3 of auto-injector 2. For example, plunger 1450 may transition to the depressed state after moving only about 5%, about 10%, or about 20% of the maximum depression distance. Accordingly, the auto-injector 2 and plunger switch 1448 may be sufficiently responsive upon depressing plunger 1450 against a user's skin. For example, auto-injector 2 and plunger switch 1448 may be sufficiently responsive when pressed against skin of varying firmness or users having varying body fat content. While examples of travel distances for plunger 1450 are provided herein, it is to be understood that the present disclosure is not limited to any particular examples and any suitable travel distance may be used.

Biasing member 1444 may be sufficiently stiff such that in the free state, plunger flange 1454 stays in contact with or otherwise continuously depresses plunger switch 1448. Biasing member 1444 may also be of a stiffness such that plunger 1450 may be depressed comfortably when pressed against a user's skin. Biasing member 1444 may be biased to maintain plunger 1450 in the free state.

Though activating switch 1409 is shown in FIGS. 3A-3C as a mechanical plunger-type switch, it is to be understood that activating switch 1409 may be any other suitable type of switch, such as a rocker switch, optical switch, throw switch, toggle switch, temperature switch, and the like. Additionally, while the electrical circuit associated with plunger switch 1448 is described herein as closed when plunger 1450 is in the free state and open when plunger 1450 is in the depressed state, it should be understood that an opposite configuration may be used. For example, the electrical circuit associated with plunger switch 1448 may be open when plunger 1450 is in the free state and closed when plunger 1450 is in the depressed state. Additionally, or alternatively, activating switch 1409 may take the form of or include an integrated switch. For example, the plunger may include a conductive portion that closes a circuit when the conductive portion contacts the circuit board (e.g., first electronic circuit board 1402) and/or contacts electrical contacts mounted to or on the circuit board.

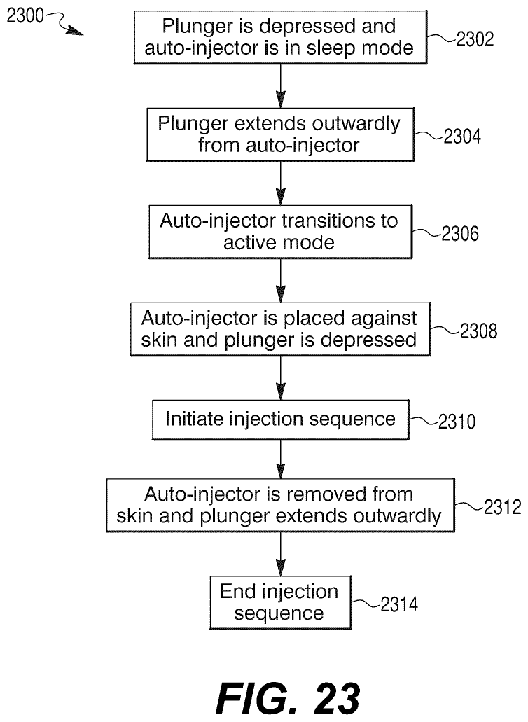

A method of controlling auto-injector 2 according to positions of activating switch 1409 will be described hereinafter in further detail with reference to FIG. 23.

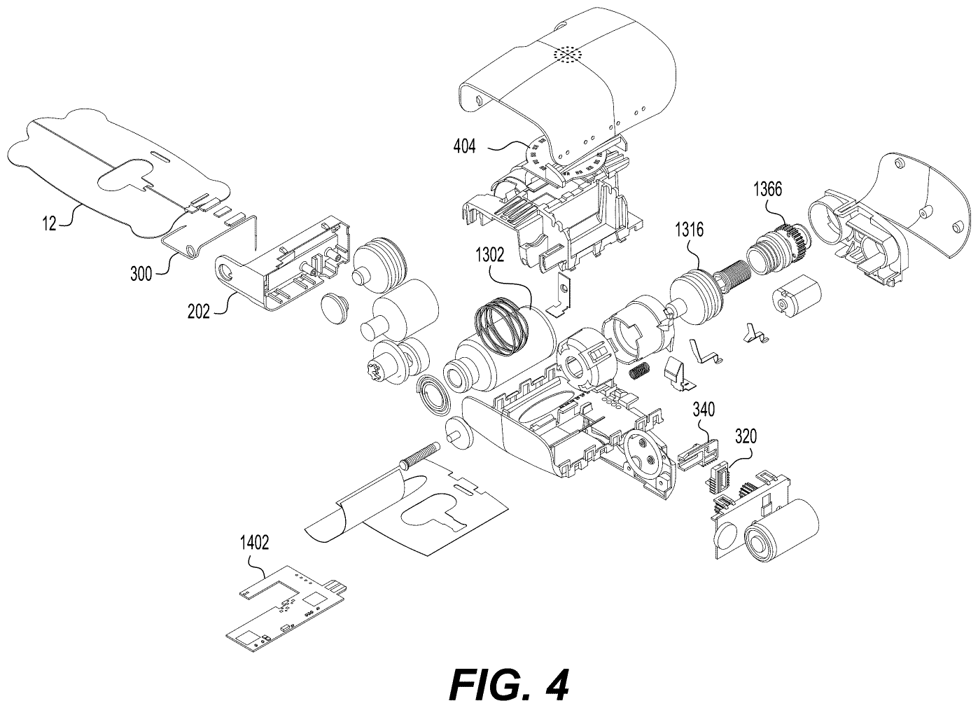

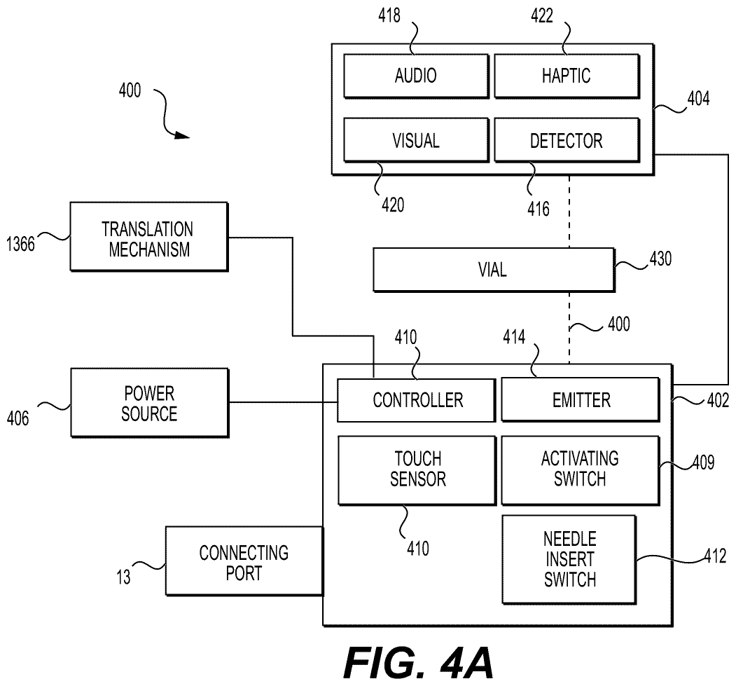

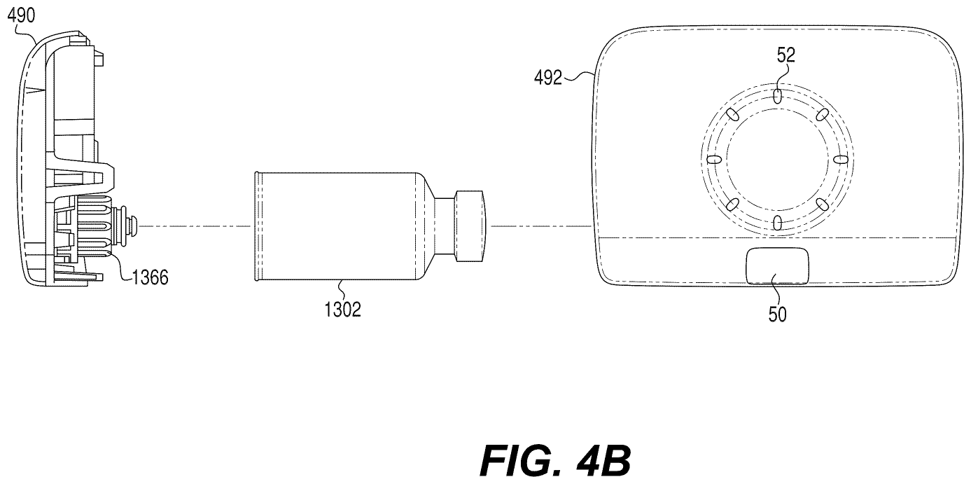

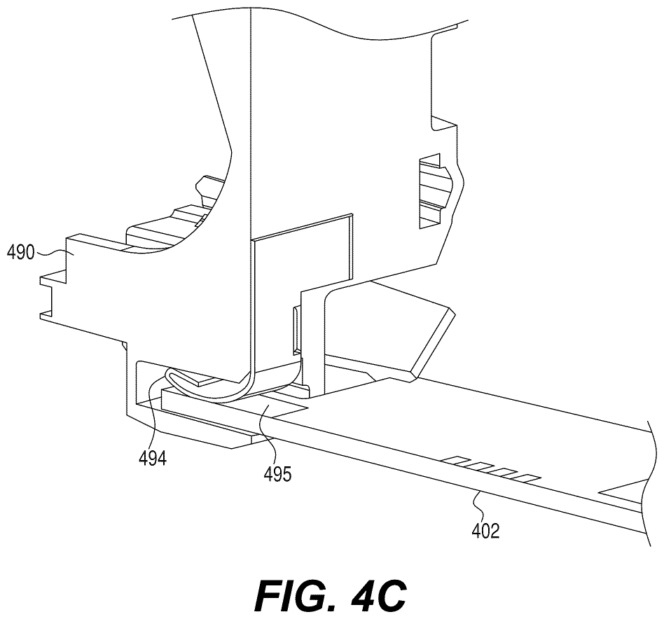

Further, as shown in FIG. 4B, in some embodiments, auto-injector 2 may include a plurality of LEDs 52. The LEDs 52 may be arranged in a ring-like formation, or any other suitable formation. As described in further detail hereinafter, light from the one or more LEDs 52 may be indicative of various operational states of the auto-injector 2.

### Auto-Injector Housing

Referring to FIGS. 1A and 1B, the housing 3 of auto-injector 2 may include an upper portion 30. The upper portion 30 may form a portion of the housing 3 opposite tissue-engaging surface 4. The upper portion 30 may include transparent window 50 through which a user may be able to see contents of the auto-injector 2, including a vial and/or a drug contained in the vial. The transparent window 50 may be positioned on a side of the upper portion 30 and may be formed such that it conforms to a rounded/curved contour of the upper portion 30, as shown in FIG. 1. The transparent window 50 may be generally rectangular in shape with rounded corners.

The upper portion 30 may also include a plurality of transparent windows 54. The transparent windows 54 may be formed on a top surface of upper portion 30 and may be arranged in any suitable configuration, such as a circular configuration, an oval configuration, a rectangular configuration, or a linear configuration, for example. Transparent windows 54 may be circumferentially spaced apart from one another, for example. The transparent windows 54 may allow light from one or more LEDs located within the housing 3 to be visible to a user. The light from the one or more LEDs may be indicative of various operational states of the auto-injector 2, as described herein.

The transparent window 50 and the transparent windows 54 may be integrally formed as part of the upper portion 30. As shown in FIG. 1B, the upper portion 30 may include a transparent portion 500 formed from a transparent material. The transparent portion 500 may be contiguous, such that the transparent window 50 and the transparent windows 54 are formed of one piece of transparent material. Moreover, the transparent portion 500 may be integrated into the upper portion 30 such that the upper portion 30, including the transparent window 50 and the transparent windows 54 is manufactured as a single part.

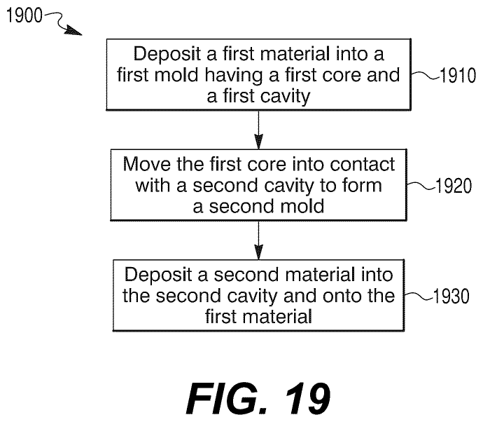

To form the upper portion 30 as a single part, the upper portion 30 may be manufactured using a double shot molding process, for example. FIG. 19 illustrates an exemplary method 1900 of molding the upper portion 30 using double shot molding or insert molding. At step 1910, a first material may be deposited into a first mold having a first core and a first mold cavity. The first material may have a low opacity and may be, for example, a transparent material for the transparent window 50 and the transparent windows 54. The first material may be, for example, transparent acrylic, clarified acrylonitrile butadiene styrene (ABS), polycarbonate, polyvinylchloride (PVC), or polyethylene terephthalate glycol (PETG). The first mold may be configured, for example, to form transparent portion 500.

At step 1920, the first core and the material in the first mold cavity, may be moved within a second mold cavity to form a second mold. When moved, the first core may retain the first material. The second mold may be configured, for example, to form the upper portion 30. At step 1930, a second material may be deposited into the second mold cavity in which the first material is contained. The second material may be deposited around the first material and first core in unoccupied space of the second mold cavity to form the upper portion 30. The second material may be a material with high opacity, such as white plastic. The second material may be, for example, ABS, polycarbonate, ABS-polycarbonate blend, PVC, or PETG.

Accordingly, the generally opaque upper portion 30, which includes the transparent window 50 and the transparent windows 54 may be formed from two different materials to form a single part. Forming the upper portion 30 as a single part may reduce an overall number of steps required to assemble auto-injector 2. For example, in some embodiments, no fastening or adhesive steps or materials are needed to join transparent and opaque portions of the housing. Avoiding unnecessary assembly steps may further improve the appearance of cosmetic surfaces of the auto-injector 2. Additionally, forming the upper portion 30 as a single part may improve the overall structural integrity of the auto-injector 2. Further, forming the upper portion 30 as a single part may reduce or eliminate sinks on cosmetic surfaces.

### Needle Mechanism

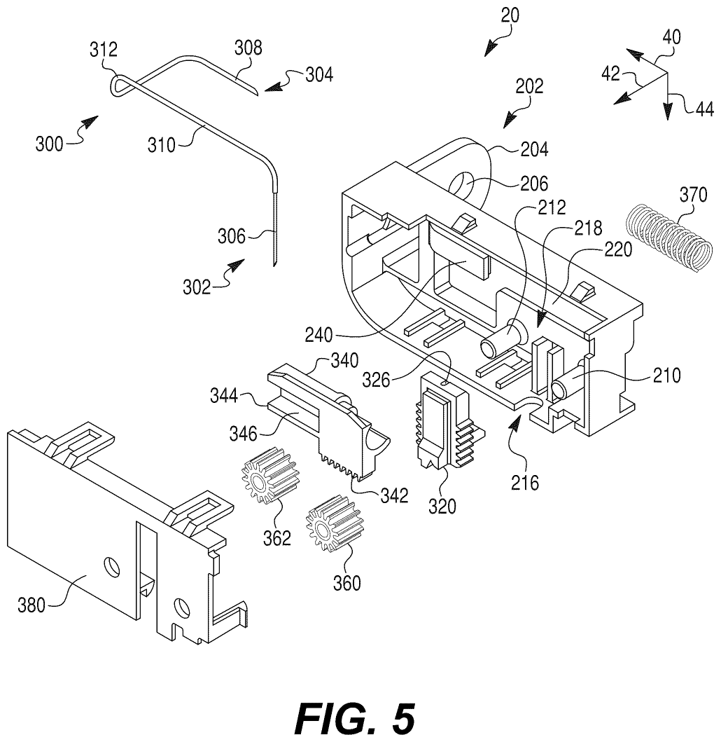

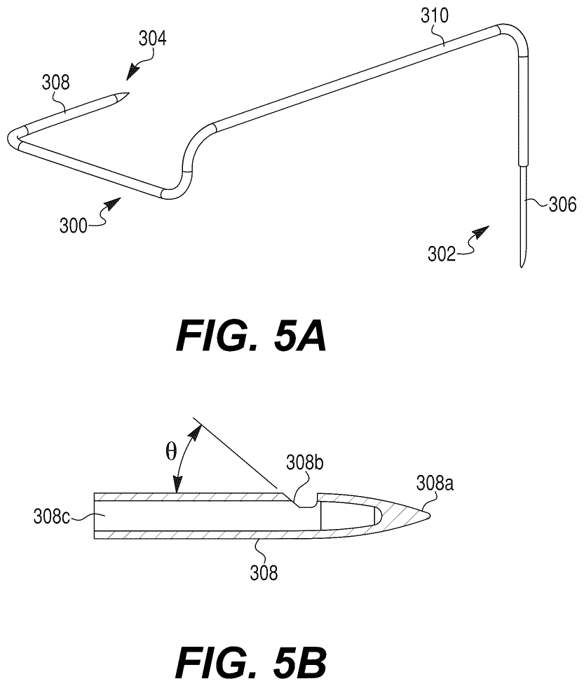

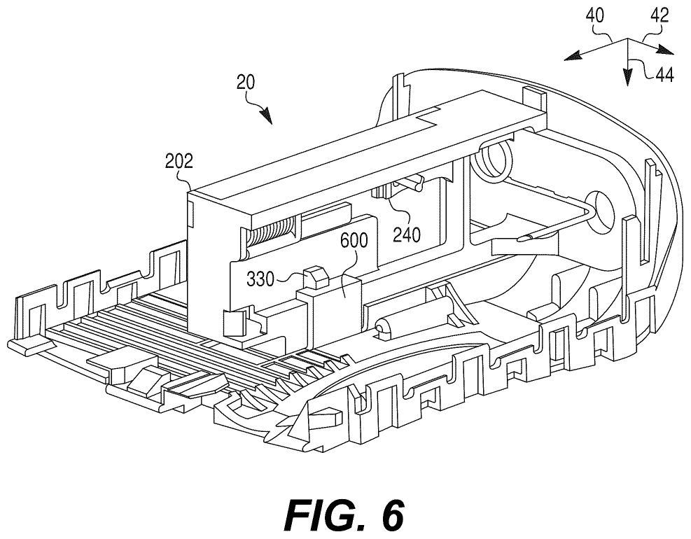

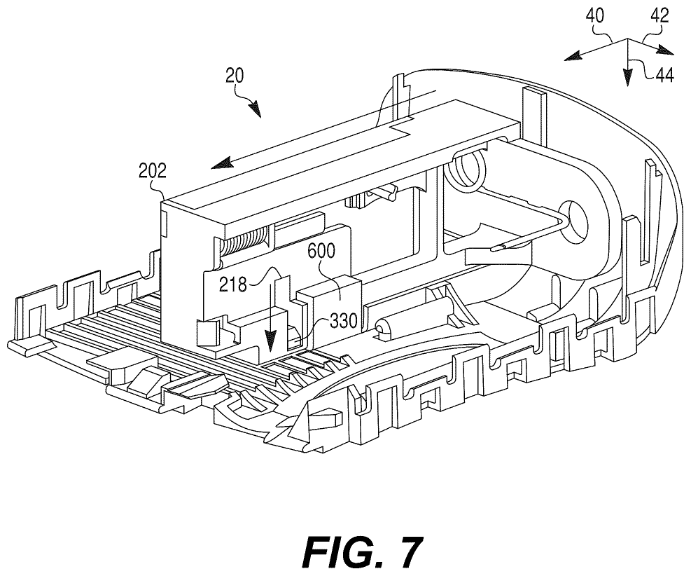

Referring to FIGS. 5-11, a needle mechanism 20 includes a carrier 202 that is movable (e.g., slidable) within housing 3 between a first position (FIG. 6) and a second position (FIG. 7). Needle mechanism 20 also may include a fluid conduit 300 that is mounted to carrier 202, and which may be deployed into a user, and retracted by a driver 320. A shuttle 340 (e.g., a shuttle actuator) may be configured to move driver 320 via a deployment gear 360, and a retraction gear 362. Shuttle 340 may be coupled to a resilient member (e.g., a spring 370). A cover 380 (FIG. 5) may be coupled to carrier 202 to enclose various components of needle mechanism 20.

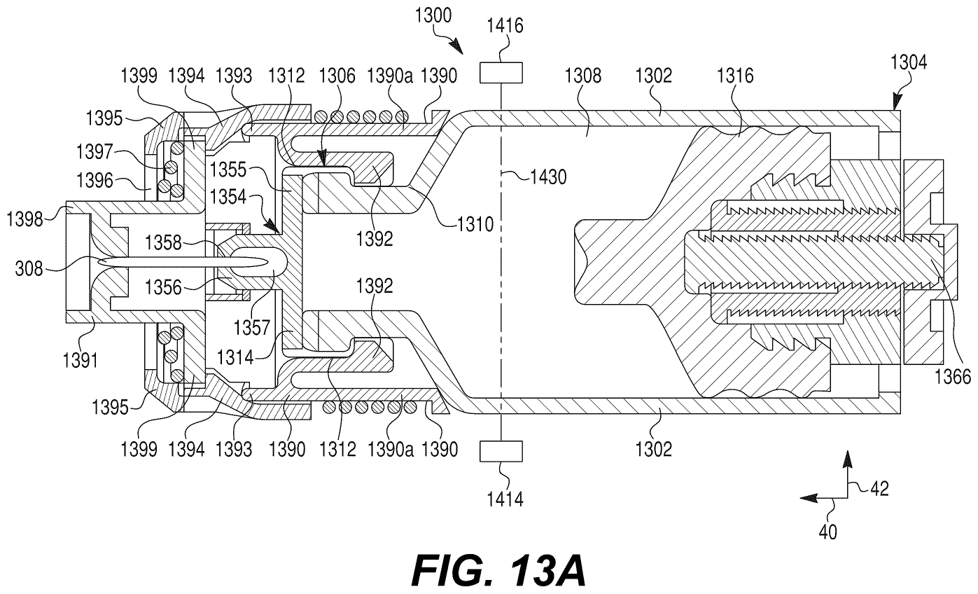

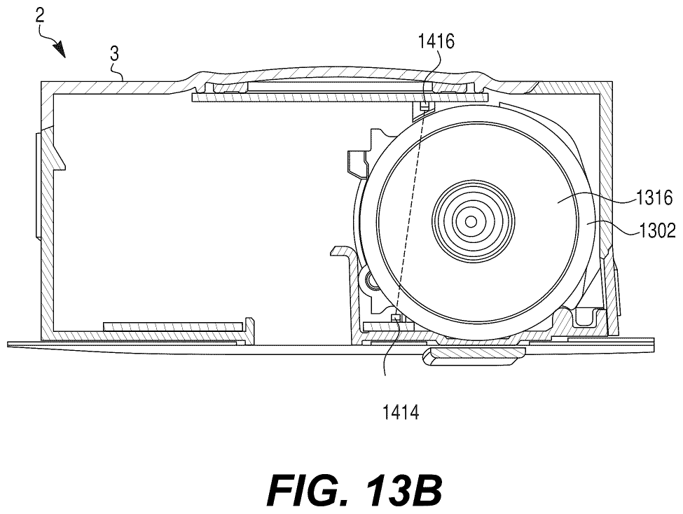

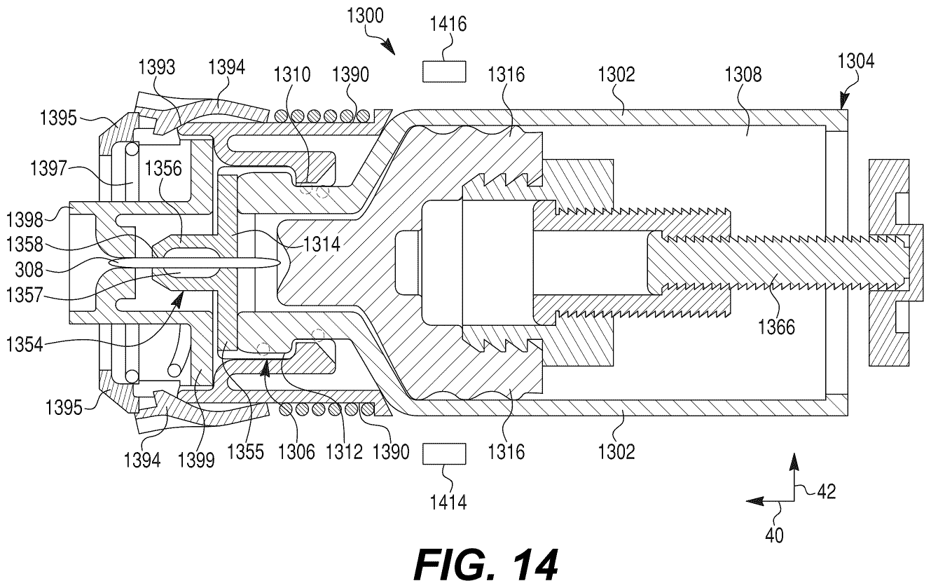

Referring to FIG. 5, fluid conduit 300 may extend from a first end 302 to a second end 304. As shown in further detail in FIG. 5A, first end 302 may include a needle 306 that is configured to be injected into a user. Needle 306 may include a sharp and/or beveled tip, and may extend generally along or parallel to axis 44. Second end 304 may include a needle 308 that is substantially similar to needle 306, but may be positioned within auto-injector 2 to penetrate a cartridge 1302 (shown in FIG. 13A and described in further detail below) to access drugs to be injected into the user. Fluid conduit 300 may include an intermediate section 310 including one portion extending along or parallel to axis 40, and a second portion extending along or parallel to axis 40. The first and second portions of intermediate section 310 may be joined in a serpentine section 312 that facilitates flexion of fluid conduit 300 and movement of needle 306 along axis 44 during deployment into the user, and during retraction out of the user. While a serpentine section 312 is shown, any other suitable shape, e.g., a coil, curved, or other shape that enables flexion of fluid conduit 300 is also contemplated. Serpentine section 312, or similar structure, may act as a cantilever when needle 306 is deployed and/or retracted. Serpentine section 312 also may bias fluid conduit 300 into the deployed configuration shown in FIG. 5. Once needle 308 penetrates and establishes fluid communication with cartridge 1302 (see, e.g., FIG. 14), drugs may travel from cartridge 1302, through needle 308, intermediate section 310, and needle 306 (pierced through the user's skin), and into the user. In some examples, fluid conduit 300 may include only metal or a metal alloy. In other examples, fluid conduit 300 may be any other suitable material, such as, e.g., polymers or the like. Needle 308 and intermediate portion 310 may define a 22 or 23 Gauge, thin-walled needle, while needle 306 may be a 27 Gauge, thin-walled needle. Other needle sizes ranging from, e.g., 6 Gauge to 34 Gauge, and other needle wall thicknesses, such as regular wall, extra-thin wall, and ultra-thin wall also may be utilized as appropriate. Fluid conduit 300 may reduce the amount of material that contacts the drugs, reduce joints and assembly steps, and require less sterilization than conventional devices.

As shown in FIG. 5B, needle 308 may be configured to include a needle tip 308a and a side port 308b. Side port 308b may be fluidly connected to fluid path 308c and allow fluid to enter fluid conduit 300 through a side of needle 308, as opposed to through the tip of needle 308. A rear wall of side port 308b may be inclined at an angle θ relative to a longitudinal axis of fluid path 308c. In some embodiments, the angle θ may be between about 20° and 60°, between about 30° and 50°, or about 40°. By configuring needle 308 in this way, needle 308 may be optimized for piercing the primary container of auto-injector 2, which may be a sealed cartridge or a vial. The relative positioning of needle tip 308a and side port 308b may allow piercing of the seal of the primary container without coring or otherwise cutting a portion of the seal with an opening to fluid path 308c. Thereby, entry of particles cored or cut from the seal into the fluid path 308c may be minimized or avoided.

Needle 306 may be configured substantially similarly to needle 308, as shown in FIG. 5B. Alternatively, in some embodiments, either or both of needles 306 and 308 may be a 3 bevel needle, a 5 bevel needle, or any other suitable type of needle. In some embodiments, one or both of needles 306 and 308 may be a pencil point needle having a round hole or any other suitably shaped hole.

Carrier 202 may be formed of plastic (e.g., injection-molded plastic), a metal, metal alloy, or the like, and may include a flange 204 with an opening 206, and posts 210 and 212. Carrier 202 also may include an opening 216 through which a needle or other fluid conduit may be deployed. Opening 216 may be a slot that is recessed from an end surface of carrier 202, or, in an alternative embodiment, an entirety of the perimeter of opening 216 may be defined by material of carrier 202. Carrier 202 also includes a driver path 218. Driver path 218 may be a slot in carrier 202 that extends along or parallel to axis 44. Driver path 218 may be configured to receive a protrusion of driver 320, such as, e.g., protrusion 330 discussed in further detail below. Carrier 202 also may include a shuttle path 220, along which shuttle 340 may move, as described in further detail below.

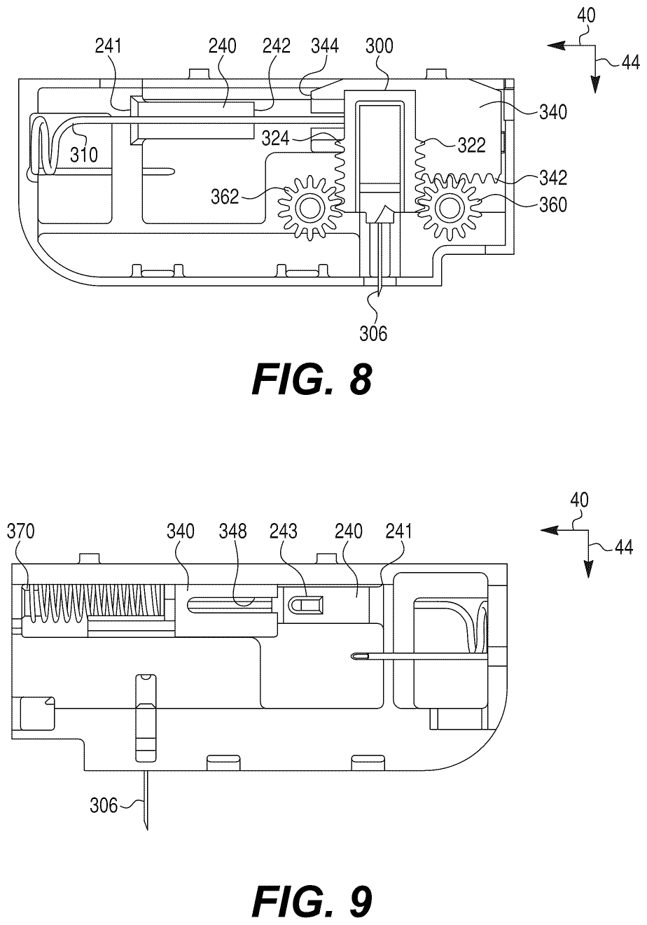

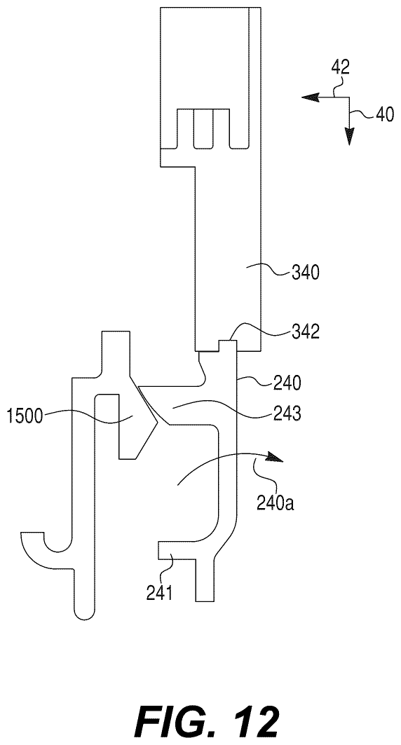

Carrier 202 also may include a stop 240 that is configured to engage shuttle 340. Stop 240 may be a cantilever having a fixed end 241 (FIG. 8) and a free end 242 (FIG. 8). Stop 240 may include an inclined ramp 243 (FIGS. 9 and 12) that, when engaged or pushed by a ramp 1500 (described with reference to FIG. 12), causes stop 240 to deflect about fixed end 241. In a first position, free end 242 may block or otherwise impede movement of shuttle 340, and in a second configuration, may permit movement of shuttle 340. The relationship between stop 240 and shuttle 340 will be discussed in further detail later in the application.

Driver 320 includes two racks 322 and 324 (shown in FIG. 8) parallel to one another and disposed on opposing sides of driver 320. Racks 322 and 324 may include teeth and may be configured to engage with and drive rotation of deployment gear 360 and retraction gear 362, respectively. Driver 320 may include a lumen 326 (or a track, recess, or other suitable structure) (FIG. 5) that is configured to receive needle 306 of fluid conduit 300. Driver 320 also may include protrusion 330 (FIGS. 6 and 7) that is configured to slide within driver path 218 of carrier 202. Protrusion 330 may include a hook-like configuration that can “catch” on impediment 600, as described in further detail below.

With continuing reference to FIG. 5, shuttle 340 may include a rack 342 configured to engage with gears 360 and 362. Shuttle 340 also may include an end surface 344, and a recess 346 that extends along a length of shuttle 340 in the same direction as rack 342. A slot 348 (FIG. 9) may extend along the length of recess 346. Slot 348 may extend through the middle of recess 346 and may extend along an entirety or substantial entirety of recess 346.

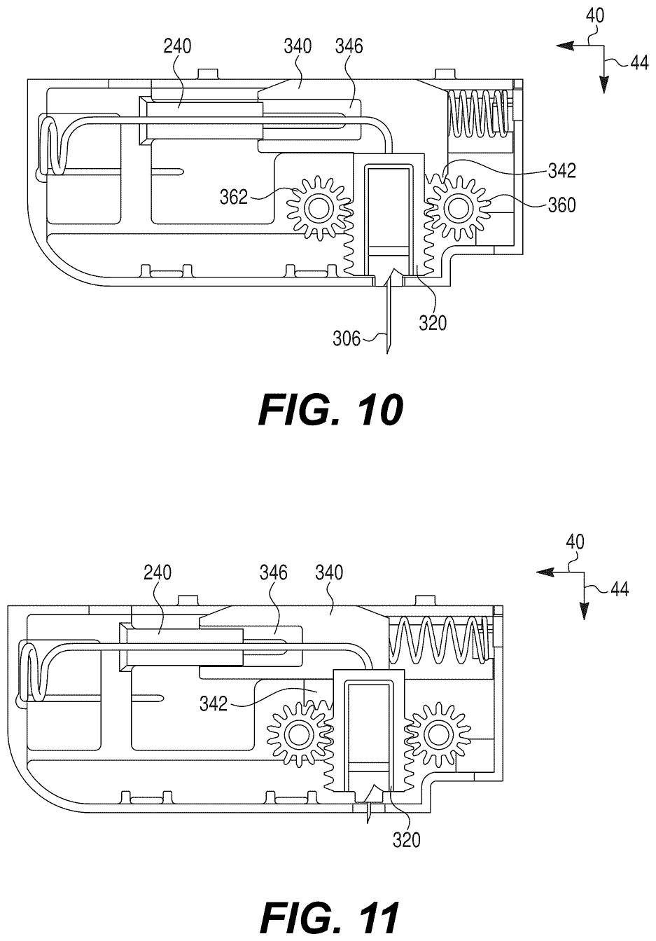

Shuttle 340 may move along track 220 from a first, starting position (FIG. 8), to a second, intermediate position (FIGS. 9 and 10), and from the second position to a third, final position (shown between the second and third configurations in FIG. 11). As shuttle 340 moves along track 220, rack 342 may first engage deployment gear 360, and then retraction gear 362. At certain times, rack 342 engages at most one of deployment gear 360 and retraction gear 362 at any given time. In some examples, such as when rack 342 is disposed longitudinally between deployment gear 360 and retraction gear 362, rack 342 is not engaged with either of deployment gear 360 and retraction gear 362. Shuttle 340 may be configured to move only along one axis (e.g., axis 40) and only in one direction along the one axis. The force required to move shuttle 340 along track 220 may be provided by expansion of spring 370. Spring 370 may be compressed from a resting state, and the expansion of spring 370 may move shuttle 340 along track 220 through the series of positions/configurations set forth above. At various positions of shuttle 340, different features of auto-injector 2 may directly or indirectly block movement of shuttle 340. Alternatively, it is contemplated that spring 370 may be biased to a compressed configuration. In this alternative embodiment, spring 370 may be expanded from a resting state, and compression of spring 370 may move shuttle 340 along track 220 through the series of positions/configurations set forth above.

The first position of shuttle 340, shown in FIG. 8, may correspond to an unused, undeployed, and/or new state of auto-injector 2. In this first position, driver 320 may be in an undeployed state. Shuttle 340 is maintained in the first position by the positioning of an impediment 600 in the path of driver 320 (FIG. 6). Impediment 600, which may be a shelf of housing 3, or another suitable blocking device, may prevent movement of driver 320 by engaging and/or retaining protrusion 330. Therefore, because driver 320, deployment gear 360, and rack 342 are coupled to one another, the blockage of driver 320 also prevents movement of shuttle 340. Shuttle 340 may move from the first position to the second position by moving impediment 600 relative to carrier 202 (or vice versa). In one example, carrier 202 is moved (e.g., to the left in FIG. 6) while impediment 600 remains stationary.

When the path of driver 320 is free from impediment 600 (FIG. 7), spring 370 may expand and move shuttle 340 along track 220. This linear movement of shuttle 340 may rotate deployment gear 360 counter-clockwise (or clockwise in other examples) via rack 342, and the rotation of deployment gear 360 may move driver 320 downward along axis 44, via rack 322 of driver 320. This downward movement of driver 320 may cause needle 306 to pierce through the skin of a user. In some examples, driver 320 may be configured to move, relative to carrier 202, along only axis 44.

Shuttle 340 may be moved by the expansion of spring 370 until its end surface 344 abuts free end 242 of stop 240 such that shuttle 340 is maintained in the second position shown in FIGS. 9 and 10. At this point, free end 242 may prevent further expansion of spring 370 and further movement of shuttle 340 along track 220. In this second position, fluid conduit 300 may be deployed within a user, and fluid from cartridge 1302 may be injected into the user via needle 306. Additionally, while shuttle 340 is in the second position, rack 342 may be engaged with deployment gear 360 to maintain needle 306 in the deployed configuration. Shuttle 340 may move from the second position to the third position by the flexion of stop 240 about its fixed end 241. Further details of this flexion are set forth below with respect to FIGS. 12-14. The flexion of stop 240 may allow spring 370 to continue expanding, urging shuttle 340 further along track 220. In some examples, stop 240 may be received by and/or within recess 346 of shuttle 340, and ramp 243 may slide within slot 348, as shuttle 340 moves from the second position to the third position.

The movement of shuttle 340 from the second position to the third position may correspond to the retraction of needle 306 from the user into housing 3. In particular, rack 342 may engage with and rotate retraction gear 362 in the same direction (e.g., counter-clockwise or clockwise) as deployment gear 360 was rotated. The rotation of retraction gear 362 may urge driver 320 back to a retracted position via rack 324. Shuttle 340 may reach the third position, where driver 320 is fully-retracted, when its end surface 344 engages a wall of carrier 202, when free end 242 of stop 240 reaches an end of recess 346, and/or when spring 370 reaches a resting state.

In some embodiments, once driver 320 moves from the deployed state back to the retracted state, it may be prevented from moving out of the retracted state. As a result, needle 306 will be prevented from re-deployment into the user. In this configuration, auto-injector 2 may be a single-use device (e.g., discarded after completing one injection). In other embodiments, auto-injector 2 may be reset and reused. Furthermore, deployment gear 360 and retraction gear 362 may be the only rotating gears disposed within auto-injector 2, in some examples.

### Piercing System and Sterile Connector

FIGS. 13 and 14 show features of a piercing system 1300 of auto-injector 2. Additional details of exemplary piercing systems can be found in U.S. Patent Application Publication No. 2016/0262984 A1 to Arnott et al., published on Sep. 15, 2016, the entirety of which is incorporated by reference herein. Piercing system 1300 includes a primary container, which may be a cartridge 1302 with a first end 1304 and a second end 1306. The primary container may alternatively be a chamber, syringe, vial, flexible sac, or any other suitable fluid containing structure.

Cartridge 1302 may include a cavity 1308 opened at first end 1304 and extending toward second end 1306. Second end 1306 may include a neck 1310 with a cap 1312 that engages neck 1310 to close second end 1306. A septum 1314 may be positioned between cartridge 1302 and cap 1312 to assist with closing second end 1306, and allow for needle 308 (e.g., a staked needle) to be inserted into cartridge 1302. Cavity 1308 may be closed at first end 1304 by a piston 1316.

Cartridge 1302 may have a 5 mL capacity in some examples, although any other suitable volume (e.g., from 1 mL to 50 mL, or from 2 mL to 10 mL, or from 3 mL to 6 mL, or from 2 mL to 5 mL, or another suitable range) also may be utilized depending on the drug to be delivered. In other examples, cartridge 1302 may have a capacity greater than or equal to 1 mL, or greater than or equal to 2 mL, or greater than or equal to 3 mL, or greater than or equal to 4 mL, or greater than or equal to 5 mL, or greater than or equal to 10 mL, or greater than or equal to 15 mL. Cartridge 1302 may contain and preserve a drug for injection into a user, and may help maintain sterility of the drug. Cartridge 1302 may have a 13 mm diameter neck, a 45 mm length, and an internal diameter of 19.05 mm. These values are merely exemplary, and other suitable dimensions may be utilized as appropriate. In some examples, cartridge 1302 may be formed using conventional materials, and may be shorter than existing devices, which can help auto-injector 2 remain cost-effective and small. Cartridge 1302 may be a shortened ISO 10 mL cartridge.

Septum 1314 may include an uncoated bromobutyl material, or another suitable material. Piston 1316 may include a fluoropolymer coated bromobutyl material, and also may include a conical nose 1316a to help reduce dead volume within cartridge 1302. Piston 1316 may include one or more rubber materials such as, e.g., halobutyls (e.g., bromobutyl, chlorobutyl, florobutyl) and/or nitriles, among other materials.

Piercing system 1300 also may include a top 1354 positioned at second end 1306. Top 1354 may include a base 1355 positioned over septum 1314 and the opening of cartridge 1302. Top 1354 may include a chamber 1356 extending from base 1355 in a direction away from piston 1316. Chamber 1356 defines a cavity 1357 and includes an opening 1358 in communication with cavity 1357. In some embodiments, top 1354 may be integrated with septum 1314 (e.g., integral or of one-piece construction). In alternative embodiments (not shown), top 1354 may be provided or initially assembled on fluid conduit 300 and not installed directly on/with cartridge 1302 and/or integrated with septum 1314.

A portion of fluid conduit 300, such as needle 308, a tube or the like, may extend through opening 1358 of chamber 1356 and into cavity 1357, but not through base 1355 in the pre-activated state. Opening 1358 may be pre-formed, or may be formed by the penetration of needle 308 through chamber 1356. Opening 1358 of chamber 1356 may form a sterile sliding seal about needle 308 such that pathogens or other contaminants are prevented from passing into cavity 1357. Needle 308 can move relative to top 1354 without disrupting the sterile seal therebetween. Cavity 1357 may be sterile or aseptic such that the inner surfaces of cavity 1357 and needle 308 are sterile. In another embodiment, cavity 1357 may be sterilized after needle 308 is inserted through opening 1358 and into cavity 1357. In alternative embodiments, rather than top 1354, a convoluted flexible (e.g., rubber) bellows or bladder member may form cavity 1357 and allow translation of cartridge 1302 relative to needle 308 (or vice versa). The flexible member also may seal or form cavity 1354 about needle 308 after sterilization.

Piston 1316 may be coupled to a translation mechanism 1366 that is configured to translate piston 1316 and cartridge 1302 in a direction toward second end 1306. The movement of piston 1316 toward second end 1306 causes piston 1316 to act against the contents within cartridge 1302 (e.g., drugs, medications), which ultimately transfers force against second end 1306 of cartridge 1302, causing cartridge 1302 to move along longitudinal axis 40. Translation mechanism 1366 may include a 12 mm motor with a five-stage gear reduction (360:1). Translation mechanism 1366 may have spring contacts that create an electrical connection with an associated printed circuit board (e.g., first electronic board 1402). The motor may be configured to generate a torque of about 136 mN*m at 36 rpm. These design parameters of the motor are merely exemplary, and any other suitable motor also may be utilized.

Translation mechanism 1366 may include a leadscrew mechanism coupled to piston 1316 that extends axially upon relative rotation about longitudinal axis 40. This telescoping leadscrew may have a 100 N output, a 20 mm stroke, and a 7°/45° buttress thread shape with a 0.75 mm pitch. The materials for the leadscrew mechanism may include acetal and polybutylene terephthalate. The leadscrew mechanism may extend within piston 1316 to reduce dead space behind piston 1316. While piston 1316 is shown in FIGS. 13A and 14 with longitudinally spaced threads, in some examples, such threads may not be present. In another exemplary embodiment (not shown), translation mechanism 1366 may include a manually engageable surface or member that is manually manipulated by a user to move piston 1316. For example, piercing system 1300 may include a cartridge or a plunger coupled to the back side of piston 1316. In another exemplary embodiment (not shown), translation mechanism 1366 may include a pneumatic or hydraulic drive member that is actuated or initiated by a user to move piston 1316. The drive member may be in the form of expanding bellows, an expanding bladder, an expanding diaphragm, or a sliding seal or piston, for example. The direct pneumatic or hydraulic pressure may provide the force required to move piston 1316.

Piercing system 1300 also includes a collar 1390 coupled or fixed to second end 1306. Collar 1390 may include a plurality of circumferentially spaced apart fingers 1392 that engage and surround neck 1310. Collar 1390 may be fixed, or otherwise coupled to second end 1306. Collar 1390 may include a wall 1390a that extends at least partially about neck 1310, the opening of second end 1306, cap 1312, septum 1314, and/or top 1354. Wall 1390a of collar 1390 may be positioned radially or laterally outward of neck 1310 and extend longitudinally past neck 1310, cap 1312, and septum 1314.

In the pre-activated state of piercing system 1300 shown in FIG. 13A, an edge 1393 of collar 1390 may engage a corresponding radially or laterally inwardly extending cam, latch or actuation portion 1394 of a driver retainer member 1395. Retainer member 1395 may be slidable relative to collar 1390. Collar 1390 and retainer member 1395 may be configured such that in the pre-activated state or arrangement shown in FIG. 13A, at least a portion of the cam or actuation portion 1394 of retainer member 1395 is positioned directly behind a retaining portion 1399 of a driver 1398 slidable within retainer member 1395. A wall 1391 of driver 1398 may extend into and through an end cap portion 1396 of retainer member 1395 and into an interior portion of retainer member 1395, and retaining portion 1399 of driver 1398 may extend radially outward from wall 1391. In some embodiments, wall 1391 of driver 1398 may be substantially cylindrical and retaining portion 1399 of driver 1398 may be a flange extending about an end of the wall 1391.

In the pre-activated state of piercing system 1300, an elastically deformed biasing or resilient member 1397 may be positioned between cap portion 1396 of retainer member 1395 and retaining portion 1399 of driver 1398. Biasing member 1397 may exert a force against driver 1398 in the pre-activated state of piercing system 1300 acting in the direction towards cartridge 1302. Biasing member 1397 may be any member effective in applying the force in the pre-activated state, and then releasing such force upon activation, as discussed below with reference to FIG. 14. In some embodiments, biasing member 1397 may be a conical or flat spring.

Needle 308 of fluid conduit 300 may be fixed or coupled to driver 1398 such that fluid conduit 300 moves with driver 1398. In the pre-activated state of piercing system 1300, needle 308 may be positioned within the sterile cavity 1357, but not through base 1355 of top 1354, septum 1314, and/or into cavity 1308 of cartridge 1302.

In some embodiments, in lieu of cavity 1357, needle 308 may be positioned within a plug when the piercing system 1300 is the pre-activated state. The plug may be a solid plug which is devoid of any holes, cavities, or openings, and which may be formed of a first rubber material. The first rubber material may be permeable to a sterilizing gas, such as, e.g., ethylene oxide or vaporized hydrogen peroxide. The first rubber material may include one or more of isoprene, ethylene propylene diene monomer (M-class) rubber (EPDM), and styrene-butadiene, among others. The permeability of the first rubber material to a sterilizing gas may allow needle 308, when disposed within the plug, to be sterilized before use. The plug may be molded about needle 308, so that needle 308 is impaled into the plug.

To move piercing system 1300 from the pre-activated state of FIG. 13A, translation mechanism 1366 may be activated to move piston 1316 towards second end 1306 and translate cartridge 1302 along longitudinal axis 40 toward driver 1398. Because the needle 308 is not yet in fluid communication with cartridge 1302, activation of translation mechanism 1366 applies a pressure against the fluid contained in cartridge 1302, which is then applied to cartridge 1302 itself. This pressure also causes edge 1393 to push against and deflect actuation portion 1394 radially outward. Without actuation portion 1394 blocking its path, retaining portion 1399 and needle 308 are moved toward cartridge 1302 by the expansion of biasing member 1397. Driver 1398 may be coupled to flange 204 of carrier 202, and thus, this movement of driver 1398 toward cartridge 1302 also may move carrier 202 in the same direction. This movement corresponds to the movement of carrier 202 relative to housing 3 in FIGS. 6 and 7, which enables protrusion 330 to clear impediment 600 to inject needle 306.

The movement of needle 308 toward second end 1306 of cartridge 1302 also causes needle 308 to pierce through base 1355 of top 1354, septum 1314, and cavity 1308, into fluid communication with the contents of cartridge 1302. Once needle 308 is in fluid communication with cartridge 1302, further movement of piston 1316 toward second end 1306 urges fluid through needle 308 and a remainder of fluid conduit 300. In some embodiments, piercing system 1300 may be configured such that, after activation, no more of needle 308 than the portion that was already positioned within sterile cavity 1357 extends into cavity 1308. This may help prevent contamination of the contents of cartridge 1302 with non-sterile portions of needle 308.

Biasing member 1397 may be configured to expand such that fluid conduit 300 pierces top 1354 and/or septum 1314 at a high speed, such as at a speed of at least about 10 mm/sec, or at least about 40 mm/sec. The relatively quick piercing of top 1354 and/or septum 1314 via biasing member 1397 may help prevent leakage of the contents of cavity 1308 which may be under pressure via piston 1316.

After drugs have been delivered to the user via needle 306, needle 306 may be automatically withdrawn from the user. Referring to FIGS. 12-14, translation mechanism 1366 may be operated in a reverse mode such that the rotation of the lead screw is in an opposite direction compared to the insertion step. This counter-rotation may cause piston 316 to move back toward first end 1304, and also cause cartridge 1302 to move in an opposite direction along axis 40 (as compared to during fluid delivery and insertion of needle 306). The movement of cartridge 1302 in the opposing direction may cause ramp 1500 in FIG. 12 (which is attached to wall 1391) to push against ramp 243 of stop 240. This may cause stop 240 to deflect about its fixed end 241 in the direction of arrow 240a, and allow shuttle 340 to move from its second position to its third position to retract needle 306 as set forth above. In this way, withdrawal and insertion of the needle into a patient can both be accomplished with a single spring within the device.

It is further contemplated that fluid conduit 300 may be the only fluid conduit of auto-injector 2 configured to be in fluid communication with cartridge 1302. Thus, drugs from cartridge 1302 may be deployed only through fluid conduit 300 and into the user during normal operation of auto-injector 2. Additionally, needle 306 may be the only needle of auto-injector 2 configured to be deployed into a patient. In this way, a single piece of metal or plastic can be used to carry the fluid from cartridge 1302 to a patient.

### Locking Component (Drop Pin)

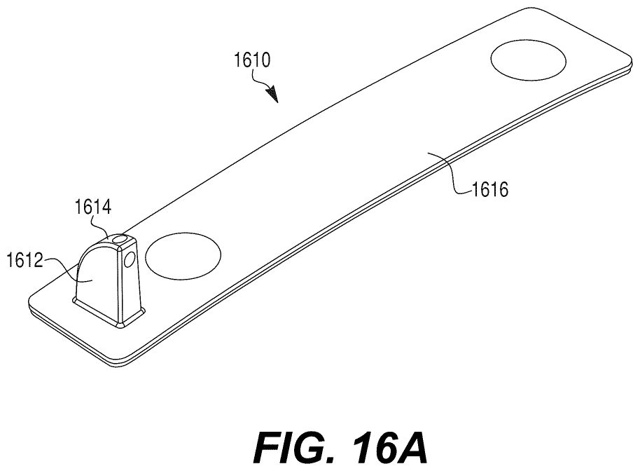

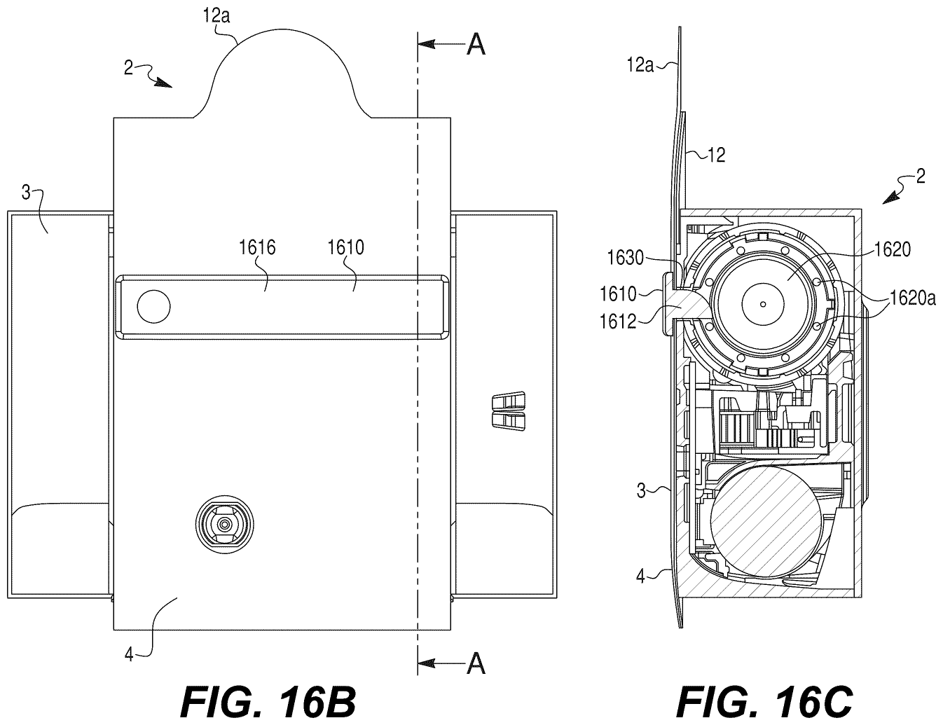

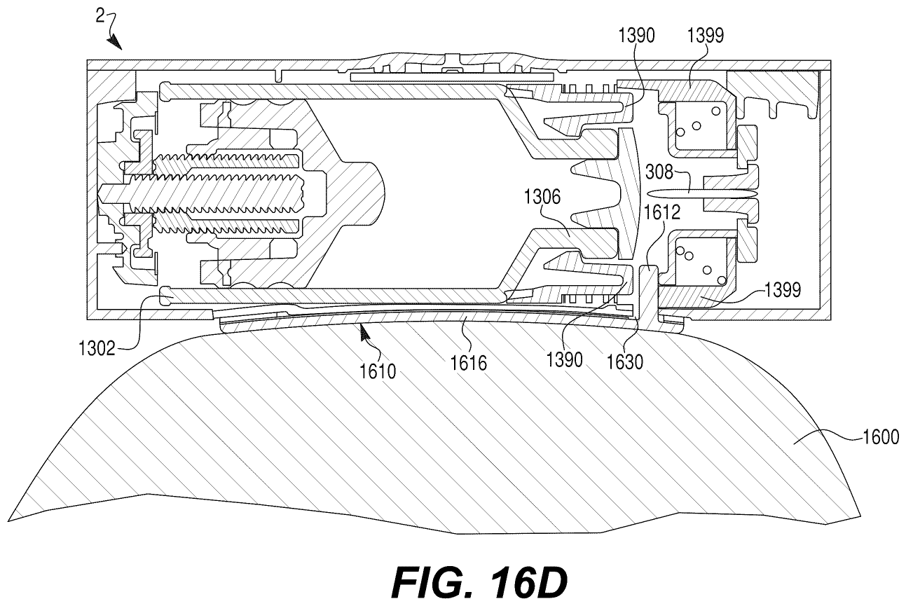

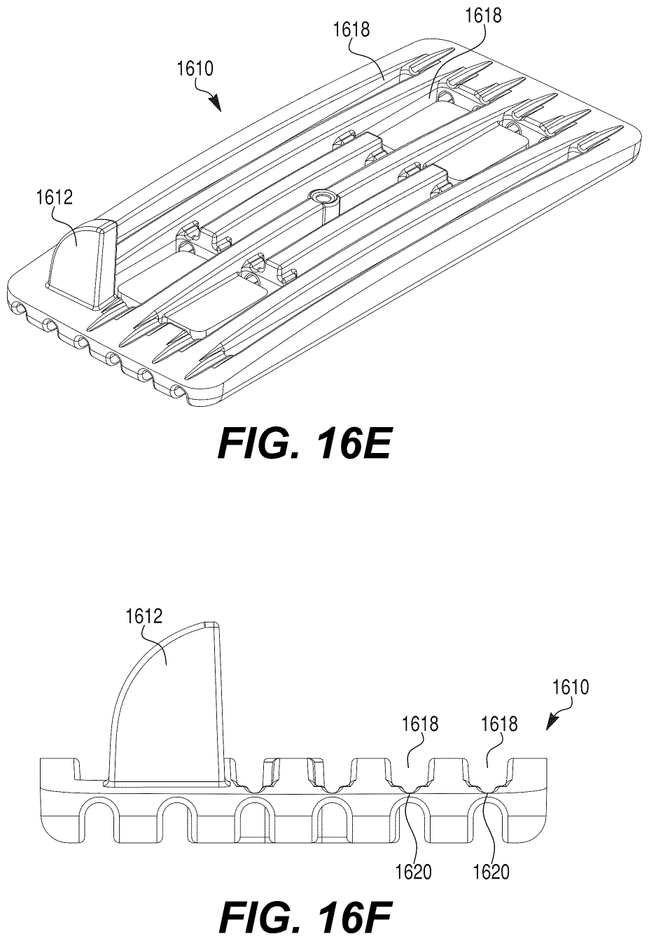

Referring to FIGS. 16A-16D, auto-injector 2 may include a locking component 1610. As shown in FIG. 16A, locking component 1610 may include a lock (e.g., a protrusion) 1612 having a curved surface 1614 and may further include a cover portion 1616. Cover portion 1616 may be shaped to conform to tissue-engaging surface 4 of auto-injector 2, as shown in FIG. 16D. Cover portion 1616 and tissue-engaging surface 4 may be concave to receive an anatomical portion 1600 of a user. Anatomical portion 1600 may be, for example, a thigh, hip, arm, posterior, or any other area of the body suitable for injection. Lock 1612 may be connected to cover portion 1616. In some embodiments, locking component 1610 may be formed as a single piece, such that lock 1612 and cover portion 1616 are integrally connected. Locking component 1610 may be formed from any suitable rigid or semi-rigid material. Locking component 1610 may, for example, be formed of methylmethacrylate acrylonitrile butadiene styrene (ABS) and may further have a frosted clear appearance indicating that locking component 1610 is disposable.

As shown in FIGS. 16B-16C, locking component 1610 may be disposed on or adjacent tissue-engaging surface 4 of the auto-injector 2 such that lock 1612 may extend into auto-injector 2. Locking component 1610 may further be disposed on or adjacent a liner 12a which may initially cover adhesive patch 12 prior to use of auto-injector 2. Locking component 1610 may be positioned relative to liner 12a such that upon removal of liner 12a from adhesive patch 12, locking component 1610 may also be removed from tissue-engaging surface 4. Lock 1612 may extend into auto-injector 2 via lock opening 1630 formed in tissue-engaging surface 4. When locking component 1610 is disposed on or adjacent tissue-engaging surface 4, cover portion 1616 may be attached to tissue-engaging surface 4 via an adhesive disposed between cover portion 1616 and tissue-engaging surface 4. Locking component 1610 may be disposed on or adjacent tissue-engaging surface 4 such that it is selectively removable by a user.

Referring to FIGS. 16C-D, when locking component 1610 is disposed on or adjacent tissue-engaging surface 4, lock 1612 may extend into auto-injector 2 such that it prevents movement of one or more internal mechanisms of auto-injector 2. For example, when locking component 1610 is disposed on or adjacent auto-injector 2, lock 1612 may extend into auto-injector 2 such that lock 1612 engages with one or more internal components of auto-injector 2, preventing those components from moving and/or being activated.

Referring to FIG. 16D, when locking component 1610 is disposed on or adjacent tissue-engaging surface 4, lock 1612 may extend into auto-injector 2 such that it is disposed within piercing system 1300. As described herein previously, collar 1390 may be coupled or fixed to second end 1306 of cartridge 1302. As also described herein previously, when piercing system 1300 is moved from the pre-activated state, cartridge 1302 and consequently collar 1390 may translate in a direction parallel to a longitudinal axis of cartridge 1302 toward retaining portion 1399. When lock 1612 extends into auto-injector 2 and is adjacent to collar 1390, lock 1612 may prevent cartridge 1302 from translating toward retaining portion 1399 or otherwise prevent cartridge 1302 and collar 1390 from applying a force against actuation portion 1394. Thus, even if the motor were somehow activated while lock 1612 is disposed in its locking position, fluid communication between needle 308 and cartridge 1302 could not be established and needle 306 could not be deployed outside of housing 3. Furthermore, when in the locking position, lock 1612 may prevent the movement of cartridge 1302 toward needle 308, thereby preventing deflection of actuation portion 1394 and consequently preventing retaining portion 1399 and needle 308 from moving toward cartridge 1302. In the event of auto-injector 2 being dropped or being subject to vibrations, lock 1612 may further prevent piercing system 1300 from being moved from the pre-activated state and consequently may prevent cartridge 1302 from being pierced by the needle 308.

When locking component 1610 is disposed on or adjacent tissue-engaging surface 4, locking component 1610 may additionally serve as a spacer between a user's skin and tissue-engaging surface 4. For example, locking component 1610 may have a thickness such that touch sensor 1410, described in greater detail hereinafter, is unable to detect the user's skin thereby avoiding inadvertent activation of auto-injector 2. Locking component 1610 may have a thickness, for example, from about 1 mm and about 5 mm, or about 3 mm.

Accordingly, locking component 1610 may act as an effective safety mechanism to prevent inadvertent activation of auto-injector 2. When locking component 1610 is disposed on or adjacent the tissue-engaging surface 4, lock 1612 may prevent various internal components of auto-injector 2 from moving. In the event auto-injector 2 is dropped on the floor prior to use, for example, locking component 1610 may prevent inadvertent piercing of cartridge 1302 and/or inadvertent initiation of an injection sequence. Locking component 1610 may also prevent such movement and/or inadvertent initiation of an injection sequence should auto-injector 2 be subjected to vibration during transport.

If a user wishes to use and/or is ready to use auto-injector 2, the user may separate locking component 1610 from tissue-engaging surface 4, thereby removing lock 1612 from lock opening 1630. The user may, for example, peel cover portion 1616 off of tissue-engaging surface 4. Alternatively, the user may peel liner 12a away from adhesive patch 12, thereby removing locking component 1610 from tissue-engaging surface 4. When separating locking component 1610 from tissue-engaging surface 4, curved surface 1614 may allow lock 1612 to rock within lock opening 1630, thereby allowing lock 1612 to be easily removed from lock opening 1630. With lock 1612 removed from lock opening 1630, auto-injector 2 may be in a state in which it is ready to be used such that, e.g., an injection sequence may be initiated.

FIGS. 16E and 16F depict locking component 1610 according to some embodiments. As shown in FIGS. 16E and 16F, locking component 1610 may have an increased width (relative to the depiction of locking component 1610 in FIGS. 16A-16C) to ensure locking component 1610 extends over touch sensor 1410 when locking component 1610 is disposed on auto-injector 2. Moreover, locking component 1610 may have a ribbed structure and may include air gaps or recesses 1618 and hinges 1620. Air gaps 1618 may inhibit conduction of a capacitive field between the user's skin and touch sensor 1410 when the auto-injector 2 is placed near the user with locking component 1610 in place. Hinges 1620 may allow locking component 1610 to flex when locking component 1610 is peeled from auto-injector 2. In some embodiments, a removable cover other than and/or separate from locking component 1610 may extend over touch sensor 1410 to inhibit inadvertent skin detection.

### Electronics

FIG. 4A shows a control system 1400 of auto-injector 2. Control system 1400 may include components positioned on a first electronics board 1402 and a second electronics board 1404, and also may include a power source 1406. First electronics board 1402 may include a controller 1408, an activating switch 1409, a touch sensor 1410, a needle insert switch 1412, and an emitter 1414. Second electronics board 1404 may include a detector 1416, an audio module 1418, a visual module 1420, and a haptic module 1422. Though FIG. 4A depicts audio module 1418, visual module 1420, and haptic module 1422 as included in second electronics board 1404, in some embodiments, one or more of the foregoing modules may be included on the first electronics board 1402 One or more of the components of first electronics board 1402 and second electronics board 1404 may be operatively coupled to controller 1408, and powered by power source 1406. Controller 1408 also may be operatively coupled to translation mechanism 1366, and may be configured to control operation of translation mechanism 1366 to initiate and control needle insertion and retraction as set forth above. Translation mechanism 1366 may be coupled to first electronics board 1402 via one or more spring contacts during a final assembly step where cartridge 1302 is inserted into housing 3. As described herein previously, translation mechanism 1366 may include a motor, gearing, and a leadscrew mechanism.

The majority of the assembly of auto-injector 2 may occur, e.g., on an assembly line at a manufacturing facility. Then, two device halves (or portions) may be shipped to a drug filling or final assembly facility. Indeed, the two separate portions 1490 and 1492 need not be the same size, as illustrated in FIG. 4B. Once a drug vial, e.g., cartridge 1302, is filled with a drug or other medicament, cartridge 1302 may be assembled with a remainder of auto-injector 2. For example, the two device halves (portions 1490 and 1492) may be assembled together with the filled drug cartridge 1302 therein. In one example, portion 1490 and translation mechanism 1366 may be snapped in place behind cartridge 1302. Portion 1490 may be part of housing 3 including a base or module configured to contain translation mechanism 1366 and its associated electronics. Portion 1492 may be a part of housing 3 containing substantially all of the other components described herein, including, e.g., the needle mechanism, sterile connector, and piercing mechanisms described herein. In this example, an electrical connection of the motor of translation mechanism 1366 must be made during the snapping of translation mechanism 1366 behind cartridge 1302 (i.e., during the assembly step where portions 1490 and 1492, and cartridge 1302 are combined to form a complete and functional auto-injector 2). To accommodate such an electrical connection, the drivetrain of translation mechanism 1366 may include one or more spring contacts 1494 (referring to FIG. 4C) that will contact pads 1495 (also referring to FIG. 4C) on the first electronics board 1402 upon assembly. Though not depicted in FIG. 4C, the drivetrain of translation mechanism 1366 may include additional spring contacts that may contact additional pads on the first electronic board 1402 upon assembly. Such additional spring contacts and additional pads may serve to connect additional components of the translation mechanism 1366, such as a tachometer, a motor encoder, or any other sensors or devices, to the first electronics board 1402. Thus, the connection of translation mechanism 1366 to first electronics board 1402 (including controller 1408) may be made without any loose wires or other similar structures.

Such an assembly process may be relatively simpler than simpler devices (e.g., auto-injectors) with relatively more complex final assembly processes. As a result, the contemplated assembly process described herein may lead to a reduction of labor costs.

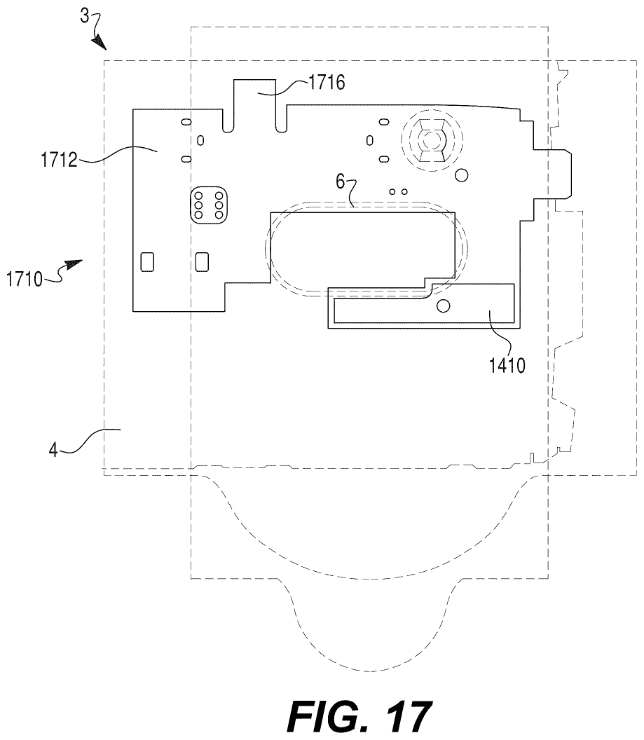

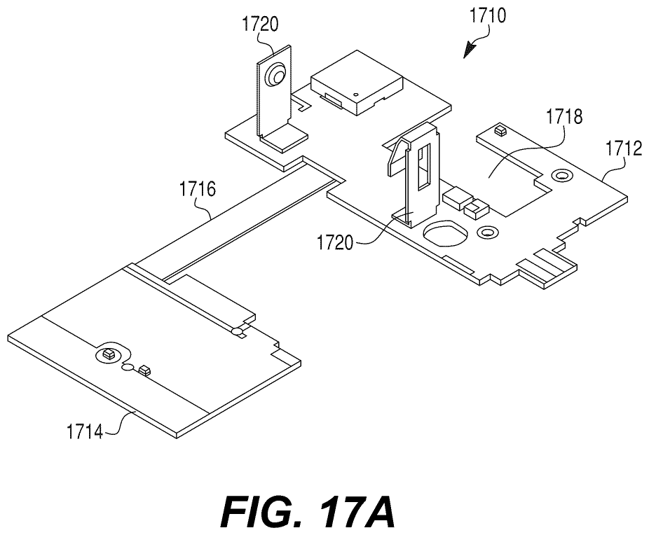

In some embodiments, auto-injector 2 may include a single (i.e., only or exactly one) electronics board 1710 as shown in FIGS. 17 and 17A, on which the components of control system 1400 described herein previously may be positioned. As shown in FIG. 17A, electronics board 1710 may include a first board segment 1712 and a second board segment 1714. The first board segment 1712 and the second board segment 1714 may be physically and electrically connected via a flexible segment 1716. Flexible segment 1716 may be, for example, a ribbon cable, a flexible conductive substrate, or the like. In some embodiments, flexible segment 1716 may be formed of fiberglass board that is machined thinly enough to flex, and is sometimes referred to as “semi-flex.” In some embodiments, flexible segment 1716 may be formed of a flexible polymer. The flexible polymer may be formed by a process sometimes referred to as “rigid-flex” in which a sandwich of a first portion fiberglass, flexible polymer, and second portion of fiberglass is first formed. The first and second portions of fiberglass may be subsequently removed to leave the thin flexible polymer portion.

Electronics board 1710 may include one or more brackets 1720 for mounting or otherwise securing electronics board 1710 to an interior of auto-injector 2. The first board segment 1712 may further include a cutout 1718. The cutout 1718 may be positioned such that first board segment 1712 may be positioned to allow the needle to pass through cutout 1718 when deployed.

In some embodiments, first board segment 1712 may correspond to first electronics board 1402 and second board segment 1714 may similarly correspond to second electronics board 1404, each as described herein previously. By connecting first board segment 1712 and second board segment 1714 via flexible segment 1716, first board segment 1712 may be positioned adjacent to tissue-engaging surface 4 of the auto-injector 2 whereas second board segment 1714 may be positioned on an opposite side of auto-injector 2 toward upper portion 30 of housing 3. Accordingly, the single electronics board 1710 may be utilized to both connect components located toward tissue-engaging surface 4 and connect components located toward upper portion 30. Such a configuration may allow for ease of assembly of the auto-injector 2 by obviating a need for complex wiring or soldering.

As shown in FIG. 17, electronics board 1710 may be positioned within housing 3. First board segment 1712 may be positioned adjacent to tissue-engaging surface 4 whereas second board segment 1714 may be positioned on an opposite side of auto-injector 2 (e.g. behind first board segment 1712 in FIG. 17). Flexible segment 1716 may be flexed or folded to maintain a connection between first board segment 1712 and second board segment 1714 in such positions.

Touch sensor 1410 may be incorporated in or on first board segment 1712 of electronics board 1710. To allow for adequate detection of a user's skin, touch sensor 1410 and first board segment 1712 may be located close to tissue-engaging surface 4 of housing 3. Tissue-engaging surface 4 of housing 3, or a portion thereof adjacent to touch sensor 1410, may be sufficiently thin such that an electric field of detectable magnitude may form between touch sensor 1410 and a user's skin. In some embodiments, the portion of tissue-engaging surface 4 adjacent touch sensor 1410 may be less than about 2 mm, about 1 mm, or less than about 1 mm. Further, the portion of tissue-engaging surface 4 adjacent touch sensor 1410 may be made from a solid material, such as plastic. By forming the portion of tissue-engaging surface 4 adjacent touch sensor 1410 from a solid material, as opposed to a ribbed, cored, or hollow material, a dielectric constant between the user's skin and touch sensor 1410 may optimize a responsiveness of touch sensor 1410.

Additionally, touch sensor 1410 may be positioned in or on electronics board 1710 so as to be adjacent to or near opening 6 through which the needle may be deployed. By positioning touch sensor 1410 adjacent to or near opening 6, a likelihood that touch sensor 1410 may detect a user's skin when auto-injector is positioned appropriately is increased. Furthermore, a curvature of tissue-engaging surface 4 may decrease the likelihood that touch sensor 1410 may falsely interpret a flat surface such as a tabletop to be a user's skin by creating a space between touch sensor 1410 and the flat surface.

By incorporating touch sensor 1410 in or on electronics board 1710, a need for one or more wires and/or other circuitry connecting touch sensor 1410 to a separate electronics board may be eliminated. Assembly of the auto-injector 2 may thereby be simplified and a cost of the auto-injector may be reduced.

As electronics board 1710 may be located adjacent to tissue-engaging surface 4, electronics board 1710 may include a cutout to allow the needle to be deployed through electronics board 1710 and subsequently through opening 6. Further, electronics board 1710 may be positioned such that touch sensor 1410 is directly adjacent opening 6 and no gap exists between an edge of touch sensor 1410 and opening 6. Alternatively, electronics board 1710 may be positioned such that a gap exists between an edge of touch sensor 1410 and opening 6 and the gap has a maximum of width of 5 mm, 2 mm, or 1 mm, for example.

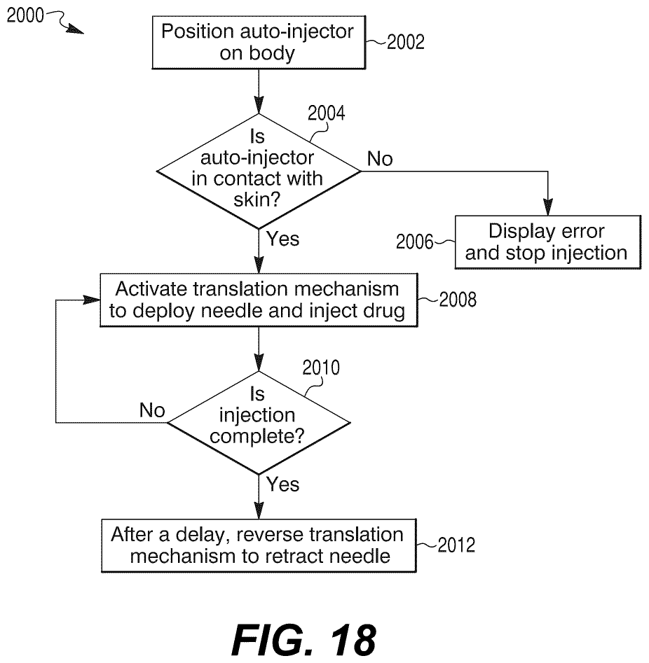

Controller 1408 may be configured to accept information from the system and system components described above, and process the information according to various algorithms to produce control signals for controlling internal mechanisms of auto-injector 2, including translation mechanism 1366. Examples of such algorithms are described hereinafter with reference to FIGS. 18 and 20-23. The processor may accept information from the system and system components, process the information according to various algorithms, and produce information signals that may be directed to audio module 1418, visual module 1420, haptic module 1422, or other indicators of, e.g., second electronics board 1404, in order to inform a user of the system status, component status, procedure status or any other useful information that is being monitored by the system. The processor may be a digital IC processor, analog processor or any other suitable logic or control system that carries out the control algorithms.

As discussed above with respect to FIGS. 3A and 3B, activating switch 1409 may be a mechanical plunger-type switch that extends away from tissue-engaging surface 4 of auto-injector 2. Activating switch 1409 may include an electrical circuit that is complete unless activating switch 1409 is depressed. For example, when auto-injector 2 is attached to a user's skin, switch 1409 may be depressed, breaking the electrical circuit, and indicating to controller 1408 that auto-injector 2 should be activated. In order to conserve power, the components of auto-injector 2 may be in an idle or sleep mode until switch 1409 is activated. In yet another example, auto-injector 2 may not be powered at all until switch 1409 is activated, and deactivation of switch 1409 may cut off power to auto-injector 2 entirely. While a mechanical plunger-type switch is disclosed, any other suitable mechanism for activating auto-injector 2 may be utilized, including, e.g., a button depressed by the user, voice signals, and a wireless signal from another electronic device, among others.

Touch sensor 1410 may be configured to help controller 1408 determine whether auto-injector 2 is properly deployed on the skin of a user. In one example, touch sensor 1410 may be a capacitive sensing electrode or any other device configured to differentiate contact with skin versus other materials, such as, e.g., wood, plastic, metal, or another material. When skin is in the proximity of the capacitive sensing electrode, a signal indicative of such contact may be sent to controller 1408. Thus, touch sensor 1410 may serve to verify that auto-injector 2 is properly placed on a user's skin, even if switch 1409 is depressed. Touch sensor 1410 may include a capacitive sensing electrode coupled to first electronics board 1402 and also to an interior of housing 3. Housing 3 and adhesive patch 12 may act as an overlay (insulator) that acts as a dielectric between the skin of the user and the capacitive sensing electrode. Alternatively, touch sensor 1410 may be incorporated in or on electronics board 1710, as described herein previously, such that the capacitive sensing electrode is also incorporated in or on electronics board 1710, Contact of portions of housing 3 and/or adhesive patch 12 near the capacitive sensing electrode may cause the capacitance of the electrode to increase, for example, by about 1 to about 10 pF, indicating placement of auto-injector 2 on a skin surface.

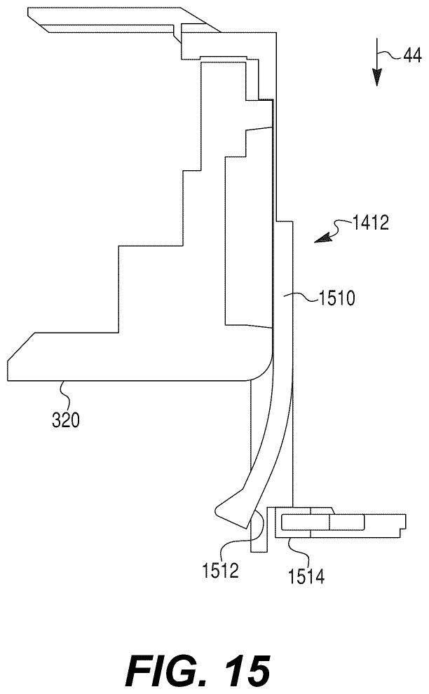

Needle insert switch 1412 may be configured to send a signal to controller 1408 that needle 306 is deployed within a user. For example, referring to FIG. 15, needle insert switch 1412 may include a curved cantilever 1510 including a first contact 1512. Needle insert switch 1412 also may include a second contact 1514. First contact 1512 may be placed into electrical contact with second contact 1514 when needle 306 is deployed into the user. During deployment of needle 306, driver 320 may move downward along axis 44 and deflect curved cantilever 1510 and first contact 1512 toward second contact 1514. When first contact 1512 and second contact 1514 connect to one another, a signal may be sent to controller 1408 indicating that needle 306 has been successfully deployed into the user. The separation of first contact 1512 and second contact 1514 may indicate that needle 306 has been retracted from the user.

Emitter 1414 and detector 1416 may operate as an optical interruption sensor, or photo-interrupter in order to allow controller 1408 to determine a state of auto-injector 2. Emitter 1414 may be a light emitting diode (LED) or other suitable light emitter, and detector 1416 may be, e.g., a phototransistor configured to receive light emitted by emitter 1414. In one example, emitter 1414 may emit infrared light, although other suitable wavelengths of light also may be used. The use of infrared light may help reduce interference from external light.

As shown in FIG. 13B, emitter 1414 and detector 1416 may be arranged across from one another within housing 3 to enable a beam of light 1430 to pass from emitter 1414, through cartridge 1302, to detector 1416. Cartridge 1302, and any fluid contained therein may be at least partially transparent to beam 1430 so that beam 1430 may pass through cartridge 1302 and its contents. As piston 1316 is moved toward second end 1306 during drug delivery (referring to FIGS. 13 and 14), piston 1316, and in particular a shoulder of piston 1316, may interrupt beam 1430. When detector 1416 fails to sense beam 1430, a signal may be sent to controller 1408, which may interpret the signal to indicate an end of an injection (e.g., that all of the drug contained within cartridge 1302 has been expelled). In some examples, the refraction path of beam 1430 may be considered when positioning emitter 1414 and detector 1416 relative to one another. For example, beam 1430 may be refracted as it passes through cartridge 1302 and any liquid contained therein, and emitter 1414 and detector 1416 may be offset from one another accordingly. Additionally, emitter 1414 and detector 1416 may be offset from a center of housing 3 so that the shoulder of piston 1316 may block beam 1430. In at least some examples, an optical interruption sensor or similar mechanism may help avoid false positives in the event of a drive train failure. That is, the optical switch may help controller 1408 determine that an injection was not completed with greater accuracy than other mechanisms.

Audio module 1418 may include a speaker or the like to provide audio feedback to the user. Openings in housing 3 may facilitate the travel of sound from audio module 1418 to the user. Audio module 1418 may generate a tone or other sound at the start and at the end of injection, and/or to indicate any other benchmark during the injection, such as an error, for example. Visual module 1420 may include one or more LEDs or similar devices to provide visual feedback to the user. Visual module 1420 may include different colored LEDs to provide various messages to the user. For example, a plurality of blue LEDs arranged in a ring could be used to display progress of the injection over time, one or more green LEDs could be used to display completion of the injection, and a red LED could be used to display an error to the user. Any other suitable colors, combinations, and/or numbers of LEDs may be used in various examples. For example, a combination of red, blue, and purple LEDs may be utilized. In one arrangement, eight LEDs may be arranged in a circle having a diameter of about 26.5 mm, or a diameter from about 10.0 mm to about 40.0 mm. It is to be understood that this exemplary quantity and positioning of LEDs is not intended to be limiting and any quantity and/or positioning of LEDs may be used. The LEDs may be activated sequentially around the circle to indicate progress of an injection (e.g., in a progress ring arranged in a similar manner as a clock-see, for example, LEDs 52 on FIG. 4B). Controller 1408 also may be configured to receive feedback from various sensors, and rescale a speed that various LEDs are activated based on feedback from the sensors. For example, the LEDs in the progress ring may be activated in three or more operation phases including, e.g., an injection sequence activation phase, an injection phase, and a retraction phase. Those of ordinary skill in the art will recognize that auto-injector 2 may have more or less than the above-described three operation phases. There may be an expected time for completing each phase, but there also may be some variability in the actual times experienced during any of the aforementioned operation phases of auto-injector 2. An algorithm may be utilized to help avoid the premature activation of LEDs, for example, when a certain phase finishes earlier than expected, or to have progress along the ring stopped when a certain phase takes longer than expected. At any given point, the algorithm may divide the remaining estimated time for completion of drug delivery by the number of unactivated LEDs in the progress ring, to determine a rate at which the remaining LEDs in the progress ring should be activated.

For example, before the injection sequence activation phase, the LEDs may be activated at a rate equal to the estimated time of the entire drug delivery process (e.g., the estimated time to complete all of injection sequence activation phase, the injection phase, and the retraction phase) divided by the total number of unactivated LEDs in the progress ring. Stated differently, the estimated time of the entire drug delivery process may be divided by a number that is the total number of LEDs in the progress ring less any already-activated LEDs. Thus, if, for example, one LED is already activated, the estimated time of the entire drug delivery process may be divided by one less than the total number of LEDs in the progress ring.

After completion of the injection sequence activation phase, the LEDs may be activated at a rate equal to the sum of estimated times for completing the remaining phases (e.g., the injection phase and the retraction phase) divided by the number of unlit LEDs in the progress ring. After completion of the injection phase, the LEDs may be activated at a rate equal to the estimated time to complete the retraction phase, divided by the number of unlit LEDs.

In some embodiments, subsets of LEDs may be used to indicate progress of injection phases. For example, in embodiments having eight LEDs positioned on a housing of auto-injector 2, a first LED may be illuminated to indicate needle insertion. The second through seventh LEDs may then be illuminated sequentially to indicate a progress of the injection phase. Lastly, the eighth LED may be illuminated to indicate needle retraction. While an exemplary configuration of the LEDs and corresponding logic has been described, it should be understood that the quantities of LEDs for each phase of an injection process may be varied as desired.

Visual module 1420 also may include a display screen, touch screen, or other suitable device to provide one-way or two-way communication with the user. Visual module 1420 may be visible by the user from outside of housing 3 via a window in housing 3. Haptic module 1422 may include, e.g., a haptic motor configured to generate vibrations that can be felt by the user. Vibrations may signal the start and the end of an injection, and/or may help provide additional information to a user.

Controller 1408 may be coupled to a wireless communication module and an antenna. The wireless communication module may be configured to transmit data from controller 1408 to, e.g., the mobile device 1407, computer, cell phone, or the like. The mobile device 1407 may include any appropriate mobile device, e.g., smart phones, laptops, smart watches, head sets, etc. The mobile device 1407 may allow a user 1449 to text, call, internet search etc. The wireless communication module may be configured to transmit information over one or more wireless modalities, such as, e.g., Bluetooth, Bluetooth low energy (BLE), near-field communication (NFC), infrared, cellular networks, and wireless networks, among others. The antenna may be any suitable device configured to assist the wireless communication module in data transmission and/or amplification. Thus, controller 1408 may be configured to transmit diagnostic information of the user and/or auto-injector 2, information pertaining to completion of an injection, and/or information pertaining to an error state of auto-injector 2 to a device of the user, or to the cloud. Signals indicative of needle insertion and/or early device removal also could be transmitted via the wireless communication module. Controller 1408 may also be configured to transmit temperature information for auto-injector 2. For example, a user may be able to monitor, via a mobile device 1407 and/or application, for example, a temperature of auto-injector 2 when auto-injector 2 is removed from refrigeration. A user may also be able to monitor, via a mobile device 1407 and/or application, a temperature of the medicament within auto-injector 2. The mobile device 1407 and/or application may be configured to indicate to the user whether the medicament has reached a temperature suitable for administration. Additionally, the mobile device 1407 and/or application may be configured to indicate to the user whether the medicament has reached a temperature, such as an unsuitably high temperature, that may compromise the medicament. Controller 1408 also may receive activation and/or delay commands via the wireless communication module. Controller 1408 may further receive operation adjustment commands such as commands relating to adjustment of preferred operation speed, for example. In some embodiments, controller 1408 may receive a command to increase the speed of injection, or decrease the speed of injection, or pause an injection.

In some embodiments, controller 1408 may communicate with a mobile application of a user's mobile device via the wireless communication module. The mobile application may be configured to facilitate use of auto-injector 2 and improve user experience. In some embodiments, the mobile application may be used to automatically check an expiration date of a medicament contained within auto-injector 2. Such functionality may relieve a user from having to manually check the expiration date and may improve user safety. Based on an expiration date, the mobile device may be configured to alert the user and/or disable use of the auto-injector 2. In some embodiments, the mobile application may be used to alert a user as to product recalls and/or may disable the device in the event of product recalls. For example, the mobile application may access a database via the internet to determine whether particular devices, lots of devices, medicaments, and/or lots of medicaments have been recalled. In some embodiments, the mobile application may be configured to confirm whether the auto-injector 2 and/or medicament is authentic as opposed to counterfeit. The mobile application may do so by, for example, cross-referencing a product serial number or a digital signature against a database of authenticated products. In some embodiments, a portion or portions of auto-injector 2 may be disposable and the mobile application may be configured to confirm the authenticity of such portion or portions prior to use.

In some embodiments, the mobile application may be used to facilitate an injection sequence. For example, the mobile application may sync with the events of an injection sequence and provide contemporaneous instructions to the user as to which tasks (e.g., depress switch 1409, hold auto-injector 2 against skin, remove auto-injector 2) to perform at which times. The mobile application may provide injection training to users 1449. In some embodiments, the instructions may be narrated audibly. In some embodiments, the instructions may be provided visually via a display on the mobile device. In some embodiments, the mobile application may be configured to provide a detailed indication of a progress of an injection sequence. For example, the mobile application may provide text, visual, and/or audible indications of progress with greater granularity than shown by LEDs, for example, as described herein previously.

In some embodiments, the mobile application may enable the user to control functionality of auto-injector 2 via the provision of commands. More particularly, a user may provide commands (e.g., provided by one or more input means including touch input, keyboard input, stylus input, voice input, any combination thereof, etc.) to the mobile application, which are subsequently transmitted to auto-injector 2. For example, the user may control when the injection sequence starts by providing an initiation command. As another example, the user may pause an active injection sequence by providing a pause command to the mobile application. In yet another example, the user may issue a command to stop/abort the injection sequence. In some embodiments, the mobile application may be used to control customizable operating parameters of auto-injector 2. For example, the user may adjust a speed of needle insertion and/or a speed of medicament dispensation via the mobile application. In some embodiments, the user may adjust a depth of needle insertion via the mobile application. In some embodiments, the user may enable, disable, and/or personalize visual and audible feedback from auto-injector 2 via the mobile application.

In some embodiments, the mobile application may be configured to record and store a date and/or time of an injection. Based on the date and/or time of the injection, and a user's prescription information, the mobile application may be configured to automatically create a reminder for a subsequent injection. In some embodiments, the mobile application may be configured to receive (e.g., at predetermined intervals, in response to predetermined events, etc.) various types of context data associated with the user (e.g., schedule information, habit information, etc.). The mobile application may leverage this context data to provide reminders at the most optimal times (e.g., when the context data indicates that the user is likely at home instead of at the gym, etc.). In some embodiments, upon completion of an injection, the mobile application may be configured to provide a notification to the user with positive feedback for adherence to a prescription regimen. In some embodiments, the mobile application may provide points and/or rewards for continued adherence.

In some embodiments, the mobile application may be configured to authenticate a user of the auto-injector 2 prior to use. For example, the mobile application, in connection with the user's mobile device, may use biometric identification, two-factor authentication, or any other suitable authentication protocol to confirm the identity of the user prior to an injection. Upon authentication of the user, the mobile application may cause the auto-injector to become activated or otherwise be unlocked. Such user authentication may inhibit misuse and/or waste of costly medicaments by persons other than an intended user.

In some embodiments, the mobile application may be configured to detect operating conditions of auto-injector 2. For example, the mobile application may be configured to detect a battery level of the device and in case of a low battery indication, the mobile application may be configured to provide a notification to the user indicative of a need to charge the device. In some embodiments, the mobile application may be configured to detect mechanical and/or electrical malfunctions of auto-injector 2 and convey such information to the user. In some embodiments, the mobile application may be apprised by auto-injector 2 whether a part or cartridge was inserted incorrectly. The mobile application may then be configured to provide a notification to the user of the incorrect assembly and/or may provide instructions to user that detail how to properly insert, connect, or position various components of auto-injector 2.

In an aspect, auto-injector 2 may be configured to track data associated with various types of errors that may be encountered during usage. Non-limiting examples of error-associated data include: the designation of the type of error identified by auto-injector 2 (e.g., hardware error, software error, user procedural error, etc.), when that error occurred during the treatment administration process (e.g., before, after, during, etc.), the amount of medicament delivered despite the error (e.g., no medicament, some medicament, including, for example, precise or estimated volumes, all medicament, etc.), whether the error is fixable (e.g., by a user, by a technician, etc.), and the like. In one aspect, this error data may be stored locally on the device and later retrieved. In another aspect, the error data may be transmitted (e.g., via a wired or wireless connection) to one or more other devices and/or applications (e.g., in substantially real-time as the error occurs). For example, some or all of the error data may be transmitted to a HCP, which may thereafter log the error data and/or take subsequent steps to remedy the error (e.g., transmit a notification to a patient device that instructs the patient how to fix the error, deploy a technician or other individual to the patient to remedy the error or replace the device).

Data associated with dose completion and status may also be tracked. Non-limiting examples of dose-related data include: the rate of dose delivery, the total time needed for the dose to be fully administered, the total time remaining for a current dose to be delivered, the date the dose was delivered, the location where the dose was delivered, and/or the total volume of the dose that was delivered. Multiple dose administration events may be compared against one another to identify similarities and/or differences between events. These comparisons may provide additional context information related to device operation and/or user schedule adherence. For example, if the dosage data indicates that the rate of dose delivery varies from one dose administration event to the next, an implication may be gleaned that there is an issue with auto-injector 2 and/or components therein. As another example, dosage data may indicate that a patient consistently takes their dose while in a supervised setting (e.g., at a clinic, at a hospital, etc.) but does not consistently take their dose while at home. In such a situation, the patient may be monitored more closely at home to ensure they are adhering to the prescribed dosing schedule to ensure optimal results.

Dose preparation data may also be tracked to ensure accurate dose delivery for each individual patient. For example, general information about each patient's auto-injector may be obtained by, for example, scanning a QR code associated with the patient's auto-injector (e.g., a QR code embodied on a sticker that is placed on an exterior surface of the auto-injector). Scanning of the QR code (e.g., by a mobile device, etc.) may reveal various types of information such as the auto-injector serial number, component IDs, expiration date of any components and/or medicaments contained therein, details of the patient that the auto-injector is assigned to, dose frequency per prescription, and the like. Auto-injector 2 may also be configured to track when the last dose was administered and may automatically transmit an indication to the mobile application and/or a designated device to issue a refill. Auto-injector 2 may also be configured to track device usage data (e.g., drug warm up time, drug temperature at different points before, after, or doing administration, dose administration frequency, etc.) and provide that information to the user and/or the HCP via the mobile application and/or one or more audio and/or visual prompts integrated with auto-injector 2. If auto-injector 2 detects issues and/or errors in usage (e.g. if patient dose changes or wrong prescription, a patient selects the incorrect auto-injector to use if there are multiple auto-injectors in the household, etc.) then auto-injector 2 may be configured to dynamically deactivate to prevent incorrect use. Additionally or alternatively, auto-injector 2 may transmit an indication to the mobile application that an error or issue has occurred that requires attention, which may later be addressed by the patient or the HCP.

To further ensure that the correct auto-injector is being utilized by the appropriate patient, certain safeguards may be instituted. For example, a QR code (e.g., present on a bracelet issued to the patient, etc.) may initially be scanned (e.g., by a device administrator) to assign a device to a patient. The patient may subsequently be required to have the QR code scanned (e.g., by the auto-injector, by another device connected to the auto-injector, etc.) to ensure they are authorized to receive the treatment. If authorized, the patient may thereafter utilize auto-injector 2 to receive the treatment. If not authorized, the patient may be prevented from receiving an injection administration from auto-injector 2. In an aspect, a time component to the scanning may be present such that a patient may be eligible to receive treatment only at predetermined times. More particularly, if a patient scans the QR code outside of their prescribed time interval, or scans the QR code more than a predetermined number of times within a specific time window (e.g., more than once per day, etc.), then they may be precluded from receiving treatment.

Auto-injector 2 may also be configured to track the locations on the patient's body where previous injections occurred and may subsequently provide (e.g., via the mobile application) recommendations on new injection sites (e.g., based on the location of last injection and/or disease state, etc.). For example, if the patient previously received one or more injections on their left arm, auto-injector 2 may transmit a recommendation (e.g., to a patient's device via a mobile application) to the patient to administer the next injection to a location where they have not previously received injections or have not received injections for a predetermined period of time (e.g., on their right arm). As another example, if the patient is afflicted with a particular disease state, auto-injector 2 may provide an injection site recommendation to deliver the medicament to a particular area of the body that may be most optimal for treating the disease. In yet another example, the mobile application may provide a signal to the user to identify a tissue injection site having firmer tissue based on the perceived/sensed depression of the plunger. For instance, if the mobile application receives an indication from auto-injector 2 that the plunger is not being depressed fully or consistently over a predetermined time span, then the mobile application may correlate this indication with a determination that the tissue at the injection site is not optimal for injection and may subsequently transmit a notification to the user informing them of this determination and/or providing a recommendation for an alternate injection site (e.g., one with firmer tissue).

Certain sales-related data associated with auto-injector 2 may also be tracked. For instance, the number of auto-injectors in stock at particular locations (e.g., clinic, hospital, other type of retailer, etc.) may continuously be tracked to ensure that locations do not run out of stock. In one aspect, when the number of auto-injectors at a particular location is getting low (e.g., the number available has fallen below a predetermined threshold, etc.) then the system may be configured to automatically place an order for more auto-injectors from a supplier.

Auto-injector 2 may also be configured to track various types of data associated with medicament delivery. For example, auto-injector 2 may be configured to track: how deep an injection needle 306 was inserted into the skin (e.g. subcutaneous layer, intramuscular layer, etc.), a strength of the adhesive that secures auto-injector 2 to the patient's body (e.g., by monitoring a pressure profile, receiving body type indications, receiving success indicators per injection site, etc.), an orientation of auto-injector 2 during the injection process (e.g., as tracked by an integrated accelerometer, etc.), and the like. Artificial intelligence (AI) software may also be leveraged to assess whether HCP support is needed based on the characteristics of the injection delivery. For example, Al may detect that a particular injection is going too deep into the patient (e.g., from data obtained from a gyroscope integrated into auto-injector 2, another type of depth measurement mechanism, etc.), and/or that the auto-injector is not positioned correctly relative to the patient's body, and may dynamically provide an alert on the mobile application accessible by the patient and/or to another device associated with a responsible attendant.

From a HCPs perspective, the collective information obtained from multiple auto-injectors that are being utilized by a patient group may enable the HCP to track the status of all treatments of multiple patients simultaneously. For example, an HCP may monitor the status of the injection process for each patient. If there is an issue with the injection process for any of the patients, the HCP may be apprised of the location of the patient (e.g., if they leave the building) to more quickly attend to the patient. Patient location data may be obtained, e.g., via the device QR code and/or by tracking a patient hospital band or medical ID bracelet. Additionally or alternatively, in certain aspects, the HCP may compare the collective data to identify which patients, or groups of patients, are developing adverse reactions to the treatments, are faring better than others, and the like.

In some instances, the mobile application may prompt the patient for additional context data that may subsequently be utilized to improve auto-injector 2 and/or the patient experience. For example, the patient may be prompted to: report on their usage experience (e.g., pain level, leakages observed, comfort level, bolus size, who applied the device (e.g., caretaker), the site the device was applied to (e.g., thigh, abdomen), capture a picture of the administration site (e.g., for HCP or clinician assessment later), provide an indication of their medication list (e.g., to monitor for interaction occurrences), provide biometric information (e.g., blood pressure, heart rate, etc.) for adverse event analysis, and/or provide disease progression information based on their treatment. In an aspect, patients may be prompted to provide their treatment experience inputs: each time they receive treatment, after an adverse reaction to treatment is identified, after a usage error has been detected, etc.

Other types of data may be tracked by auto-injector 2 and/or transmitted to the mobile application and/or one or more other designated devices. For example, geographic data for all distributed auto-injectors may be tracked to provide indications of the locations where there is a greater apparent need for auto-injectors to inform manufacturing and supply chain decisions. Purchase information for auto-injectors may be recorded to ensure there is no shorting supplies in any locations or populations. Additionally or alternatively, in some aspects, the geographic data may be leverage to inform other treatment decisions. For example, if some or many patients associated with a particular geographic region are experiencing adverse reactions to a particular form of treatment, then the HCP may be prompted to investigate whether characteristics of the treatment provided to those patients may need to be adjusted (e.g., in consideration of various environmental factors, etc.).

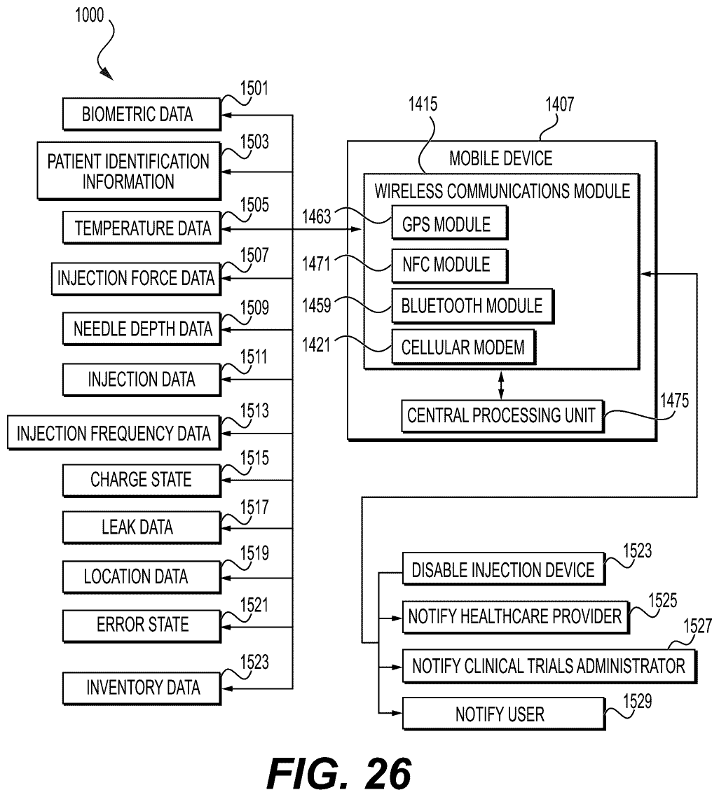

FIGS. 26-50 collectively provide details of non-limiting data types that may be tracked by auto-injector 2 and/or transmitted to one or more other devices and/or applications for presentation. For example, various types of visuals (e.g., in the form of charts and graphs) may be generated for any of the tracked data (e.g., graphs showing dates of device usage, volumes of medicament delivered per use, etc.). These charts may enable a viewer to more quickly understand how consistently a medicament is taken by a patient, how accurately the medicament is delivered (e.g., is substantially the same volume of the medicament delivered each time, does the delivered volume vary per administration, etc.), and the like. Data may also be tracked that catalogs a patient's hesitations and/or usage concerns. These comments may be relied on to subsequently adjust aspects of auto-injector 2 to improve device functionality and the user usage experience.

Auto-injector 2 may be configured to detect patient reaction symptoms at the injection site. For example, heat and/or swelling of the injection site may be detected, which may be an indication that an adverse reaction is occurring if the heat and/or swelling is greater than a predetermined threshold. For instance, a temperature sensor may be positioned at a skin contacting surface proximate to the injection site that is configured to measure changes in temperature from the initial point that needle 306 contacts the skin throughout the injection process. Changes in skin temperature above a predetermined threshold temperature, or changes in skin temperature occurring within a predetermined period of time, may be indicative of a patient reaction. In an aspect, the location (e.g., at home, in the hospital, etc.) where the patient is receiving the injection may be logged. Characteristics associated with injections administered at multiple location points may be compared to determine whether one location is “better” than another with respect to correct device operation, better patient experience, etc.

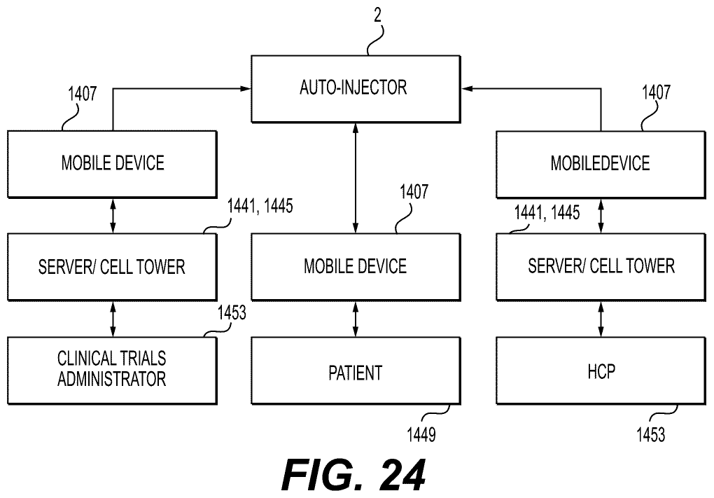

FIG. 24 discloses various ways to collect and store data associated with auto-injector 2 from three perspectives: a patient end perspective, an HCP perspective, and a clinician/clinical study perspective.

With respect to the patient end, data may be collected and transmitted using one or more sensors, components, and/or protocols associated with the device itself (e.g., a camera, global positioning system (GPS) 1463, accelerometer 1469, Bluetooth beacon, printer, near-field communication (NFC) antenna 1471, a gyroscope 1467, wired communications (e.g., a USB port 1473) and the like. Additionally or alternatively, one or more external devices may be configured to communicate with the electronics integrated within auto-injector 2 (e.g., various mobile devices such as phones, tablets, laptops and/or hybrid devices, USB, microUSB, etc.) to receive and/or capture the aforementioned data types. Possible communication modalities between the external devices and auto-injector 2 may be facilitated by Bluetooth, NFC, a physical connection, and the like. Users may interact with applications resident on these devices to enter and receive information, update calendar data, scan QR codes, capture images of relevant administration events (e.g., injection-site images, etc.), and the like. In some aspects, auto-injector 2 may be compatible with certain add-on device. For example, a polaroid device may be configured to connect to auto-injector 2 to print out various types of images and/or data. As another example, a medical/hospital bracelet may wirelessly connect to auto-injector 2 and may also include a camera component to scan QR code on bracelet. In some aspects, bracelet may include a micro-USB to be scanned that can communicate with auto-injector 2 to store data. For elderly patients, additional connectivity features are contemplated that may improve the user experience. For instance, having knowledge that a patient is above a predetermined age and/or has certain limited capabilities, auto-injector 2 may be configured to send a signal to an HCP service to trigger calls to patient's landline (e.g., if the patient does not have a mobile device, etc.). In a similar vein, auto-injector 2 may be configured to send information to the HCP for the patient's medical records and/or automatically update patient records and mail a letter summary to the patient.

With respect to the HCP perspective, various tools may be leveraged to acquire, store, and/or display the auto-injector data. For example, substantially real-time data associated with one or more patients may be tracked and presented on various devices (e.g., monitors, laptops, pagers, TVs, etc.) to ensure patient compliance (e.g., with an injection schedule). Data obtained by auto-injector 2 may be stored on the device itself (e.g., within an integrated database, etc.) and manually retrieved at a later time, may be provided to an add-on device or external component, and/or may be transmitted wirelessly to another storage location (e.g., a server, etc.). The frequency at which data may be transmitted to another device may vary (e.g., data may be transmitted each time it is collected, only once a predetermined amount of data has been collected, upon detection of a predetermined event (e.g., a command receipt from an HCP or other user to transmit the data), and the like).

With respect to the clinical study perspective, data transmission to an external database (e.g., via Bluetooth) may enable live updates to be provided on trends, errors, correlations between data types, and the like. If the data is physically stored on the device itself then it may be manually extracted and uploaded to a central data processor for analysis.

FIGS. 26-50 collectively provide details of non-limiting ways to collect and store auto-injector data from the patient, HCP, and/or clinician/clinical study perspective.

From the patient perspective, in an aspect, some or all of the acquired data on the patient device may be compiled into a PDF report that may be sent via email, text, etc. In another aspect, once an injection is complete, a notification may be transmitted to the patient's device (or may be present on the mobile application that is accessible via the patient's device) that may prompt the patient to fill out a log or questionnaire to track their patient experience. In another aspect, a patient's calendar may be automatically updated to reflect the next dose administration event. In another aspect, a notification may be transmitted to the patient reminding them of an upcoming dose administration event.

From the HCP perspective, in an aspect, a patient's file may be auto-populated with data associated with each dose. This data may be propagated to each individual (e.g., nurse, etc.) that may have patient contact and/or may be responsible for patient upkeep. The patient's may have some control over which types of data are automatically synced to an HCP portal. In another aspect, the HCP may receive an alert if the patient needed help administering a dose. This information may be utilized by the HCP to improve subsequent patient experiences (e.g., by scheduling to have a nurse or other individual present at the designated dose administration time).

From the clinician perspective, in an aspect, a clinical study portal may exist that may be configured to auto-populate data in to a patient-specific database. The clinical study portal may keep track of any alerts that occurred during the usage experience and/or any adverse reactions that may have developed in the patient during or after use. In another aspect, the clinical study portal may identify any trends occurring in the patient data and these trends may be automatically updated upon receipt of new data.

In some embodiments, auto-injector 2 may be configured to assist a user in locating auto-injector 2. For example, auto-injector 2 may be configured to pair with a mobile device before use. Auto-injector 2 may be configured such that, while in packaging before use, auto-injector 2 is in a low-power mode in which wireless connectivity is enabled. As described herein previously, auto-injector 2 may be enabled with any one or more suitable wireless modalities, such as, e.g., a Bluetooth or Bluetooth low energy (BLE) module 1457, a near-field communication (NFC) module 1417, infrared, cellular network connectivity (e.g., a cellular modem 1421), and wireless network connectivity (e.g., a WiFi module or network card 1419). In some embodiments, auto-injector 2 or its packaging may be provided to a user with a QR code or bar code. The user may scan the QR code or bar code with a mobile device to pair the mobile device with auto-injector 2. Alternatively, or in addition, auto-injector 2 may be in pairing mode by default when in the packaging in low-power mode. In such a case, the user may detect auto-injector 2 with a mobile device when the mobile device is brought into proximity with auto-injector 2. The user may then be prompted to pair auto-injector 2 with the mobile device. In still other embodiments, auto-injector 2 may be pre-configured to pair with a particular user's mobile device. For example, a HCP may pre-configure auto-injector 2 to be associated with a user account of a mobile application. The user may use the mobile application to detect and pair with auto-injector 2 via a unique and/or encrypted communications channel.

Once the user's mobile device and auto-injector 2 are paired, the user may use the mobile device to find auto-injector 2 in case it is misplaced. In some embodiments, the user may cause, via the mobile device, lights on auto-injector 2 to become illuminated and/or may cause auto-injector 2 to emit sounds. Additionally, the user may cause, via the mobile device, auto-injector 2 to emit haptic feedback, such as vibrations. The user may then use the lights, sounds, and/or haptic feedback to locate the device. In some embodiments, the user may track a geographic location of auto-injector 2 using the mobile device. The mobile device may depict a position of auto-injector 2 on a map using wireless connectivity with auto-injector 2 and/or GPS capability.

In some embodiments, auto-injector 2 may be configured to alert the user in connection with an attempt to locate auto-injector 2 as to whether auto-injector 2 and/or a medicament therein have been compromised. For example, auto-injector 2 may transmit information to the mobile device indicating that auto-injector 2 has been exposed to unsuitable environment, such as an excessively high temperature or excessively low temperature environment. In some embodiments, auto-injector 2 may transmit information to the mobile device indicating a rate of temperature change of the medicament within auto-injector 2. In such embodiments, a warning may be provided to the user via the mobile device indicating that a temperature of the medicament is approaching a threshold beyond which the medicament is unsafe to administer. The warning may include an estimated time to reach the threshold.

In some embodiments, auto-injector 2 may transmit information to the mobile device 1407 indicating that auto-injector 2 and/or the associated packaging has been tampered with during a pre-determined period of time prior to activating the location functionality. In some examples, auto-injector 2 may transmit information to the mobile device 1407 indicating that auto-injector 2 has been moved in an unsuitable manner. For example, auto-injector 2 may transmit information indicating that it has been dropped or otherwise sustained an impact that may compromise its functionality. In some embodiments, if auto-injector 2 has been used to complete an injection sequence, auto-injector 2 may transmit an indication to the mobile device that it has been used. In some embodiments, device location functionality may be disabled following an injection sequence.

In some embodiments, auto-injector 2 may be configured to provide location information to parties other than the user, such as a manufacturer of the device, a HCP, a pharmacy, or the like. For example, location functionality may be enabled prior to shipment of auto-injector 2. Auto-injector 2 may be configured, prior to shipment, to provide location information to the manufacturer, a HCP, a pharmacy, or the like. After shipment, the relevant party may track the location of auto-injector 2 to ensure that it is routed as expected. The relevant party may track a location of auto-injector 2 to inhibit theft and/or fraud, for example. In some embodiments, the relevant party may be automatically warned if auto-injector 2 is diverted from an expected or predetermined route.

FIG. 18 shows an exemplary method 2000 according to the disclosure. Method 2000 may start at step 2002, where a user may position auto-injector 2 on her body so that tissue-engaging surface 4 contacts a skin surface. The user may position auto-injector 2 on her skin after removing locking component 1610, as described herein previously, thereby allowing touch sensor 1410 to detect proximity of the skin. Auto-injector 2 may be mounted in any suitable location, such as, e.g., the thigh, abdomen, shoulder, forearm, upper arm, leg, buttocks, or another suitable location. Auto-injector 2 may be secured to the skin by adhesive patch 12. The securement of auto-injector 2 at step 2002 may cause activating switch 1409, which extends outward from tissue-engaging surface 4, to be depressed and break a circuit. The breaking of the circuit may cause a signal to be sent to controller 1408 indicative that activating switch 1409 has been depressed. Alternatively, any other suitable mechanism may power on or otherwise activate auto-injector 2 before or after step 2002. Upon depression of activating switch 1409, auto-injector 2 may emit an audio tone and/or illuminate one or more LEDs (e.g., one or more LEDs of a first color, e.g., blue) to indicate depression of activating switch 1409.

Once auto-injector 2 is activated at step 2002, method 2000 may proceed to step 2004, where controller 1408 may determine whether tissue-engaging surface 4 is positioned on a skin surface. At step 2004, controller 1408 may receive a measurement from touch sensor 1410 indicating whether auto-injector 2 is positioned on skin or another surface. If controller 1408 determines that touch sensor 1410 is in contact with skin, for example, when a capacitance value received from touch sensor 1410 is within a predetermined range, method 2000 may proceed to step 2008. If controller 1408 determines that touch sensor is not in contact with skin, for example, if the capacitance measurement received from touch sensor 1410 indicates that auto-injector 2 is in contact with a non-skin surface like wood or metal, method 2000 may proceed to step 2006. At step 2006, auto-injector 2 may be placed into an error condition. In the error condition, an LED may be activated (e.g., a red LED) to indicate to the user that an error has occurred, or a message may be displayed on a display screen. In some examples, auto-injector 2 may need to be manually reset before an injection can be completed. In other examples, auto-injector 2 may loop back to step 2004, wherein controller 1408 continuously attempts to determine whether touch sensor 1410 is in contact with skin. Method 2000 also may require that touch sensor 1410 be in contact with skin during the entire injection. Thus, if at any point during the injection, controller 1408 determines that touch sensor 1410 is no longer in contact with skin, controller 1408 may stop the injection (e.g., by stopping further movement of translation mechanism 1366), may generate an error signal or message, and may retract needle 306 if it had been extended. By stopping the injection and retracting needle 306, a risk of dispensing the drug outside of the body (i.e. a wet injection) and/or needle stick injuries may be mitigated. Upon the determination of step 2004, auto-injector 2 may emit an audio tone and/or illuminate one or more LEDs to indicate that the auto-injector 2 is positioned on the skin surface. In one example, one or more additional LEDs of the first color may be illuminated at this stage to indicate further progress of the injection.

At step 2008, controller 1408 may send a signal to activate translation mechanism 1366. Once activated, translation mechanism 1366 may move toward second end 1306 of cartridge 1302 (referring to FIGS. 13 and 14), causing cartridge 1302 itself to move in the same direction. This may cause needle 308 to move in the opposing direction to access cartridge 1302 as set forth above. The movement of driver 1398 and needle 308 causes carrier 202 to move in the same direction, which sets forth the chain of events that ultimately deploys needle 306 into the user by the mechanisms set forth in FIGS. 5-11. Translation mechanism 1366 will continue to move toward second end 1306 until a desired amount of the drug contained within cartridge 1302 is dispensed into the user. Upon activation of the translation mechanism, auto-injector 2 may emit an audio tone and/or illuminate one or more LEDs to indicate that the injection is in progress. For example, yet additional LEDs of the first color may be illuminated as the injection progresses to give the user a visual indication of the progression.

Method 2000 may proceed to step 2010, where controller 1408 may determine whether the injection is complete. This determination may be based on interruption of beam 1430 by piston 1316 (as described with reference to FIGS. 4A, 13, and 14). That is, when beam 1430 is broken (not received by detector 1416), controller 1408 may determine that injection is complete. Once controller 1408 determines that the injection is complete, controller 1408 may send a signal to translation mechanism 1366 to reverse the direction of rotation of the lead screw, which may cause ramp 1500 to push against ramp 243 of stop 240, enabling retraction of needle 306 as discussed above with reference to FIG. 11. In one example, controller 1408 may institute a delay after receiving an indication that beam 1430 has been interrupted. The delay may be from, e.g., 0.1 to 60 seconds.

An additional end detection mechanism may be used instead of or in combination with the interruption-type sensor described above. For example, a current of the motor of translation mechanism 1366 may be utilized to determine whether an injection has been completed. That is, when piston 1316 reaches second end 1306 of cartridge 1302, the current on the motor will increase (e.g., as a result of piston 1316 engaging the end of cartridge 1302), signaling the expulsion of all or substantially all of the contents of cartridge 1302. One exemplary combination could include the use of beam 1430, where interruption of beam 1430 indicates that, e.g., 90 to 98 percent of the injection has been completed. Then, the current of the motor of translation mechanism 1366 could be analyzed to determine whether the remaining 2 to 10 percent of the injection has been completed. In another example, instead of using an optical switch, a delay from the initiation of the translation mechanism 1366 may be used by controller 1408 to determine when to reverse translation mechanism 1366. In one example, this delay may be from, e.g., about 1 to about 120 seconds, although other suitable times are also contemplated. In any event, the delay from initiation may be long enough to permit emptying of cartridge 1302. In still another example, beam 1430 may be used in combination with an encoder. The encoder may be configured to detect a position of piston 1316. If the encoder were used to detect the position of piston 1316 alone, a drive train issue could inhibit accurate detection. For example, piston 1316 may rotate when pushed by the lead screw. Such rotation may cause uncertainty as to actual position of piston 1316. When used in conjunction with beam 1430, however, controller 1408 may be configured to recalibrate the encoder in response to interruption of beam 1430. Such recalibration may allow controller 1408 to update the actual position of the encoder and resume accurate detection of the position of piston 1316 using the encoder.

Upon determination that the injection is complete, auto-injector 2 may emit an audio tone and/or illuminate one or more LEDs to indicate completion of the injection. In some examples, one or more LEDs of a second color (e.g., green) that is different from the first color may be illuminated to signal to the user that the injection is complete. In some examples, all of the LEDs of the device may be illuminated with the second color, and other indications also may be used. For example, all of the LEDs may be illuminated with the second color and may flash intermittently at the end of the injection.

In some examples, a timing of an injection procedure, measured from the initial activation of activating switch 1409 to retraction of needle 306 from the user after drug delivery, may be from about 20 seconds to about 90 seconds, or from about 25 seconds to about 60 seconds, from about 30 seconds to about 45 seconds, or less than or equal to about 120 seconds, or less than or equal to about 90 seconds, or less than or equal to about 60 seconds, or less than or equal to about 45 seconds, or less than or equal to about 30 seconds. Such timing represents a significant improvement over existing devices, for which the timing of an injection may be much longer and, in some cases, as long as about 9 minutes or even longer.

Method 2000 also may include additional steps. For example, method 2000 may include determining whether a drug within cartridge 1302 is too cold for delivery into the user, whether power source 1406 has enough energy to complete an injection, whether needle 306 has been prematurely deployed and/or retracted, whether the current of the motor of translation mechanism 1366 is in an appropriate range, and whether an injection procedure has extended beyond a maximum acceptable procedure time. When controller 1408 senses any of the above errors, it may communicate such errors to the user, and may end an ongoing injection by, e.g., halting or reversing translation mechanism 1366 and retracting needle 306 from the user. Auto-injector 2 may emit an audio tone and/or illuminate one or more LEDs indicative of any of the foregoing additional steps. For example, one or more LEDs of a third color (e.g., red) that is different than the first and second colors may be illuminated.

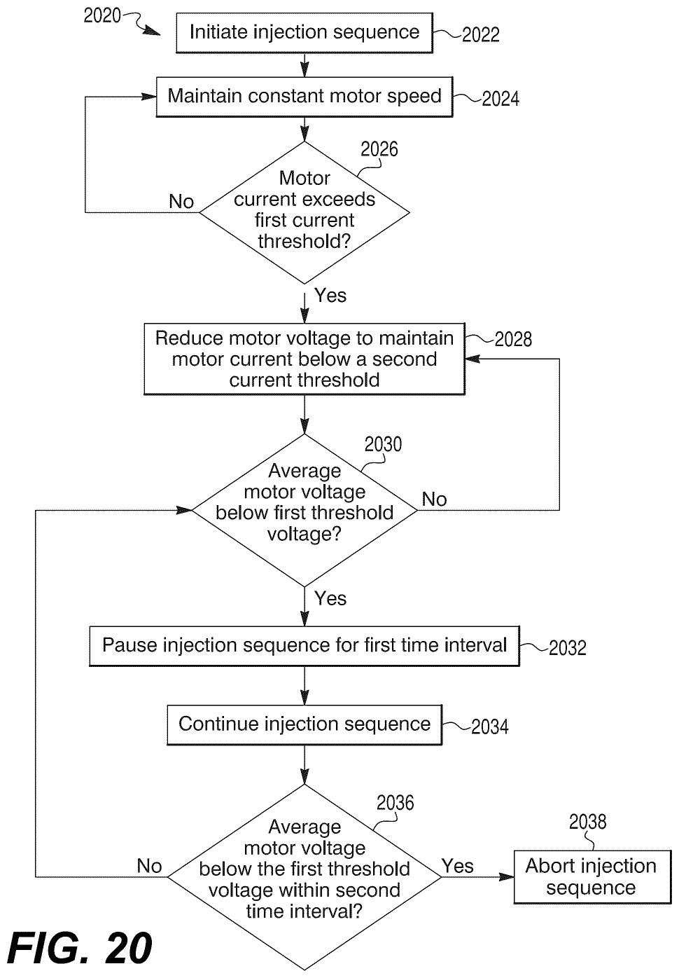

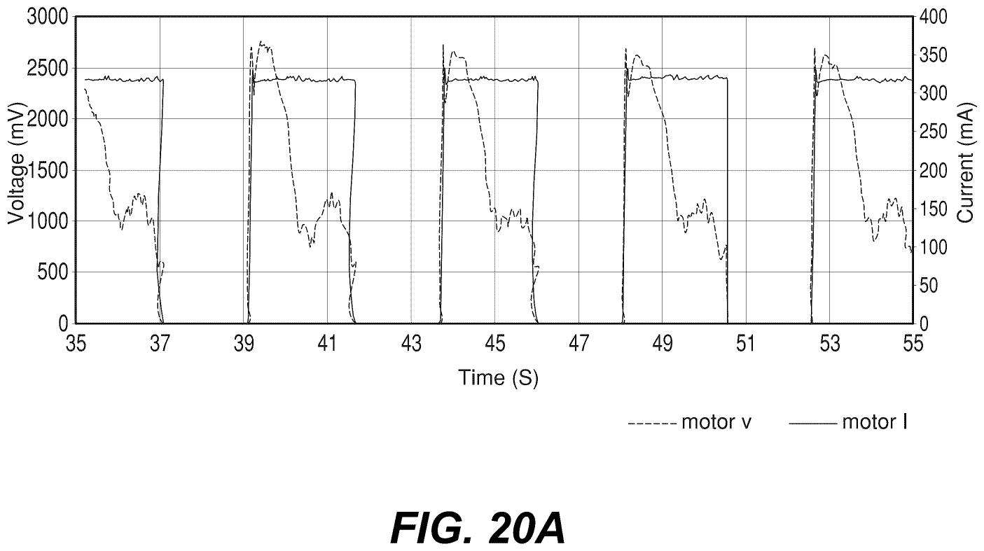

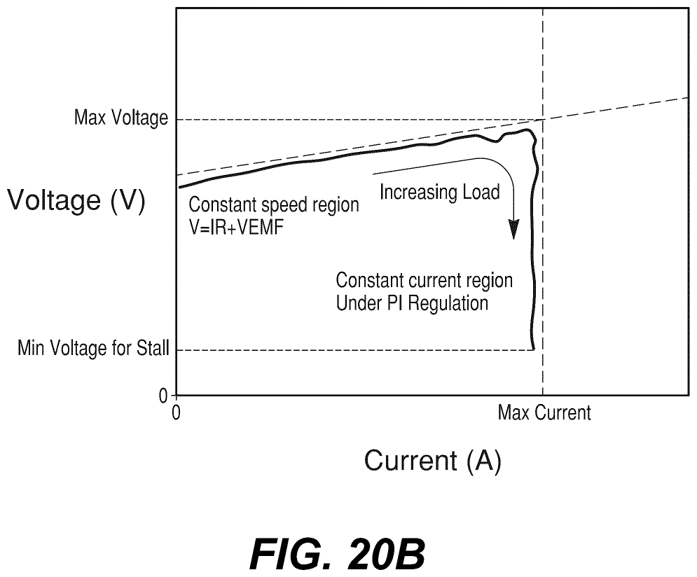

FIG. 20 shows an exemplary method 2020 of controlling a torque of the motor of translation mechanism 1366 and detecting when the motor stalls. At step 2022, controller 1408 may initiate an injection sequence. As described herein previously, an injection sequence may be initiated upon depressing activating switch 1409 against a user's skin and/or detecting the user's skin by touch sensor 1410. During the injection sequence, a voltage may be applied to the motor of translation mechanism 1366 to drive the motor.

At step 2024, as the injection sequence progresses, controller 1408 may maintain the motor of translation mechanism 1366 at a constant speed. The constant speed may be, for example, a rotational speed measured in revolutions per minute (RPM). Controller 1408 may maintain the motor at a constant speed by varying the voltage applied to the motor. For example, when a higher load is applied to the motor due to an obstruction, increased fluid pressure, increased component friction, or any other cause, controller 1408 may compensate for the increased load by increasing the voltage applied to the motor. Conversely, when a load applied to the motor is reduced, controller 1408 may compensate for the reduction in load by decreasing the voltage applied to the motor. Maintaining the motor at a constant speed may reduce a likelihood that the user experiences injection site pain. For example, maintaining the motor at a constant speed may prevent the bolus from become excessively large, thereby mitigating the risk of pain.

During the injection sequence, controller 1408 may monitor a current supplied to the motor. The motor current may be indicative of a torque generated by the motor. For example, a higher motor current may indicate a higher torque being generated by the motor. At step 2026, controller 1408 may determine whether the motor current exceeds a first current threshold. The first current threshold may be determined and/or set based on a maximum torque that may be safely generated by the motor. The maximum torque may be reached, for example, when the injection sequence is obstructed in some way. If controller 1408 determines that the motor current does not exceed the first current threshold, the method 2020 may revert to step 2024 and controller 1408 may continue to maintain the motor at a constant speed. If, on the other hand, controller 1408 determines that the motor current exceeds the first current threshold, method 2020 may proceed to step 2028.

At step 2028, controller 1408 may reduce the motor voltage to maintain the motor current below a second current threshold. In some embodiments, the second current threshold may be greater than the first current threshold and may more closely correlate to the maximum torque that may be safely generated by the motor. In some embodiments, the second current threshold may be less than, or the same as, the first current threshold. In the event that the injection sequence is obstructed, the motor speed may slow and the motor impedance may decrease. As the motor impedance decreases, a lower voltage may be required to maintain the motor current below the second current threshold. Controller 1408 may monitor an average motor voltage applied to the motor. The average motor voltage may be, for example, a time average.

Steps 2024 through 2028 of method 2020 may generally be illustrated by the graph depicted in FIG. 20B, in which a curve representing a relationship between the voltage applied to the motor of translation mechanism 1366 and the current consumed by the motor is plotted. The curve may be characterized by the following equation:

$$V = iR + {V}_{emf}$$

In the equation above, V is the voltage applied to the motor, i is a current consumed by the motor, R is a coil resistance of the motor, and V_{emf} is a back electromotive force that acts against the applied voltage at a given speed. As shown in FIG. 20B, the curve may include a constant speed region, in which the motor may be maintained a constant speed (step 2024). In the constant speed region, V_{emf} may remain approximately constant and the curve may be approximately linear.

As shown in FIG. 20B, as a load (i.e. a torque) acting on the motor of translation mechanism 1366 increases, the current consumed by the motor may increase. As the current approaches a Max Current, a voltage applied to the motor may be reduced, thereby maintaining the current below the Max Current (steps 2026 and 2028). The current consumed by the motor may be maintained below the Max Current using proportional integral (PI) regulation. As shown, there may be a minimum voltage for the motor below which the motor may stall.

Steps 2030 through 2038 of method 2020 may correspond to a control sequence for preventing stalling of the motor. FIG. 20A depicts a graph which may represent the voltage applied to the motor and the current consumed by the motor over time and in accordance with steps 2030 through 2038.

At step 2030, controller 1408 may determine whether the average motor voltage has decreased below a first threshold voltage. The average motor voltage decreasing below the first threshold voltage may indicate that the injection sequence is obstructed. If controller 1408 determines that the average motor voltage has not decreased below a first threshold voltage, method 2020 may revert to step 2028, at which controller 1408 may continue to maintain the motor current below the second current threshold. FIG. 20A illustrates five intervals during which controller 1408 may maintain the motor current at a constant value (e.g. below the second current threshold): between about 35 seconds and about 37 seconds, between about 39 seconds and about 41.5 seconds, between about 43.5 seconds and about 46 seconds, between about 48 seconds and about 50.5 seconds, and between about 52.5 seconds and about 55 seconds. As shown in FIG. 20A, the voltage applied to the motor during each interval may decrease, albeit with some fluctuations, to maintain the motor current below the second current threshold. Though the voltage during each interval in FIG. 20A is shown as decreasing, the voltage need not necessarily decrease to maintain the motor current below the second current threshold, but instead may stay flat in certain situations.

If, on the other hand, controller 1408 determines that the average motor voltage has decreased below the first threshold voltage, controller 1408 may cause the injection sequence to be paused for a first time interval. When causing the injection sequence to be paused, controller 1408 may cease applying voltage to the motor. In some embodiments, the first time interval may be 2 seconds, for example. FIG. 20A illustrates four such pauses: between about 37 seconds and about 39 seconds, between about 41.5 seconds and about 43.5 seconds, between about 46 seconds and about 48 seconds, and between about 50.5 seconds and about 52.5 seconds.

The first time interval may be sufficiently long to allow fluid pressure within auto-injector 2 to dissipate. The first time interval may also be sufficiently short such that the user may not be prompted to remove auto-injector 2 from the user's skin (e.g., the first time interval is set to be less than a typical reaction time of the user to falsely identify the end of the injection). The first time interval may further be indicated by illumination of one or more of the LEDs of the progress ring or another light within auto-injector 2 and visible by a user. The LEDs may be illuminated, for example, in a particular pattern or according to a particular color scheme to indicate the first time interval and that the injection sequence is paused rather than stopped.

After pausing the injection sequence, controller 1408 may continue the injection sequence at step 2034. To continue the injection sequence, controller 1408 may resume supplying voltage to the motor of translation mechanism 1366. At step 2036, controller 1408 may determine whether the average motor voltage has decreased below the first threshold voltage within a second time interval. The second time interval may be shorter than the first time interval and may be set and/or determined to be indicative of a confirmation that the injection sequence is obstructed. The second time interval may be, for example, about 0.9 seconds. If the motor voltage has not decreased below the first threshold voltage within the second time interval, method 2020 may revert to step 2030. If, on the other hand, controller 1408 determines that the motor voltage has decreased below the first threshold voltage within the second time interval, method 2020 may proceed to step 2038 at which controller 1408 may cause the injection sequence to be aborted.

In some embodiments, controller 1408 may perform step 2026 continuously as it performs steps 2028 to 2036. For example, controller 1408 may continue to determine whether the motor current exceeds the first current threshold as steps 2028 to 2036 are performed. If the motor current continues to exceed the first current threshold, method 2020 may proceed through steps 2028 to 2036 as described herein previously. In the event the motor current falls below the first current threshold, on the other hand, method 2020 may revert to step 2024 and controller 1408 may maintain the motor at a constant speed. In other words, if a high load on the motor, due to obstruction, high fluid pressure, or the like, dissipates during performance of steps 2028 to 2036, controller 1408 may simply revert to maintaining a constant motor speed rather than proceeding through any remaining steps unnecessarily.

Accordingly, method 2020 may allow controller 1408 to effectively distinguish between situations in which the needle may be partially blocked or a high friction force may be acting against the injection sequence, and situations in which the injection sequence is insurmountably obstructed. In the former situations, auto-injector 2 may have the ability to complete the injection sequence and the injection sequence may not be prematurely terminated. In the latter situations, auto-injector 2 may not have the ability to complete the injection sequence and the injection sequence may be appropriately terminated. In such situations, auto-injector 2 may emit an audio tone and/or illuminate one or more LEDs to indicate that the injection was terminated before completion. Method 2020 may further appropriately terminate an injection sequence in which the piston 1316 extends completely, indicating that the cartridge 1302 is empty. Method 2020 may further allow the auto-injector 2 to be used on an emergency basis if, for example, a user performs an injection without first warming up auto-injector 2 to decrease a viscosity of the medicament. Method 2020 may further allow an injection of a viscous medicament to proceed at a slower rate than the motor and gear reduction ratio may otherwise allow.

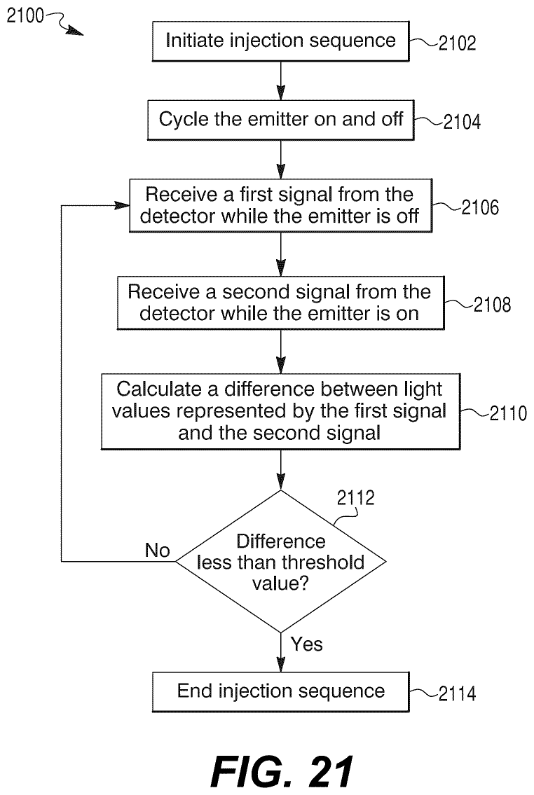

FIG. 21 shows an exemplary method 2100 of detecting an end of a dose of medicament using emitter 1414 and detector 1416. Method 2100 may be used, for example, to detect a time at which a full dose of medicament has been dispensed to a user and end the corresponding injection sequence.

At step 2102, controller 1408 may initiate an injection sequence. As described herein previously, an injection sequence may be initiated upon depressing activating switch 1409 against a user's skin and/or detecting the user's skin by touch sensor 1410. At step 2104, controller 1408 may cycle emitter 1414 on and off periodically. Emitter 1414 may be cycled on and off rapidly in a square wave pattern, such that emitter 1414 is turned off and on several times per second. Cycling emitter 1414 on and off may allow detector 1416 to be exposed to light produced by emitter 1414 in combination with ambient light, and also to ambient light alone.

At step 2106, controller 1408 may receive a first signal from detector 1416 corresponding to a time when emitter 1414 is off. The first signal may correspond to, and/or be indicative of, ambient light detected by the detector 1416. At step 2108, controller 1408 may receive a second signal from detector 1416 corresponding to a time when emitter 1414 is on. The second signal may correspond to, and/or be indicative of, light emitted by emitter 1414 in combination with ambient light as detected by the detector 1416.

At step 2110, controller 1408 may calculate a difference between a first light value represented by the first signal and a second light value represented by the second signal. The difference may be indicative of how much light detected by detector 1416 is attributable to light emitted by emitter 1414 as opposed to ambient light. At step 2112, controller 1408 may determine whether the difference is less than a threshold value. If controller 1408 determines that the difference is not less than a threshold value, method 2100 may revert to step 2106. If, on the other hand, controller 1408 determines that the difference is less than the threshold value, controller 1408 may end the injection sequence at step 2114.

Accordingly, method 2100 may be used to reduce the impact of ambient light when detecting an end of a dose of medicament. Specifically, method 2100 may address a situation in which light from emitter 1414 is blocked from reaching the detector 1416 indicating an end of a dose, yet ambient light is able to reach detector 1416 and create a false negative reading indicating that an end of dose has not been reached.

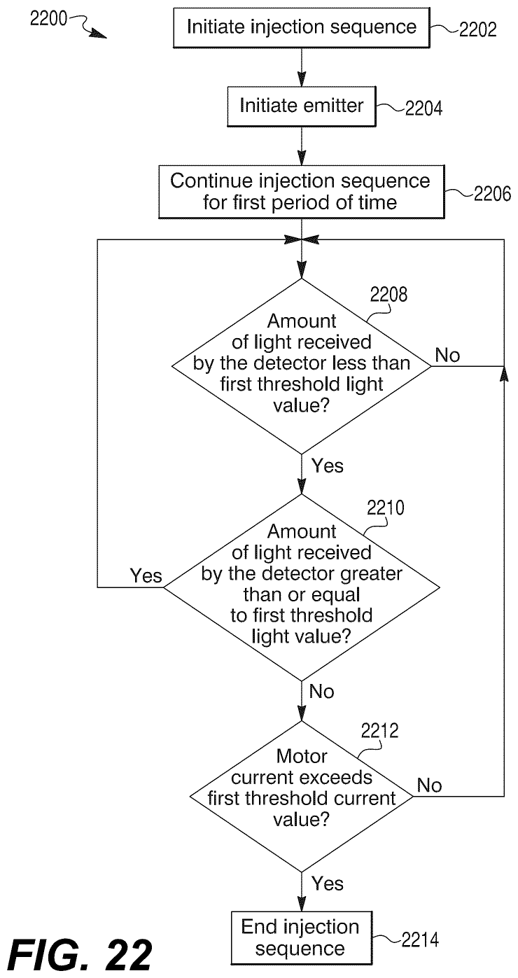

FIG. 22 shows another exemplary method 2200 of detecting an end of a dose of medicament using emitter 1414 and detector 1416. Method 2200 may be used, for example, to detect a time at which a full dose of medicament has been dispensed to a user and end the corresponding injection sequence.

At step 2202, controller 1408 may initiate an injection sequence. As described herein previously, an injection sequence may be initiated upon depressing activating switch 1409 against a user's skin and/or detecting the user's skin by touch sensor 1410. At step 2204, controller 1408 may initiate emitter 1414 or otherwise cause emitter 1414 to emit light.

At step 2206, controller 1408 may cause the injection sequence to continue for a first period of time. The first period of time may be a predetermined period of time corresponding to a duration in which a full dose cannot possibly be, or is unlikely to be, dispensed. For example, the first period of time may be between about 20% and 50% of the total injection time. During the first period of time, controller 1408 is not able to interrupt the injection sequence in response to a signal received from detector 1416 (but could still interrupt the injection sequence due to obstructions or stalling as discussed with reference to FIG. 20).

At step 2208, after the end of the first period of time, controller 1408 may determine whether an amount of light received by detector 1416 is less than a first threshold light value. Controller 1408 may make the determination based on a signal received from detector 1416 indicative of light received by detector 1416. The first threshold light value may correspond to an amount of light received by detector 1416 at the end of a dose. If controller 1408 determines that the amount of light received by detector 1416 is not less than the first threshold light value, controller 1408 may continue the injection sequence and method 2200 may otherwise remain at step 2208. If, on the other hand, controller 1408 determines that the amount of light received by detector 1416 is less than the first threshold light value, the method may proceed to step 2210.

At step 2210, controller 1408 may determine whether the amount of light received by detector 1416 is greater than or equal to the first threshold light value. If controller 1408 determines that the amount of light received by detector 1416 has risen to or above the first threshold light value, controller 1408 may continue the injection sequence and method 2200 may revert to step 2208. If, on the other hand, controller 1408 determines that the amount of light received by detector 1416 has remained less than the first threshold light value, the method may proceed to step 2212. Step 2210 may in effect enable controller 1408 to “clear” the injection sequence of anomalous interruptions of the light received by the detector, which may be caused by an air bubble within cartridge 1302 that blocks the path of light between emitter 1414 and detector 1416, for example, provided the amount of light subsequently meets or exceeds the first threshold light value.

At step 2212, controller 1408 may determine whether the motor current exceeds a first threshold current value. The first threshold current value may be determined and/or set based on a current indicative of an end of the injection sequence. The first current threshold value may be set, for example, based on a current indicative of piston 1316 reaching second end 1306 of cartridge 1302. If controller 1408 determines that the motor current does not exceed the first threshold current value, controller 1408 may continue the injection sequence and method 2200 may revert to step 2208. If, on the other hand, controller 1408 determines that the motor current exceeds the first threshold current value, method 2200 may proceed to step 2214 at which controller 1408 may cause the injection sequence to end.

Method 2200 may accordingly allow for accurate identification of the end of an injection sequence by identifying an instant in which both the light received by detector 1416 and the motor current are indicative of an end of the dose. By performing steps 2208, 2210, and 2212 sequentially, false identifications of the end of the dose due to either anomalous interruptions of light or anomalous high current events alone may be mitigated. Method 2200 may specifically reduce the impact of bubbles within cartridge 1302 on detection of the end of a dose of medicament.

FIG. 23 shows an exemplary method 2300 of operating activating switch 1409 of auto-injector 2 according to the disclosure. In particular, FIG. 23 depicts an exemplary sequence of positions of activating switch 1409 and corresponding functions of auto-injector 2.

Initially, at step 2302, auto-injector 2 may be disposed within a packaging such that plunger 1450 is in a depressed state and auto-injector 2 is in a low-power sleep mode. In some embodiments, during manufacturing auto-injector 2 may be programmed in an awake or active state. In some embodiments, if plunger 1450 is depressed for a predetermined period of time following programming, such as when auto-injector is placed in the packaging, auto-injector 2 may be configured to transition to the low-power sleep mode. The predetermined period of time may be any suitable period of time, such as 60 minutes, 30 minutes, 15 minutes, 10 minutes, 5 minutes, 2 minutes, or any other suitable period. Auto-injector 2 may be sealed in the packaging such that the packaging indicates that the auto-injector 2 has not been previously used. The packaging may be made from any suitable material, including paper, cardboard, plastic, cellophane, and the like. The packaging may press against plunger 1450 such that plunger 1450 is flush or nearly flush with housing 3 of auto-injector 2 and plunger 1450 is blocked from extending outwardly from auto-injector 2. With plunger 1450 in the depressed state, the circuit associated with activating switch 1409 may be open, thereby maintaining the auto-injector 2 in the low-power sleep mode.

At step 2304, auto-injector 2 may be removed from the packaging such that plunger 1450 is no longer depressed by the packaging and plunger 1450 may extend outwardly from the auto-injector 2. As plunger 1450 transitions from the depressed state to the free or extended state, plunger flange 1454 may contact or otherwise depress plunger switch 1448, thereby completing the circuit associated with activating switch 1409.

At step 2306, in response to the circuit associated with activating switch 1409 being completed, auto-injector may transition from the low-power sleep mode to an active mode. In the active mode, auto-injector 2 may calibrate touch sensor 1410. Auto-injector 2 may calibrate touch sensor 1410 by detecting a value or measurement of the touch sensor 1410 in ambient air, i.e. not against a user's skin. Auto-injector 2 may perform such calibration during a predetermined time period after auto-injector 2 is removed from the packaging (in some cases immediately after removal) so that such calibration occurs before a user may expose their skin to touch sensor 1410. In the active mode, auto-injector 2 may further detect whether emitter 1414 and/or detector 1416 are functioning properly, detect whether a needle is positioned properly, detect whether the motor of translation mechanism 1366 is responsive and/or operational, and/or perform any other suitable status tests. Auto-injector 2 may detect the positioning of the needle, for example, using a switch or detector configured to report the position of the needle to the controller 1408. In the active mode, auto-injector 2 may further illuminate one or more backlights to allow a user to inspect a vial and/or a drug contained in the vial through transparent window 50. In the active mode, auto-injector 2 may further display any other indication that auto-injector 2 is ready to be used.

At step 2308, auto-injector 2 may be placed against a user's skin such that plunger 1450 is depressed into auto-injector 2. Upon plunger 1450 being depressed, the circuit associated with activating switch 1409 may transition to an open state. As described above with reference to FIG. 18 and method 2000, auto-injector 2 may further detect contact with skin using touch sensor 1410. In response to depression of plunger 1450 and detection of contact with skin, auto-injector 2 may initiate an injection sequence at step 2310. The injection sequence may be a sequence resulting in injection of the user with a medicament, as described herein previously.

At step 2312, auto-injector 2 may be removed from the user's skin and plunger 1450 may again extend outwardly from auto-injector 2. Upon plunger 1450 extending outwardly, the circuit associated with activating switch 1409 may transition from the open state to the closed state. In response, auto-injector 2 may end the injection sequence at step 2310 and, for example, initiate retraction of the patient needle by reversing the motor. Auto-injector 2 may initiate retraction of the needle if the injection sequence has proceeded to completion or if the auto-injector has prematurely or accidentally been removed from the skin to prevent wet injection. Alternatively, in some embodiments, controller 1408 may determine whether a value received from touch sensor 1410 is indicative of the auto-injector 2 remaining in contact with the user's skin. If the value received by controller 1408 is indicative of the auto-injector 2 remaining in contact with the user's skin, auto-injector 2 may pause the injection sequence, thereby preventing wet injection. If the plunger 1450 is again depressed, thereby placing the circuit associated with activating switch 1409 in the open state, auto-injector 2 may resume the injection sequence.

According to the foregoing method 2300, activating switch 1409 may serve to keep auto-injector 2 in a lower-power sleep mode when in the packaging, transition auto-injector 2 to an active mode upon removal from the packaging, indicate when auto-injector 2 has been placed against a user's skin for an injection sequence, and indicate when auto-injector 2 has been removed from the user's skin at the end of an injection sequence. Moreover, a signal from activating switch 1409 may be cross-checked against a signal from touch sensor 1410 to more accurately determine whether auto-injector 2 has been removed from the user's skin, or whether, for example, an inadvertent or minor movement of auto-injector occurred.

### Data Collection and Transmission

FIG. 24 illustrates an exemplary schematic for data collection, storage, and transmission from auto-injector 2. Data may flow in both directions, and a healthcare provider (HCP) 1451 or clinical trials administrator 1453 may communicate with the patient 1447 or user 1449 of the mobile device 1407. Data may be stored locally on a non-volatile memory (NVM) of the auto-injector 2 or an NVM of the mobile device 1407, or may be stored remotely on, e.g., a cloud server. Dictionaries, language structures, and/or key-value pairs may be employed for data storage.

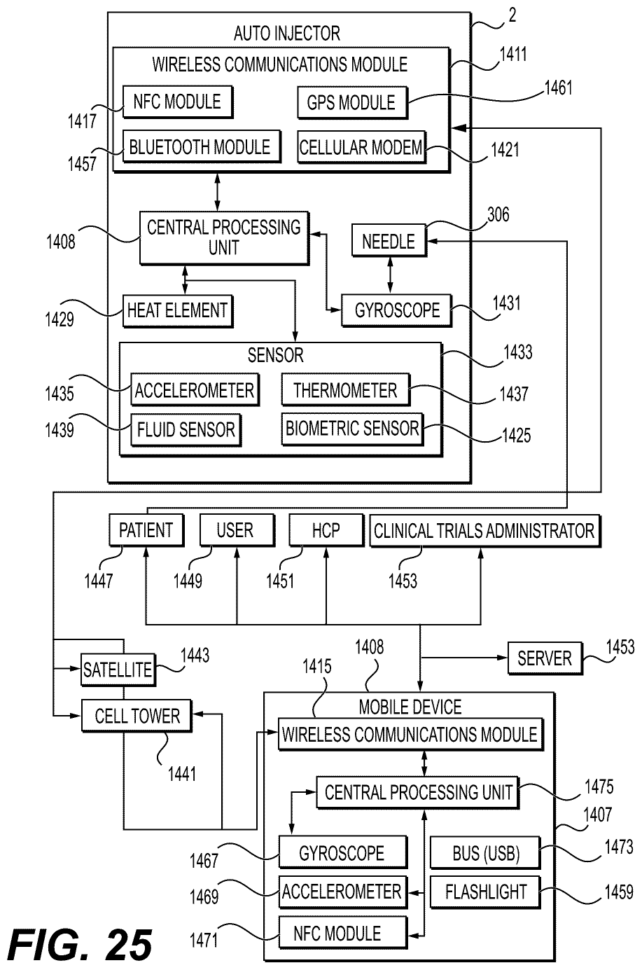

FIG. 25 shows a functional block diagram of auto-injector 2 and mobile device 1407 transmitting data between each other and from the mobile device 1407 to the patient 1447, the user 1449, the HCP 1451, and a clinical trials administrator 1453. For example, data collected by auto-injector 2 may be transmitted to a controller 1475 of the mobile device 1407 through a wireless communications module 1411 of the auto-injector 2. In particular, the auto-injector 2 may transmit data to mobile device 1407 through a wireless communications module 1411 of the auto-injector 2 to a wireless communications module 1415 of mobile device 1407 by any appropriate method, including through near-field communications (NFC) 1417 or Bluetooth modules 1457, WiFi modules 1419, communication via satellite 1443, and/or cellular modems 1421 which may communicate with a cellular tower 1441. Once the data is transmitted to the mobile device 1407 from the auto-injector 2, the data may be selectively transmitted through wireless communications module 1415 of the mobile device 1407 to the HCP 1451 or clinical trials administrator 1453, or any other appropriate recipient, as discussed fully herein. The mobile device 1407 may transmit data to the HCP 1451 or clinical trials administrator 1453 through an appropriate telecommunications medium, including by cellular tower 1441, satellite 1443, internet, WiFi, and/or remote server 1445. Once the data is sent to the HCP 1451 and/or clinical trials administrator 1453, the HCP 1451 and/or clinical trials administrator 1453 may aggregate all the data of a single patient 1447 or of multiple patients 1447 for analysis.

As previously mentioned herein, the controller 1408 of the auto-injector 2 may include a number of functionalities. The controller 1408 may be communicatively coupled to the wireless communications module 1411 and a sensor 1433. The controller 1408 may be configured to perform a variety of functions, including transmitting data, information, notifications, warnings, etc. to the mobile device 1407 through the wireless communications module 1411. As discussed herein, the controller 1408 may be configured to disable the needle 306 of the auto-injector 2 from being in the deployed configuration. This may occur automatically, or after receiving a command from, e.g., a controller 1475 of the mobile device 1407.

To disable the auto-injector 2, the plunger 1450 (FIGS. 3-3C) may be coupled to the housing 3 and movable relative to the housing 3. One or more electronics components may be used during an injection performed by the auto-injector 2, and may be formed within an electrical circuit. In a first configuration, a first portion of the plunger 1450 may be disposed within the housing 3 and the electrical circuit may be open. In such an implementation, the one or more electronics components may be in a low-power sleep mode. In a second configuration, the plunger 1450 may move outward relative to the housing 3, and the first portion of the plunger 1450 extends exterior of the housing 3. In the second configuration, the electrical circuit may be closed, and the one or more electronics components may be transitioned from the low-power sleep mode to an active mode. The plunger 1450 may be movable from the second configuration toward the housing 3 to a third configuration, and, in the absence of a separate instruction from the controller 1408, the auto-injector 2 may be configured to initiate an injection only after the plunger 1450 is moved to the third configuration and the one or more electronics components are in the active mode. However, depending on certain conditions, described herein, the auto-injector 2 may be configured to prevent initiation of an injection even after the plunger 1450 is moved to the third configuration. For example, if the medicament rises above a threshold temperature, as discussed herein, the auto-injector 2 may be disabled notwithstanding depressing the plunger 1450 in the third configuration.

Auto-injector 2 may include a number of additional electromechanical components. For example, auto-injector 2 may include a heat element 1429, which may include, e.g., a heating coil powered by a battery or some other power source. Heat element 1429 may selectively heat a medicament in cartridge 1302 based on a signal from controller 1408 of auto-injector 2. Once the signal is received from controller 1408, heat element 1429 may heat to a desired or optimal temperature associated with the medicament. Auto-injector 2 may be disabled until the medicament is warmed to the desired temperature. The heat element 1429 may be coupled to the controller 1408 of the auto-injector 2, and may take the form of a wire coil, a ribbon element, a tubular element, a resistive wire enclosed in ceramic or metal sheathing, a mica band, a thick film element, and/or a thermoelectric heat pump (also known as a Peltier device). Controller 1408 may raise or lower the temperature of the heat element 1429 by selectively powering the heat element 1429. The heat element 1429 may reach any appropriate temperature for warming the auto-injector 2 and/or the medicament therein. For example, the heat element 1429 may warm to temperatures between about 0° F. and about 32° F., about 32° F. and about 47° F., about 47° F. and about 59° F., 59° F. and about 77° F., 77° F. and about 98.6° F., about 68° F. and about 104° F., about 98° F. and 108° F., about 122° F. and about 140° F., about 122° F. and about 158° F., and up to about 212° F., based on the type of medicament. Factors affecting the temperature ranges may include, for example, protein stability, desired viscosity of the medicament, drug efficacy, and/or patient comfort. The controller 1408 may be configured to raise the temperature of the heat element 1429 while simultaneously monitoring the temperature of the heat element 1429 and/or the medicament with a thermocouple or thermometer 1437 on or within the housing 3 of the auto-injector 2. Once a threshold temperature is detected by the controller 1408 through the thermometer 1437, the controller 1408 may discontinue powering the heat element 1429. Although described as included within or on the auto-injector 2, the heat element 1429 may be, e.g., an add-on or third-party device.

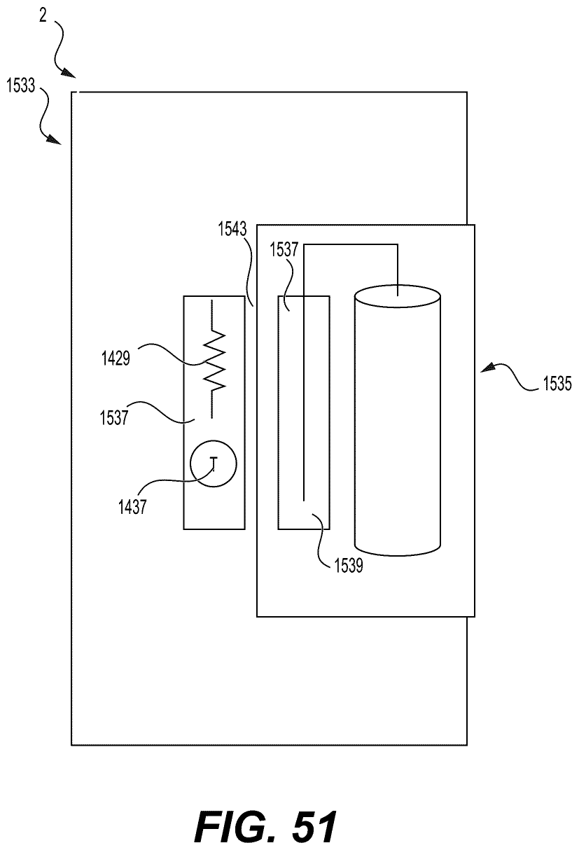

FIG. 51 shows the heat element 1429 within the auto-injector 2. The auto-injector 2 may include a thermal interface 1543 between a reusable portion 1533 of the auto-injector 2 and a disposable portion 1535 of the auto-injector 2. The disposable portion 1535 may include, e.g., cartridge 1302, translation mechanism 1366, needle 308, and fluid path 308c, while the reusable portion 1533 may include all other components. However, both disposable and reusable portions 1535 and 1533 each may include any components described herein. The disposable portion 1535 may have a thermally conductive block 1537 to transfer heat from the interface 1543 to a fluid path. The reusable portion 1533 may include the heat element 1429 and sensor (e.g., a thermometer 1437, discussed herein) on a block 1539 to transfer heat to the interface 1543. The interface 1543 may include a compliant thermal interface material (e.g., grafoil, braided metal, filled rubber) between the block 1539 of the reusable portion 1533 and a block 1537 of the disposable portion 1533.

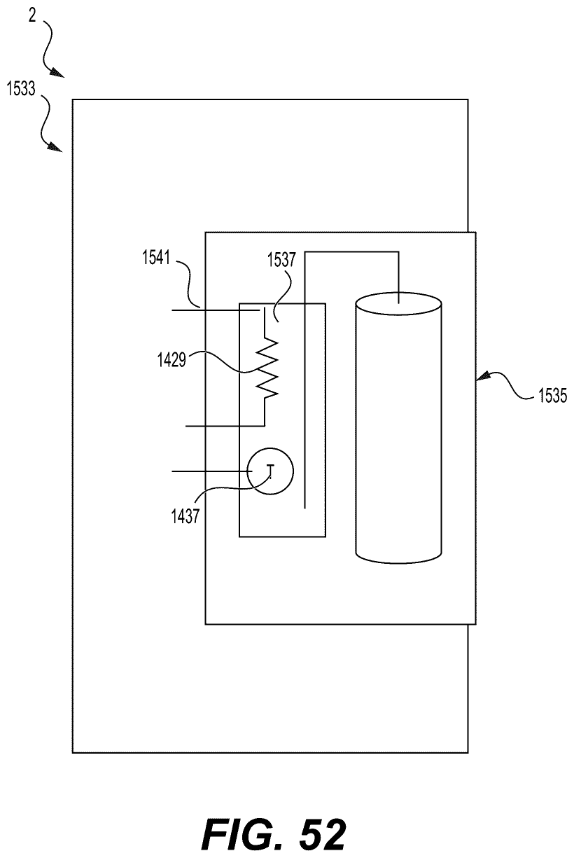

FIG. 52 shows a substantially similar embodiment as FIG. 51 except that the disposable portion 1535 includes the heat element 1429, thermometer 1437, and thermally conductive blocks 1537 and 1539. An electrical contact interface 1541 between the reusable and disposable portions 1533 and 1535 may be engaged when the cassette (e.g., the disposable portion 1535) is installed within the auto-injector 2. Either portion 1533 and 1535 may include the blocks 1537 and 1539.

As mentioned previously, the auto-injector 2 may include a thermocouple or thermometer 1437 on or within housing 3 to track the temperatures of multiple structures within the auto-injector 2, including the medicament, the electronics of the auto-injector 2, and/or the injection site of the patient. Thermometer 1437 may include a digital thermometer coupled to the controller 1408 of the auto-injector 2. The thermometer 1437 may be communicatively coupled to the controller 1408 by wired connection or wirelessly. The controller 1408 may be configured to compare measured temperatures to predefined and/or expected ranges of temperatures. As an example, the temperature ranges of a medicament may be between about −320° F. and about −238° F., about −238° F. and about 32° F., about 32° F. and about 47° F., about 47° F. and about 59° F., about 59° F. and about 77° F., about 68° F. and about 77° F., about 59° F. and about 86° F., about 77° F. and about 98.6° F., about 95° F. and about 104° F., about 98.6° F. and about 212° F., and above 212° F. If a temperature of the medicament is measured by the controller 1408 through the thermometer 1437 is below the predetermined threshold, the controller 1408 may initiate warming of the medicament through heat element 1429. As another example, the temperature ranges of the injection site may be about 95.0° F. to 99.0° F. The user 1449 may initiate the warming sequence or the warming sequence may initiate automatically. For example, the warming sequence may initiate when the button at the bottom of the auto-injector 2 is depressed, or, when warming is estimated to be quick, in response to the user 1449 pressing a button on the mobile device 1407 to initiate the warming sequence. Alternatively, or in addition, the warming sequence may automatically initiate when the auto-injector 2 is removed from the manufacturing packaging. If the injection site reaches a predefined maximum temperature, controller 1408 of auto-injector 2 may disable auto-injector 2. Alternatively, auto-injector 2 may transmit temperature data to mobile device 1407 through their respective wireless communications modules 1411 and 1415 for comparison. Based on that comparison, auto-injector 2 may receive a disable command from mobile device 1407 disabling fluid conduit 300 of auto-injector 2 from being in the deployed configuration until a second command from mobile device 1407 and overriding the first command is received by auto-injector 2. In some implementations, thermometer 1437 may be configured to detect fluids leaking in auto-injector 2 by, e.g., a rapid rise or decrease in temperature that may indicate a leak. Although described as included within or on the auto-injector 2, the thermometer 1437 may be, e.g., an add-on or third-party device.

Auto-injector 2 may include a fluid sensor 1439 within or coupled to housing 3 and communicatively coupled to controller 1408. Fluid sensor 1439 may include any appropriate fluid detection sensor, including a level sensor, pressure sensor, temperature sensor, pH sensor, conductivity sensor, turbidity sensor, chemical composition sensor, a destructive pin moisture sensor, a non-destructive moisture sensor, a paper moisture sensor, and/or a viscosity sensor. Fluid sensor 1439 may include a sensing element that may interact with the detected fluid and may include a pressure-sensitive diaphragm or bellows, thermistors, electrodes for pH or conductivity sensors, and/or floats. Fluid sensor 1439 may include a transducer to convert the physical property of the detected leak into an electrical signal, including a strain gauge, a thermoelectric device, or a set of electromagnetic coils. Fluid sensor 1439 may be communicatively coupled to controller 1408 of auto-injector 2 by a wire or wirelessly. If a leak is detected, controller 1408 of auto-injector 2 may send a leak detected signal to mobile device 1407 through wireless communications module 1411 of auto-injector 2. Once a leak-detected signal is received by mobile device 1407, mobile device 1407 may be configured to display a notification to the user and/or patient of auto-injector 2 or disable auto-injector 2. Although described as included within or on the auto-injector 2, the fluid sensor 1439 may be, e.g., an add-on or third-party device.

Auto-injector 2 and/or mobile device 1407 may include a biometric sensor 1425. The biometric sensor 1425 may sense the biometrics of the user of the auto-injector 2 to verify the user is an authorized user. Auto-injector 2 may include biometric sensor 1425 on or within housing 3. Biometric sensor 1425 may be any appropriate biometric sensor, including a fingerprint sensor, a facial recognition sensor, an iris or retina sensor, a voice recognition sensor, or palm vein sensor. Although described as included within or on the auto-injector 2, the biometric sensor 1425 may be, e.g., an add-on or third-party device.

Auto-injector 2 may include a gyroscope 1431. The gyroscope 1431 may include an optical gyroscope or a microelectromechanical gyroscope, and may be used to detect the orientation and/or angular velocity of the auto-injector 2. In some implementations, the gyroscope 1431 may be used to detect if the auto-injector 2 was rotated previously and replaced in its original packaging, thereby determining whether the auto-injector 2 may have been tampered with. As another example, the gyroscope 1431 may be used to determine, e.g., an angle and/or orientation of the auto-injector 2 during an injection sequence. Although described as included within or on the auto-injector 2, the gyroscope 1431 may be, e.g., an add-on or third-party device. Auto-injector 2 may include the accelerometer 1435. The accelerometer 1435 may include or take the form of any appropriate accelerometer device including a microelectromechanical device, a piezoelectric device, a capacitive device, a piezoresistive device, a servo device, or a fiber optic device. Accelerometer 1435 may be configured to determine or infer the orientation of the user 1449 based on an orientation of the auto-injector 2. Although described as included within or on the auto-injector 2, the accelerometer 1435 may be, e.g., an add-on or third-party device.

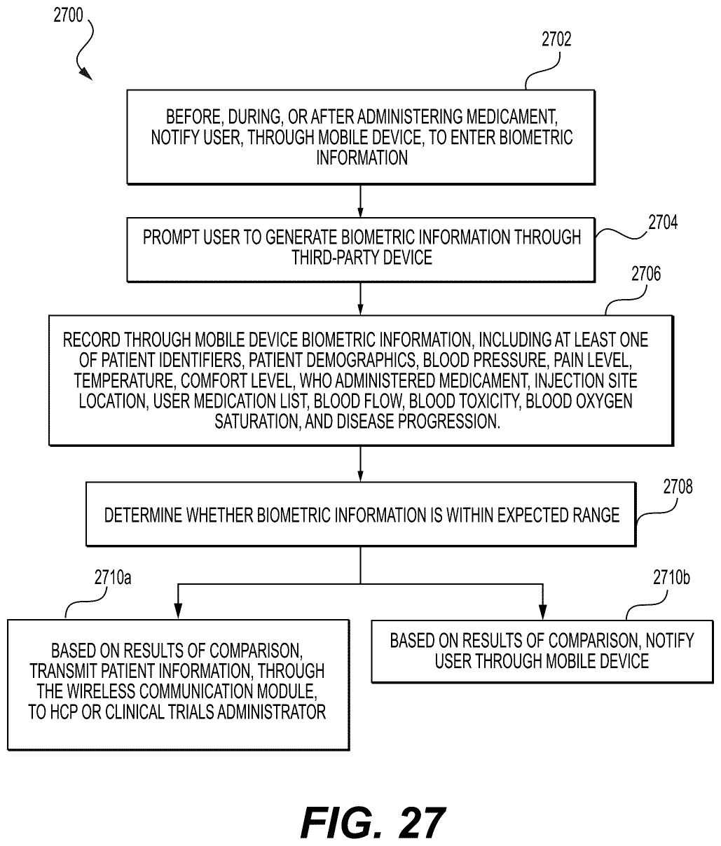

FIG. 26 shows, at a high level, a non-exhaustive list of the different types of data auto-injector 2 may transmit to mobile device 1407 through wireless communications module for data tracking and analysis, while FIGS. 27-50 show exemplary methods of data auto-injector 2 and mobile device 1407 may track. For example, the auto-injector 2 may collect biometric data 1501, patient identification information 1503, temperature data 1505, injection force data 1507, needle depth data 1509, injection data 1511, injection frequency data 1513, charge state 1515, leak data 1517, location data 1519, data on an error state 1521, and/or inventory data 1523 of the auto-injector 2 and/or cartridge 1302, as discussed herein. Additionally, any of the aforementioned data may be collected by a third-party device, such as a blood pressure cuff, and input into mobile device 1407. Although only some types of data are illustrated, any appropriate data may be collected and/or transmitted through auto-injector 2 to mobile device 1407. Also in FIG. 26 is a non-exhaustive list of actions the mobile device 1407 can take, including disabling the auto-injector 2, notifying a healthcare provider (HCP) 1525, notifying a clinical trials administrator 1527, and notifying a user 1529 of the data and various aspects of the data, including trends, discrepancies, disparities, etc. For example, data may be collected and transmitted to an HCP 1525 for tracking in-office and for statistical analysis. As another example, data may be collected and transmitted to the clinical trials administrator 1527 for clinical study tracking, e.g., participant compliance with a medicament administration regimen. Clinical studies tracking, for example, may include updating the file of a given patient 1447 in real time. In some implementations, all tracked auto-injectors 2 may be studied and the data aggregated and cross-referenced and analyzed for trends.

Referring to FIG. 27, which shows an exemplary method 2700 of recording patient biometric information. Method 2700 may include step 2702 prompting a user, before, during, or after administering a medicament through auto-injector 2, and through mobile device 1407, to enter biometric information of the patient into mobile device 1407. In one embodiment, method 2700 may include optional step 2704, prior to entering the biometric information, measuring the biometric information through a third-party device. Method 2700 may further include step 2706, including recording, through mobile device 1407, the biometric information. Biometric information may be recorded by either the auto-injector 2 automatically transmitting biometric information to the mobile device 1407, or by a user manually inputting the biometric information into mobile device 1407. Here, the biometric information may include any appropriate biometric, including, for example, patient identifiers; patient demographics; blood pressure; heart rate; pain level of the patient; temperature (of, e.g., the injection site); comfort level of the patient; the identity of the user who administered the medicament to the patient; injection site; a patient medication list; blood flow; blood toxicity; blood oxygen saturation; and disease progression and symptoms of a disease of the patient.

Patient identifiers, for example, may include an appropriate identifier, including name, date of birth (DOB), age, disease, fingerprint, and face scan. Patient demographics may include, for example, patient age, sex, gender identity, sexual history, education, address, nationality, ethnicity, marital status, family status, household income, employment status, and/or religion. Blood pressures may include, for example, a systolic over diastolic reading. Heart rates may include, for example a number of beats per minute. A pain level may include an arbitrary pain scale of 1 to 10 that the patient or user inputs through mobile device 1407. Temperature data may include a patient temperature (e.g., 98.6 degrees fahrenheit) or a temperature of the injection site. The body temperature of the patient and/or the body temperature of an injection site may be collected through a temperature sensor 1437 of the auto-injector 2. A comfort level of the patient may include a mood (e.g., happy, sad, or nervous) or arbitrary level of comfortability (e.g., on a scale from 1 to 10) of the patient. Patient hesitations may be tracked. The identity of the user who administered the medicament to the patient may include, e.g., a nurse, doctor, family member, etc. The injection site may include, for example, the type of injection (e.g., intramuscular or subcutaneous), or the body part at which the injection was administered (e.g., thigh, belly, or shoulder). A patient medication list may include, for example, patient medications taken in addition to the medicament (e.g., insulin for diabetics) and may be cross-referenced by the controller 1408 of the auto-injector 2 and/or the mobile device 1407 for interactions. Blood flow data may include a narrative or picture (taken via the mobile device 1407) of a type or amount of blood flow at the injection site after administering the medicament. Blood flow data may be collected, inferred, and/or estimated from regularly available data, including data collected by, e.g., a mobile device 1407 (e.g., a smartphone, smartwatch, headset, and/or any other medical monitoring device). Depending on the blood flow data collected, an HCP 1451 and/or clinical trials administrator 1453 may determine whether the blood flow is normal (e.g., healthy) or abnormal or pathological. Blood toxicity data may include an amount of a substance in the patient's blood, or it may include an estimation or inference of blood toxicity based on the amount of medicament administered and other biometric information (e.g., age, weight, medication list, and disease progression). Blood oxygen saturation data may include the fraction of oxygen-saturated hemoglobin in the patient's blood. Disease progression of a disease of the patient may include an appropriate characterization of a patient's disease. In one example, the disease progression data may include a staging of cancer (e.g., stages 1-4). In other example, disease progression may identify another characteristic of a disease, such as beginning, advanced, progressive, in remission, or cured. User 1449 disease progression may be tracked as a function of treatment (e.g., did progression slow or speed up after treatment). Symptoms may be tracked for, e.g., adverse events associated with medicament administration. Symptoms may be tracked, e.g., through a mobile application of the mobile device 1407.

Method 2700 may include step 2708, including determining whether the biometric information is within an expected range. An expected range may be a range that is, e.g., predicted by patient demographics (e.g., age, weight, etc.), disease progression, or prior history. The expected range of blood pressures may include an appropriate systolic over diastolic reading, including, for example, 120 mmHg over <80 mmHg, 120-129 mmhg over <80 mmHg, 130-139 mmHg over 80-89 mmHg, >140 mmHg over >90 mmHg, and/or >180 mmHg over >120 mmHg. Comparing the measured or entered biometric data with the expected biometric data may include any appropriate comparison method, including direct comparison, employing sorting and searching algorithms, hashing the data, comparing values with relational operators, data visualization tools, and/or various statistical methods, including t-tests, ANOVA, correlation coefficients, etc.

Method 2700 may include any number of actions based on whether the biometric information is with the expected range. For example, method 2700 may include, based on whether the biometric information is within the expected range, alternative step 2710a, including notifying the user through the mobile device that, e.g., the biometric information is outside of the expected range, including whether the incorrect biometric information was input. As another example, if the disease progression is beyond a predetermined threshold or if a given injection site has been used too many times, method 1000 may include recommending a new injection site. Method 1000 may include analyzing the biometric information for trends with a machine learning algorithm and notifying the user, through mobile device 1407, of the trends. Step 2710a may include disabling auto-injector 2 by disabling fluid conduit 300 of auto-injector 2 from being in a deployed configuration associated with administering the medicament.

Method 2700 may include, alternative step 2710b, including transmitting the biometric information to an HCP or a clinical trials administrator. Transmitting the biometric information may include transmitting the biometric information through a remote server or any appropriate data transmission method (e.g., NFC, Bluetooth, WiFi, cellular modem, or satellite).

In some embodiments, the mobile application may be configured to authenticate a user of the auto-injector 2 prior to use. For example, the mobile application, in connection with the user's mobile device, may use biometric identification, two-factor authentication, or any other suitable authentication protocol to confirm the identity of the user prior to an injection. Upon authentication of the user, the mobile application may cause the auto-injector to become activated or otherwise be unlocked. Such user authentication may inhibit misuse and/or waste of costly medicaments by persons other than an intended or authorized user.

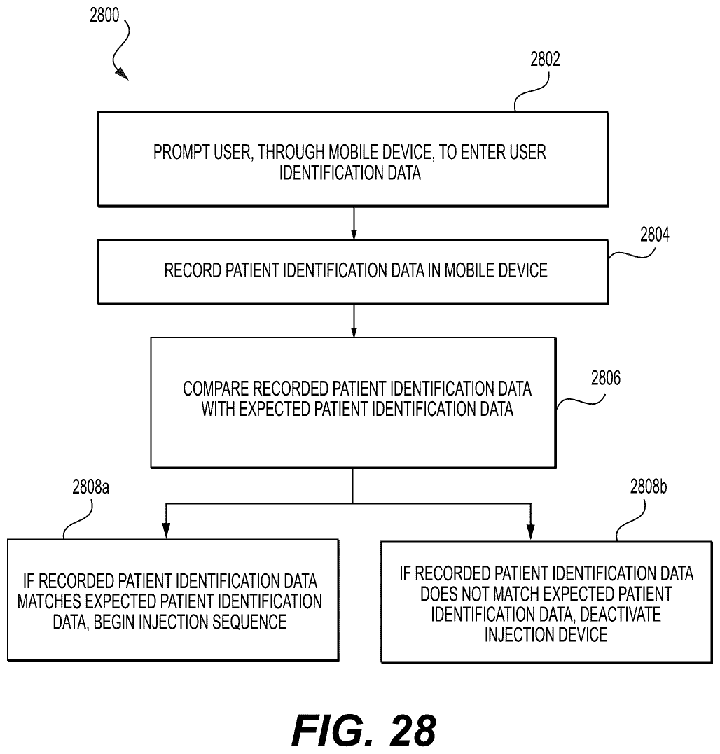

FIG. 28 shows an exemplary method 2800 for verifying a patient's identity. Method 2800 includes step 2802, including prompting a user, prior to administering a medicament through auto-injector 2 or through mobile device 1407, to enter patient identification information. Here, the user may be the patient or a third party (e.g., a nurse, doctor, family member). Patient identification information may include any appropriate patient identifiers, as discussed previously (e.g., name, DOB, age, fingerprint, face scan, etc.). The user may enter the patient identifiers through mobile device 1407 by, e.g., typing the identifiers into the mobile device 1407. Alternatively, the user may verify the patient's identity by using a biometric sensor 1425, e.g., a fingerprint scanner and/or face scanner, of mobile device 1407. Method 2800 may also include optional step, including recording or logging the entered patient identification information for future reference.

Method 2800 may include step 2806, including comparing the entered patient identification information with expected patient identification information. Expected patient identification information may include predetermined identification data, such as a patient's name, DOB, and/or a unique patient identification number, a QR code, and/or a barcode associated with the patient 1447 (e.g., on a medical bracelet). In some implementations, an HCP may set the expected patient identifiers remotely. In other implementations, the expected patient identifiers may be set by a user or patient to preclude others from operating auto-injector 2. In yet other implementations, a user 1449 or an HCP 1451 may scan an QR code or barcode to assign the auto-injector 2 to a given patient 1447. Thereafter, the patient 1447 may have to scan the QR code or barcode associated with that patient 1447 to use the auto-injector 2.

Method 2800 may include step 2808a, including, based on the comparison of the entered patient identification information and the compared patient identification information, initiating administration of the medicament. That is, once the identity of the patient is verified, auto-injector 2 may begin its administration sequence.

Method 2800 may include an alternative step 2808b, including, based on the comparison of the entered patient identification information and the compared patient identification information, disabling auto-injector 2. Step 2808b may include disabling the auto-injector by disabling fluid conduit 300 of auto-injector 2 from being in the deployed configuration associated with administering the medicament.

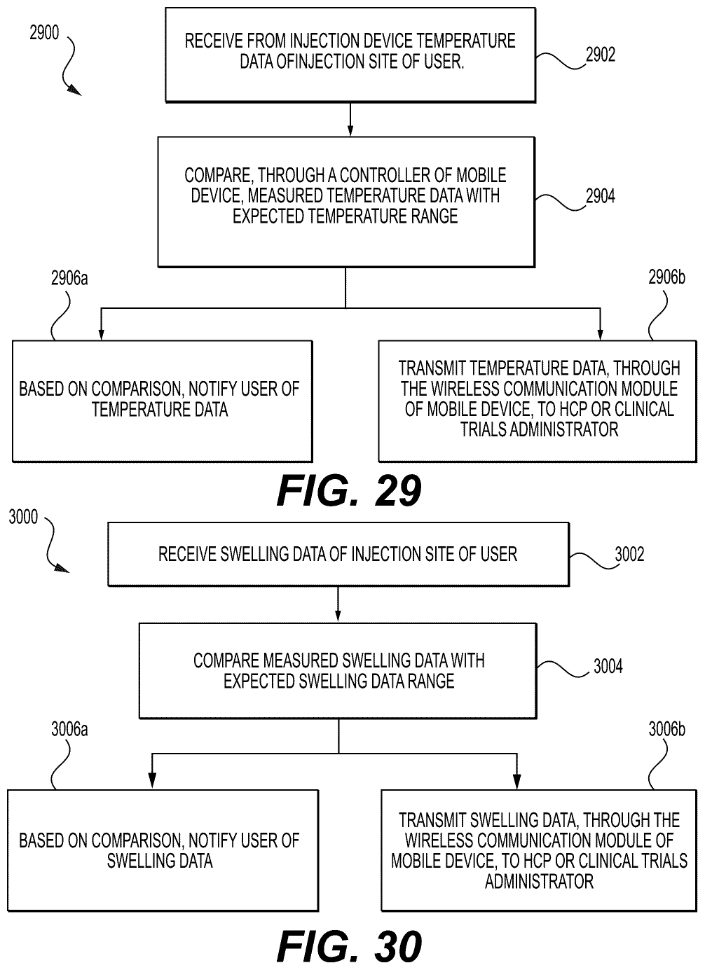

FIG. 29 shows an exemplary method 2900 for collecting data associated with the temperature of an injection site of the patient. Step 2902 may include receiving, from auto-injector 2, measured temperature data of an injection site (e.g., the thermal radiation at the injection site) of the patient of auto-injector 2. The temperature data may be measured through a temperature sensor (e.g., thermometer 1437) on housing 3 of auto-injector 2.

Method 2900 may include step 2904, including comparing the measured temperature data with expected temperature data. Step 2904 may include comparison methods described herein, e.g., direct comparison, statistical comparisons, etc.

Method 2900 may include step 2906a, including, based on the comparison of the temperature data and the expected temperature data, notifying the user of the temperature data. If the temperature data is equal to or around the threshold temperature, method 2900 may include taking no action. If the temperature data exceeds a threshold temperature, method 2900 may include initiating cooling systems, open valves to release hot fluids, trigger an alarm or warning lights, sever power to parts or all of the auto-injector 2, engage heat sinks, or disable and/or power down the auto-injector 2. If the temperature data is below a threshold temperature, method 2900 may include activating heating systems (e.g., heat element 1429), closing valves to retain heat and/or prevent cold fluid ingress, trigger alarms and/or warning lights, increase power to components of the auto-injector 2, and/or disable or power down the auto-injector 2. The temperature data may be measured in real time while administering a medicament through auto-injector 2. Additionally, notifying the user may include notifying the user of the temperature of the device in real time.

Method 2900 may include step 2906b, including transmitting the temperature data, through a wireless communications module of the mobile device, to a healthcare provider or a clinical trials administrator. Transmitting the temperature data may include transmitting the biometric information through a remote server or any appropriate data transmission method (e.g., NFC, Bluetooth, WiFi, cellular modem, or satellite) of mobile device 1407.

FIG. 30 shows an exemplary method 3000 of notifying a user of excessive swelling at an injection site. Method 3000 may include step 3002, including receiving swelling data of an injection site of a patient. This may be received in real time (e.g., as auto-injector is administering the medicament) or anytime thereafter. Swelling data may include data associated with the temperature and/or curvature of the injection site through, e.g., a pressure sensor. An increase in temperature at the injection site, for example, may indicate increased blood flow to the injection site due to swelling.

Method 3000 may include step 3004, including comparing measured swelling data with an expected swelling data range. Step 3004 may include comparison methods described herein, e.g., direct comparison, statistical comparisons, etc. Method 3000 may include step 3006a, including, based on the comparison of the measured swelling data and the expected swelling data range, notifying the user and/or patient of the swelling data. The notification may include a summary of the swelling data, trends, discrepancies and/or disparities between the measured and expected data. For example, the notification may indicate the temperature of the injection site to the user 1449. The notification may include audio feedback or a visual warning (e.g., a notification on mobile device 1407). Method 3000 may include step 3006b, including transmitting the swelling data to an HCP or clinical trials administrator. Transmitting the swelling data may include transmitting the swelling data wirelessly to a computing device of the HCP or clinical trials administrator.

In yet other implementations, if the swelling data indicates severe swelling, method 3000 may also include disabling the auto-injector 2 from use notwithstanding the user attempting to use auto-injector 2. Disabling auto-injector 2 may include a controller of the mobile device disabling a fluid conduit of the auto-injector from being in a deployed configuration.

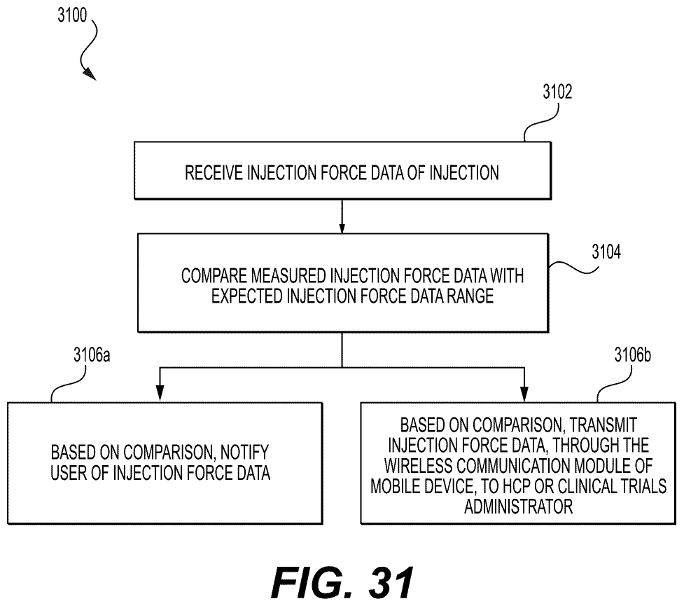

FIG. 31 shows an exemplary method 3100 of comparing injection force data of auto-injector 2. Method 3100 may include step 3102, including receiving, from auto-injector 2, injection force data collected through a sensor 1427 on housing 3 of auto-injector 2. The injection force data may be measured through, e.g., a sensor 1427 of auto-injector 2.

Method 3100 may include step 3104, including comparing, using a controller 1455 of mobile device 1407, the injection force data to expected injection force data. Step 3104 may include comparison methods described herein, e.g., direct comparison, statistical comparisons, etc.

Method 3100 may include step 3106a, including, based on the comparison of the injection force data to expected injection force data, notifying a user of auto-injector 2 of a disparity between the injection force data and the expected injection force data. The notification may be through any appropriate discussed herein, e.g., a text notification, audio feedback, etc. The notification may include, for example, the injection force of the last injection (e.g., 1 megapascals (MPa), 2.5 MPa, 4 MPa, 6 MPa, etc.), an amount of medicament injected, and/or a table including historical injection force data points with corresponding injection sites. Method 3100 may optionally include giving the user an option to log the injection force data for further analysis and/or record keeping. Notifying the user of auto-injector 2 of the compared injection force data may further include, e.g., recommending a different injection site to the patient. The recommendation may be through any appropriate means, including a text or audio guidance. In some implementations, the recommendation may include displaying instructions on mobile device 1407 to guide the patient and/or user to a different injection site.

Method 3100 may include step 3106b, including, based on the comparison of the injection force data, transmitting the injection force data, through a wireless communication module 1415 of the mobile device 1407, to an HCP or clinical trials administrator.

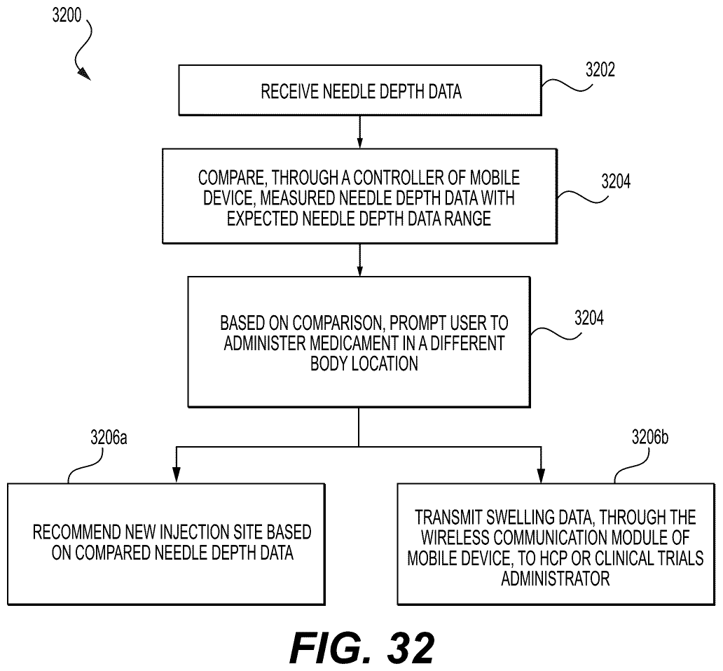

FIG. 32 shows an exemplary method 3200 of measuring needle depth data during injection. Method 3200 may include step 3202, including receiving needle depth data associated with the depth of needle 306 of auto-injector 2 penetrates an injection site of a user. The needle depth data may be collected while administering a medicament using auto-injector 2. Step 3202 may also include measuring, e.g., an angle at which needle 306 is inserted at the injection site.

Method 3200 may include step 3204, including comparing, through controller 1455 of mobile device 1407, the needle depth data with expected needle depth data. Step 3004 may include comparison methods described herein, e.g., direct comparison, statistical comparisons, etc. The expected needle depth data may differ depending on type of injection (e.g., subcutaneous or intramuscular). For subcutaneous injections, the expected needle depth data may range from less than 6 millimeters (mm) to about 13 mm. For intramuscular injections, the expected needle depth data may range from less than 16 mm to about 25 mm, depending on the age of the patient. In one example, if the needle depth is below a threshold value (e.g., the needle 306 does not penetrate deep enough into the injection site) or above a threshold value (e.g., the needle 306 is penetrating too deep in the injection site), then the mobile device 1407 may notify the user 1449 of a poor or failed injection, terminate the injection, automatically retract the needle 306, display a notification to change the needle 306, recommend a new injection site, and/or disable the auto-injector 2. The notification may include an injection depth data point, or a table of historical injection depth data points with corresponding injection sites.

Method 3200 may include step 3206 based on a comparison of the needle depth data with the expected needle depth data, notifying the user 1449 of the injection depth data. In one embodiment, method 3200 may include optional step 3208a, including recommending a new injection site to the user if the needle depth data exceeds or falls below a threshold value by too much. Method 3200 may optionally include modifying a length needle 306 exits housing 3 of auto-injector 2 while injecting the medicament. For example, the needle 306 may exit the housing 3 more or less depending on the amount the carrier 202 slides between the first and second positions.

Method 3200 may include step 3208b, including transmitting, through wireless communications module 1415 of mobile device 1407, the needle depth data to an HCP or clinical trials administrator.

### Injection Data

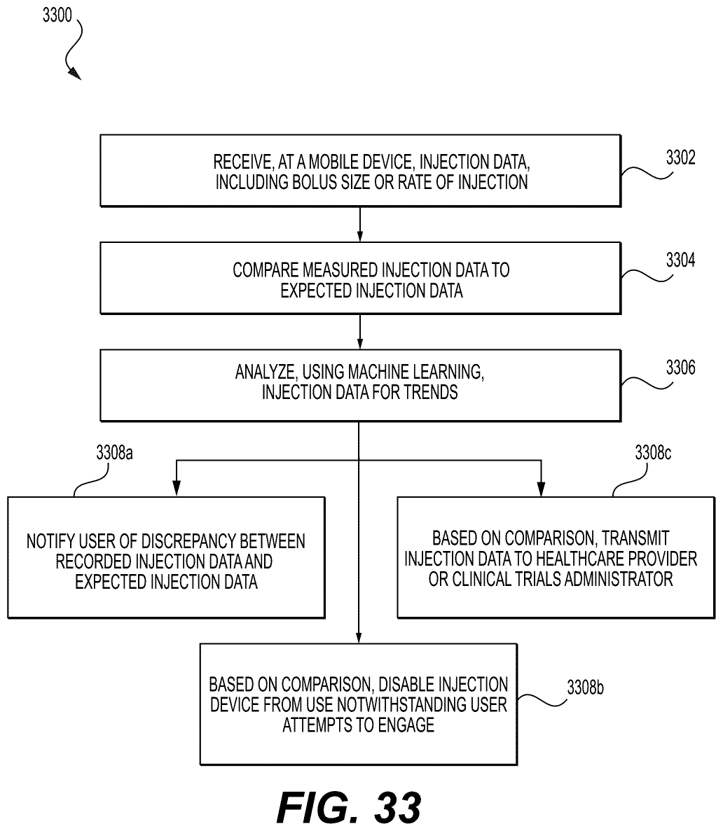

FIG. 33 shows an exemplary method 3300 of comparing injection data of auto-injector 2. Method 3300 may include step 3202, including receiving, at mobile device 1407, injection data from auto-injector 2. The injection data may include at least one of a bolus size and a rate of injection. Bolus size may be estimated by, e.g., the amount the piston 1316 is depressed within the cartridge 1302. Injection data may also include a needle length over time (e.g., needle 306 may dull or shorten with use). Method 3300 may include step 3304, including comparing the injection data to expected injection data. Step 3304 may include comparison methods described herein, e.g., direct comparison, statistical comparisons, etc.

Method 3300 may include optional step 3306, including analyzing the injection data for trends using a machine learning algorithm. Trends may be displayed on, e.g., mobile device 1407 for the user or patient to view.

Method 3300 may include steps 3308a-c, which are outcomes based on the comparison of the injection data. Step 3308a may include notifying a user of auto-injector 2 of a discrepancy between the injection data and the expected injection data. For example, if too little or too much medicament was administered, the user 1449 may be notified through audio feedback from the auto-injector 2 or through the mobile device 1407. Step 3308b may include disabling auto-injector 2 from use notwithstanding the user attempting to use auto-injector 2. Disabling auto-injector 2 may include controller 1455 of the mobile device 1407 disabling the needle 306 of the auto-injector 2 from being in the deployed configuration. Step 3308c may include transmitting the injection data, through wireless communications module 1415 of mobile device 1407, to an HCP 1451 or clinical trials administrator 1453.

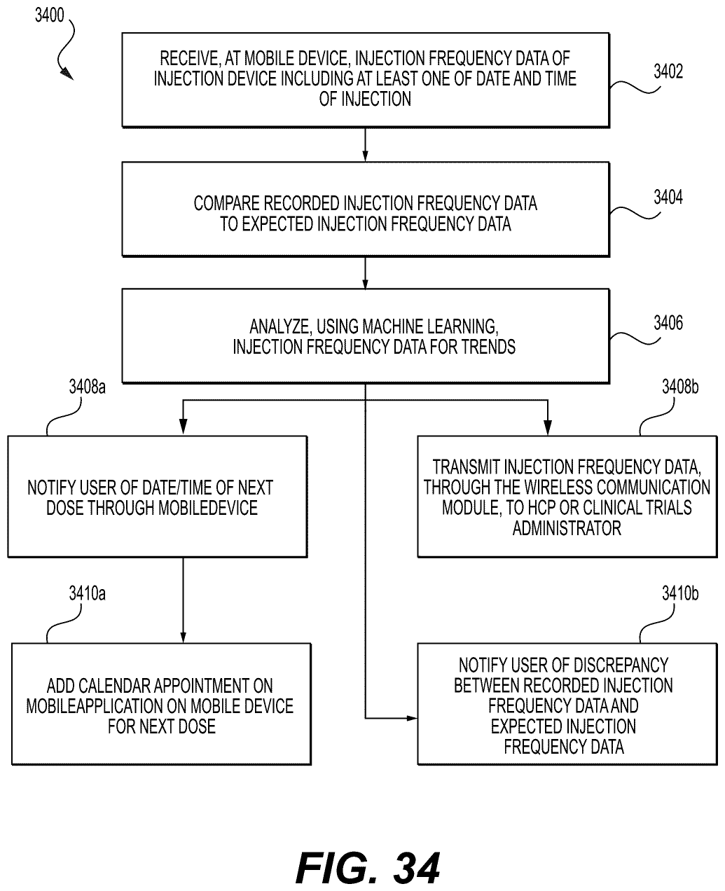

FIG. 34 shows an exemplary method 3400 of comparing injection frequency data. Method 3400 may include step 3402, including receiving injection data, at mobile device 1407, injection frequency data of the auto-injector 2. The auto-injector 2 may be a single-use (e.g., disposable) device. The injection frequency data may include at least one of a date of an injection and a time of an injection. The injection frequency data may rely on historical data of past injections that have been automatically recorded based on previous injections by the user 1449. For example, authentication may be required by the user 1449 to activate the auto-injector 2, then, upon successful completion of the injection, the auto injector 2 mobile application may automatically update the profile of the user 1449 to indicate a successful injection. There may be a prescribed time for the next injection, and the auto-injector 2 may be activated only within the prescribed window. Further, the auto-injector 2 may only be activated after a minimum time period has elapsed since a last injection (e.g., 24 hours).

Method 3400 may include step 3404, including comparing the injection frequency data to expected frequency data; and based on the comparison of the injection frequency data to expected frequency data, through the mobile device 1407. Step 3404 may include comparison methods described herein, e.g., direct comparison, statistical comparisons, etc. Expected injection frequency data may include how a drug administration regimen provided by, e.g., an HCP or manufacturer of the medicament. For example, the expected injection frequency of a medicament may be multiple times per day, daily, weekly, or any combination thereof. Method 3400 may include step 3408a, including notifying a user of the auto-injector 2 of the next dose through the mobile device 1407, e.g., notifying the user of a date and time of a subsequent expected injection. The notification may also or alternatively include a table of historical data including dates, times, injection sites, amount of medicament administered, and/or any other appropriate information. Method 3400 may further include an optional step 3410a including adding a calendar appointment on a mobile application of the mobile device 1407. In some implementations, such an appointment may be automatically schedule for the next expected injection on the mobile application on the mobile device 1407. In yet other implementations, the frequency data may be used to determine the time the patient 1447 is due to receive medicament administration. In such implementations, method 3400 may include notifying the user 1449 of the time to take the medicament. In yet another example, method 3400 may include notifying the user 1449 of a time to take the medicament out of refrigeration for warming prior to use.

Alternatively, or in addition, method 3400 may include step 3408b, including notifying the user of a discrepancy between the injection frequency data and the expected injection frequency data. The notification may include, for example, If, for example, the user administers the medicament outside of the expected injection frequency range, a notification may appear on the mobile device 1407 indicating that, e.g., too little or too much medicament was administered over a given period (e.g., day, week, or month). Method 3400 may include step 3408b, including transmitting the injection frequency data to at least one of an HCP and a clinical trials administrator.

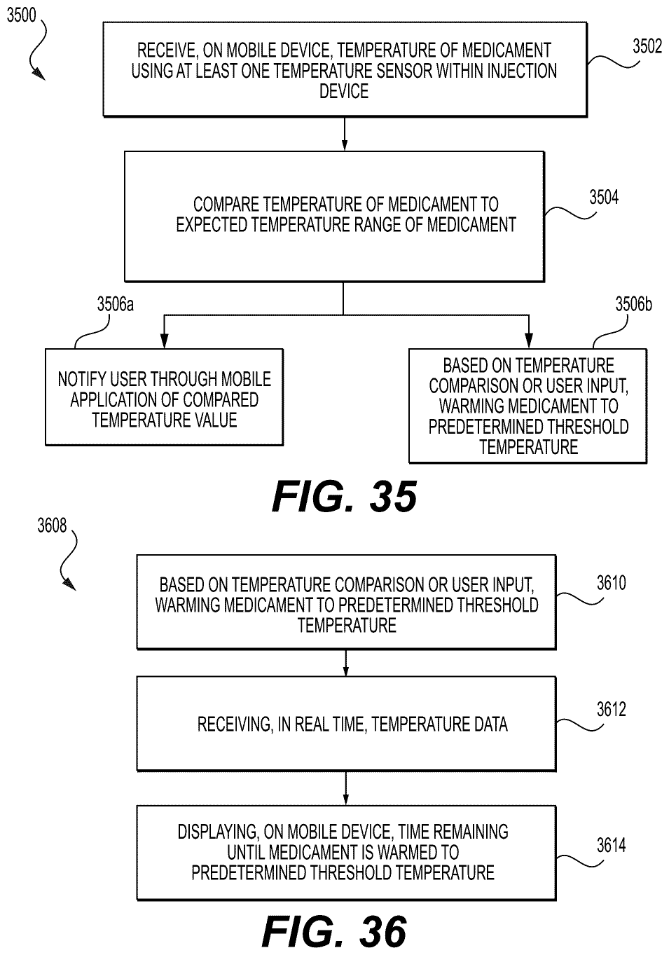

FIGS. 35 and 36 show exemplary methods 3500 and 3600 of comparing temperature data received from auto-injector 2. Method 3500 may include step 3502, including receiving temperature data of the auto-injector 2. The temperature data may be measured by at least one temperature sensor (e.g., thermometer 1437) coupled to the housing 3 of the auto-injector 2. Method 3500 may include step 3500 may include step 3504, comparing the temperature data to expected temperature data to determine if the medicament is at a predetermined threshold temperature. Step 3504 may include comparison methods described herein, e.g., direct comparison, statistical comparisons, etc.

Method 3500 may include a number of corrective actions, depending on the comparison in step 3504. In some implementations, when medicament has to be maintained below a threshold temp, e.g., a cold temperature, method 3500 may include continuously monitoring the temperature of the medicament. When the temperature approaches or passes a first threshold, which is still within predetermined temperature safe for administration, method 3500 may include sending a warning to the user 1449, HCP 1451, etc. If the temperature of the medicament passes a second threshold for greater than a threshold amount of time, the injector may be disabled. Method 3500 may include step 3506a, including, based on the comparison of the temperature data to the expected temperature data, notifying, through the mobile device 1407, a user of the auto-injector of the temperature data. Alternatively, or in addition, method 3500 may include step 3506b, including initiating a warming sequence of the medicament. Step 3506b may be automatic (e.g., without user intervention) or based on user input.

Referring to FIG. 36 now, method 3600 may include wherein warming the medicament includes warming the medicament with a heat element 1429 within the housing 3 of the auto-injector 2 and adjacent a cartridge 1302 or vial containing the medicament. Method 3600 may include step 3604, including displaying in real time, through the mobile device 1407, the temperature of the medicament as the medicament warms to the predetermined threshold temperature. The mobile device 1407 may further display an estimated time remaining until the medicament is warmed to the predetermined threshold temperature. In some implementations, method 3600 may further include disabling the auto-injector 2 based on a command from the mobile device 1407 transmitted to the controller 1408 of the auto-injector 2. The command may disable a medicament injection function of the auto-injector 2.

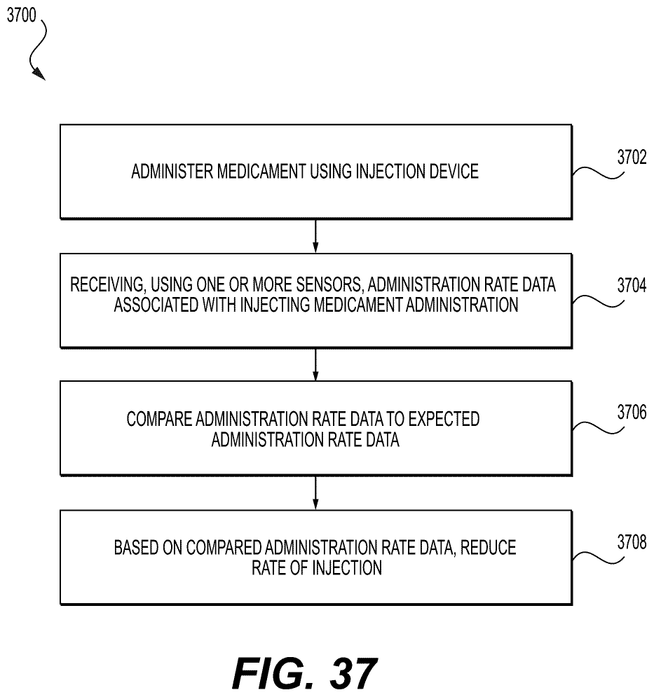

FIG. 37 shows an exemplary method 3700 of comparing administration rate data. Method 3700 may include optionally include step 3702 including administering a medicament with the auto-injector 2. Method 3700 may include step 3704, including receiving, during or after administering the medicament using the auto-injector 2, administration rate data from the auto-injector 2. Administration rate data may include the amount of medicament delivered to a patient per a unit of time (e.g., millisecond, seconds, minutes). Method 3700 may include step 3706, including comparing the administration rate data to expected administration rate data. Step 3704 may include comparison methods described herein, e.g., direct comparison, statistical comparisons, etc.

Method 3700 may include step 3708, including, based on the comparison of the administration rate data to the expected administration rate data, modifying the rate of injection of the medicament by sending a command from the mobile device 1407 to the auto-injector 2 that causes the motor in a piston 1316 of the auto-injector to change a rate the piston 1316 travels through the cartridge 1302 containing the medicament. Modification of the rate of injection may include reducing or increasing the rate of injection. Alternatively, if the administration rate data is within the expected range, take no action. In some implementations, method 3700 may including recommending to a user, using the mobile device 1407, a new injection site based on whether the administration rate data is below a threshold value of the expected administration rate data. For example, if the administration rate data is below a threshold value of the expected administration rate data, the controller 1408 of the auto-injector 2 may infer that the injection site is, e.g., too callused or swollen and may recommend to the user an alternative injection site.

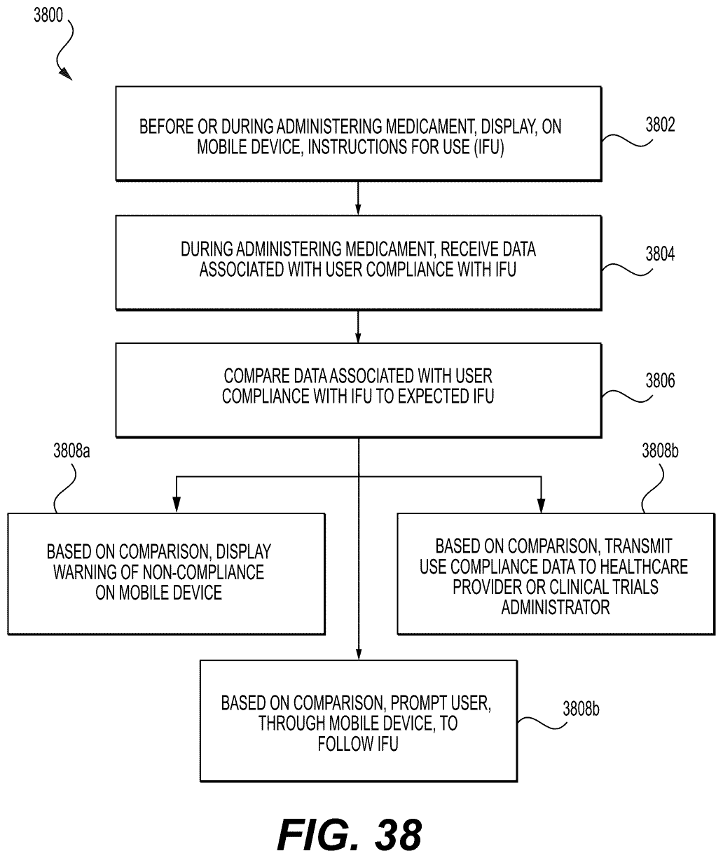

FIG. 38 shows an exemplary method 3800 of verifying patient compliance with instructions for use (IFU). Method 3800 may include step 3802, including displaying the IFU on e.g., the mobile device 1407. Method 3800 may include step 3804, including receiving, while administering the medicament to a user using the auto-injector 2, compliance data on the mobile device 1407. The compliance data may include user adherence to predetermined instructions for use associated with administering the medicament, e.g., an administration regimen provided by an HCP. Compliance data my include the frequency of injections, dates and times of injections, whether the patient 1447 was injected within a prescribed window of time or dates, and/or amount of medicament administered. Where appropriate, compliance data may be logged automatically and/or automatically sent to the HCP 1451 or clinical trials administrator 1453. Alternatively, method 3800 may include prompting a user 1449 to input in the mobile device 1407 whether the patient 1447 adhered to the medicament administration regimen.

Method 3800 may include step 3806, including comparing the compliance data with the instructions for use. Step 3304 may include comparison methods described herein, e.g., direct comparison, statistical comparisons, etc. Method 3800 may include steps 3808a-c, which are actions taken based on the comparison of the compliance data with the instructions for use. Step 3808a may including displaying, on the mobile device 1407, a warning of non-compliance. Step 3808b may include displaying, while administering a medicament to a user using an auto-injector, the IFU on a display of the mobile device 1407 so the patient and/or user may follow them. Alternatively, or in addition, step 3808c may include transmitting the compliance data to at least one of an HCP or clinical trials administrator. Alternatively, or in addition, method 3800 may include, based on the comparison, disabling the auto-injector 2 by sending a command from the controller 1455 of the mobile device 1407 disabling the needle 306 of the auto-injector 2 from being in a deployed configuration. For example, if too many injections are logged in a short amount of time, or if too high a dose of medicament is administered in too short amount of time, then the auto-injector 2 may be disabled to prevent toxicity in the patient 1447.

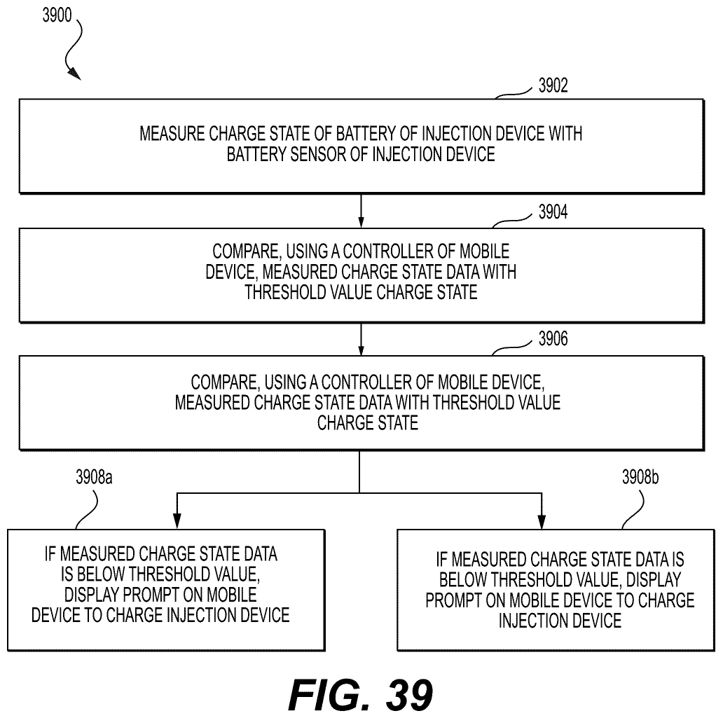

FIG. 39 shows an exemplary method 3900 comparing charge state of a battery of the auto-injector 2. Method 3900 may include step 3902, including receiving a charge state of the battery of the auto-injector 2. The charge state may be received in any appropriate manner, including through wired or wireless communication between the auto-injector and the mobile device 1407. The charge state may be measured by measuring the current into and out of the battery, the voltage of the battery, and/or the temperature of the battery.

Method 3900 may include step 3904, including comparing the charge state of the battery to a predetermined threshold value. Step 3904 may include comparison methods described herein, e.g., direct comparison, statistical comparisons, etc.

Method 3900 may include step 3906, including calculating the remaining battery life of the battery of the auto-injector 2. The remaining battery life of the battery may include the Coulomb counting, open circuit voltage methods, and/or using a Kalman filter.

Method 3900 may including steps 3908a and 3908b, each including an action taken based on the comparison in step 3906. Step 3908b may include displaying, if the charge state is less than the predetermined threshold value, a warning, on the mobile device, indicating that the charge state of the battery of the auto-injector is below the threshold value. This warning may include, e.g., instructions for plugging a charger into the auto-injector 2. Method 3900 may further include displaying, in real time, the charge state and an estimated time remaining until the charging is completed on, e.g., the mobile device 1407.

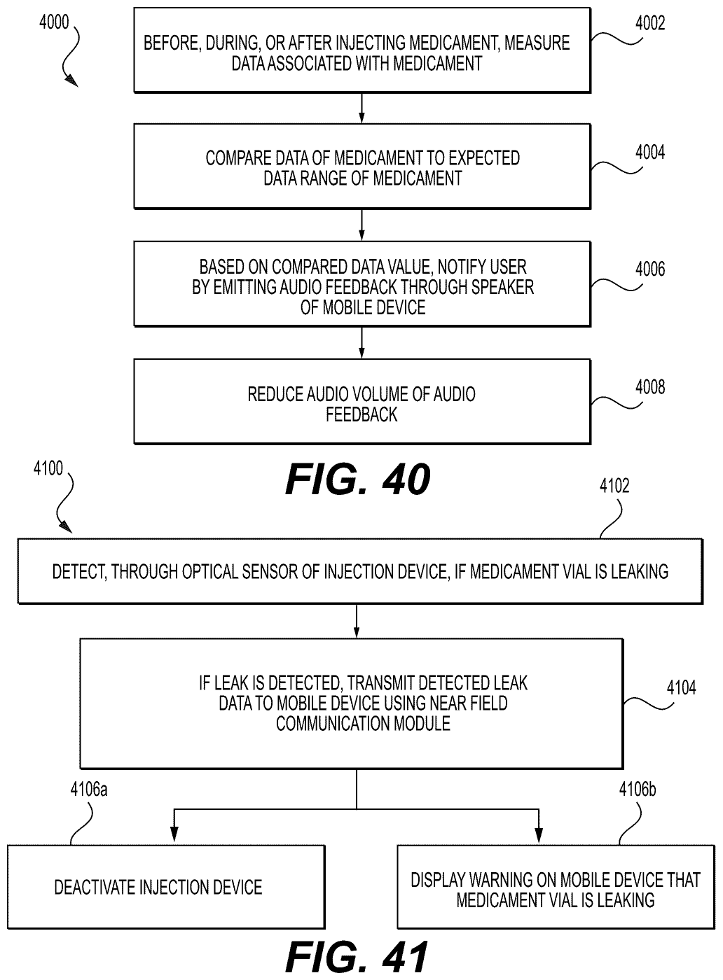

FIG. 40 shows an exemplary embodiment 4000 of emitting audio or flashing light feedback from the auto-injector 2. Method 4000 may include step 4002, including measuring data associated with the medicament. Data associated with the medicament may be any appropriate data, including an expiration date, lot information including lot number or serialization via QR code including component IDs, a manufacturer, volume level of the medicament within cartridge 1302, success/fail of an injection, whether needle 306 is broken, or a temperature of the medicament. Step 4002 may be completed before, during, or after administration of the medicament. Method 4000 may include step 4004, including comparing the data associated with the medicament to an expected data range of the medicament. Method 4000 may include step 4006, including, based on the comparison in step 4004, emitting audio feedback through a speaker of the auto-injector 2 and/or flashing lights of LEDs 52. The audio or flashing light feedback may be emitted for any appropriate reason, including an detecting an error state of the auto-injector 2, or for a successful/failed injection. In some implementations, the audio feedback may include a voice stating “injection complete,” or “injection failed.” Method 4000 may include an optional step 4008, including reducing the audio volume of the audio feedback or disabling the flashing lights

FIG. 41 shows an exemplary method 4100 of comparing detecting a leak in the auto-injector 2. Method 4100 may include step 4102, including detecting a leak of the medicament from a vial or cartridge 1302. A leak may be detected by an appropriate method including, e.g., an optical sensor of the auto-injector 2, pressure sensors, flow sensors, or temperature sensors. Leaks may be detected by detecting rapid temperature changes, chemical changes, etc. If a leak is detected, method 4100 may include step 4104, including transmitting the leak data to the mobile device 1407. Method 4100 may include steps 4106a and 4106, taking action in response to a detected leak. For example, step 4106a may include disabling the auto-injector 2 by disabling a needle 306 of the auto-injector 2 from being in a deployed configuration in response to detecting a leak. Step 4106b may include notifying a user that a leak has occurred within the auto-injector 2 by, e.g., displaying a warning through the mobile device 1407.

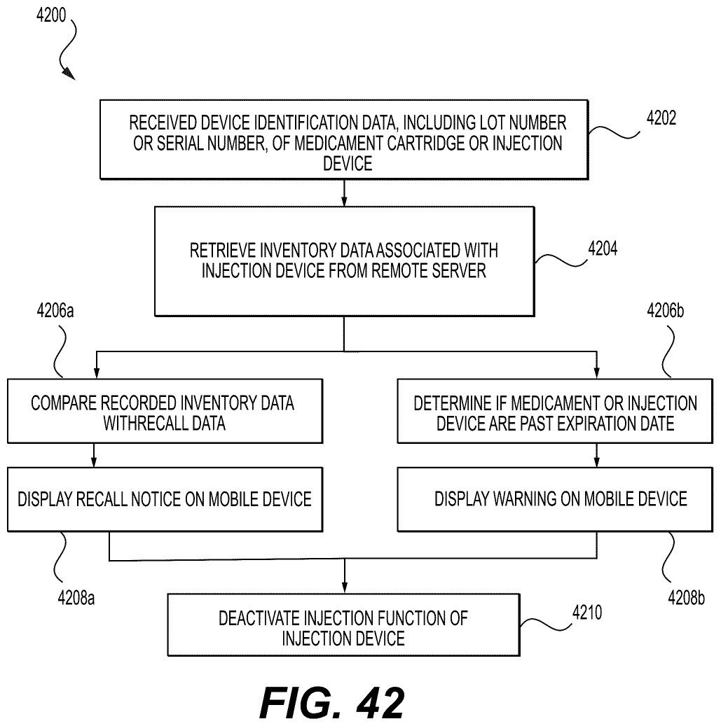

FIG. 42 shows an exemplary method 4200 of verifying the inventory history of the auto-injector 2 or the medicament. Method 4200 may include step 4202, including receiving device identification data associated with at least one of a medicament in the cartridge 1302 of the auto-injector 2, or the auto-injector 2 itself. The identification data may include any appropriate identifiers, including a serial number, batch number, a lot number or serialization via QR code including component IDs, manufacturer, medicament identifier (e.g., name), stock keeping unit (SKU), reorder point, or a bar code.

Method 4200 may include step 4204, including retrieving inventory data associated with the device identification data from a remote server. The associated inventory data may include updated information related to, e.g., the lot number or batch number, of the auto-injector 2 or cartridge 1302. For example, if there is a manufacturer recall on either the auto-injector 2 or cartridge 1302, then that information would be retrieved from the remote server. Step 4202 may be completed by, e.g., the mobile device 1407. Step 4202 may be completed automatically (e.g., without user input) or manually. In some implementations, method 4200 may include collecting inventory data on the personal inventory supply of the user 1449. In such instances, if the inventory data is below a threshold amount, method 4200 may include ordering and/or refilling the personal inventory of the user 1449. This may be done manually, by soliciting input from the user 1449 through a mobile application of the mobile device 1407, or automatically upon last dose. In other implementations, the retrieved inventory data may include the number of cartridges 1302 and/or auto-injectors 2 in stock at a given location (e.g., pharmacy, manufacturer warehouse, or healthcare provider's business location). When the inventory is low or completely depleted, method 4200 may include automated replenishment at a location where the inventory is depleted. For example, if the number of auto-injectors 2 and/or cartridges 1302 fall below a threshold value, method 4200 may include automatically sending a replenishment order to the manufacturer of the auto-injectors 2 and/or cartridges 1302. Inventory data may be used to determine sales data and/or market share in a given area. For example, based on how frequently stocks are depleted, the market share of the manufacturer of the auto-injector 2 and/or cartridge 1302 may be inferred.

Method 4200 may include steps 4206a and 4206b, which are comparative steps. Step 4206a includes comparing the received inventory data with recall data. If a recall has been issued on either the auto-injector 2 or the cartridge 1302, then method 4600 may proceed to step 4208a, which includes displaying a warning to the user on the mobile device 1407. Alternatively, or in addition, step 4202b may include comparing the received inventory data with expiration dates associated with the auto-injector 2 and/or cartridge 1302. If step 4202b determines that the auto-injector 2 and/or cartridge 1302 are past respective expiration dates, then method 4200 proceeds to step 4208b, including displaying a warning on the mobile device 1407. The warning may include, for example, a past expiration date and indication to discontinue using the medicament, or recall information, including date the recall was issued, the manufacturer, and the nature of the recall (e.g., contaminated medicament). In either situation, method 4200 may include step 4210, including, based on the comparison, disabling the needle 306 of the auto-injector 2 from being in a deployed configuration. Auto-injector 2 may be disabled via, e.g., a controller 1455 of the mobile device 1407 sending a command the controller 1408 of the auto-injector 2. Alternatively, or in addition, method 4200 may include purchasing more medicament and/or auto-injectors 2 through, e.g., the mobile device 1407 to replace the expired and/or recalled medicament or auto-injector 2.

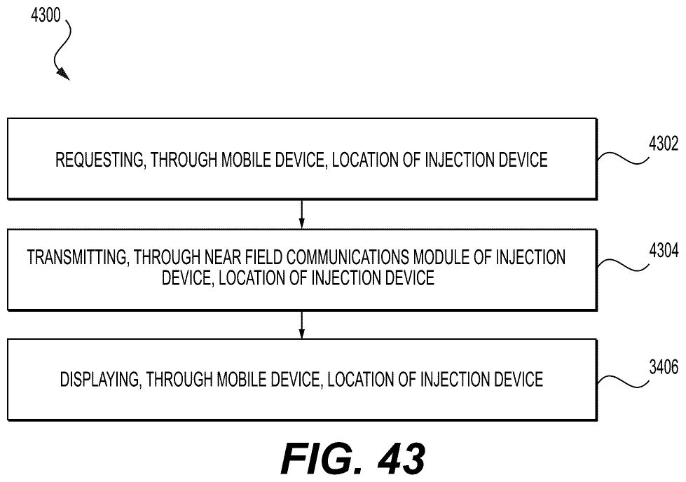

FIG. 43 illustrates an exemplary method 4300 of locating the auto-injector 2. Method 4300 may include step 4302, including requesting, through the mobile device 1407, a location of auto-injector 2. For example, if the auto-injector 2 is spatially remote from the user, the user may attempt to locate the auto-injector 2 through geolocation tracking. In some implementations, a user may track a location of the auto-injector 2 using indoor positioning systems (e.g., WiFi module 1419 and/or Bluetooth module 1457) and/or GPS module 1461. Location may also be determined by the user after enabling, remotely through the mobile device 1407, an audible feedback through the speaker of the auto-injector 2.

Method 4300 may include step 4304, including receiving a location signal associated with the auto-injector 2 and transmitted through a wireless communications module of the auto-injector 2. The auto-injector 2 may determine its own location using the wireless communication module 1411 thereof.

Method 4300 may include step 4306, including displaying, on a display of the mobile device 1407, the location of the auto-injector 2. Displaying the location may include, e.g., displaying a notification or map representation of the location of the auto-injector 2 relative to the mobile device 1407. The location may be displayed in real time or a table and/or map of historical locations may be displayed. In some implementations, the location data may be used to verify or predict the identity of the user 1449. For example, if the location of the auto-injector 2 is expected to be in one location, but the location data reveals that the auto-injector 2 is in another distant location, the mobile device 1407 may notify the user 1449 and/or disable the auto-injector 2. In other implementations, method 3400 may include disabling the auto-injector 2 if the auto-injector 2 is too far from the mobile device 1407. In another example, method 4300 may include disabling the auto-injector based on the proximity of the auto-injector 2 to, e.g., another auto-injector 2 to protect against accidental usage by the wrong user. Disabling the auto-injector 2 may be completed manually, through input by a user 1449 into the mobile device 1407, or automatically by the mobile device 1407. In such implementations, the controller 1408 of the auto-injector 2 may disable the needle 306 of the auto-injector 2 from being in a deployed configuration.

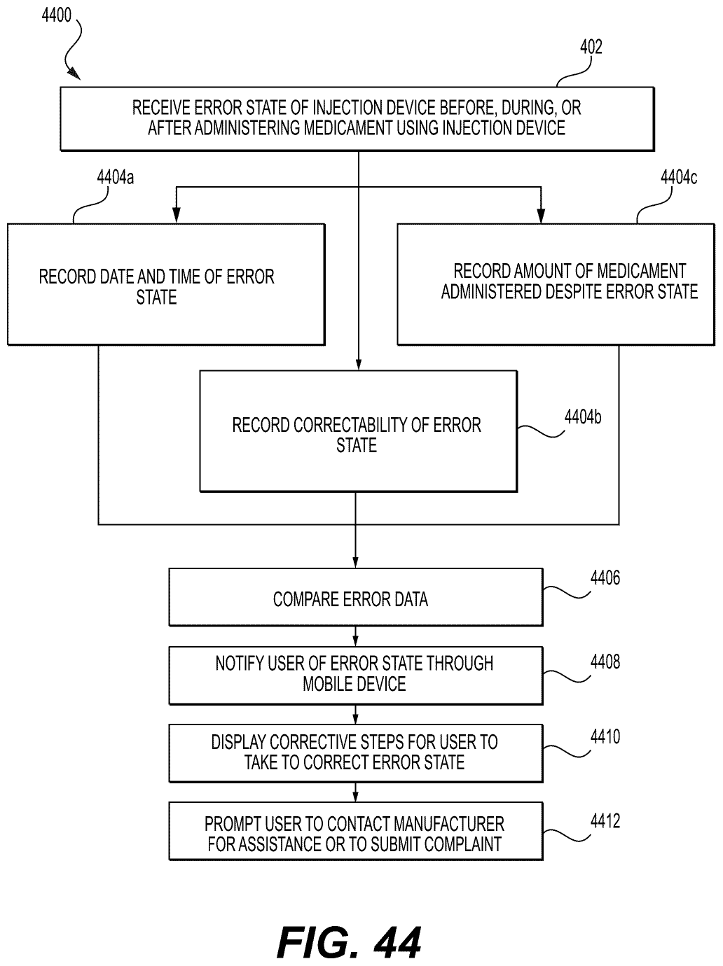

FIG. 44 shows an exemplary method 4400 for correcting an error state of the auto-injector 2. Method 4400 may include step 4402, including receiving, through the mobile device 1407, error data associated with an error state of the auto-injector 2. The error data may include at least one of type, a date, or a time associated with the error state, and an amount of medicament administered through the auto-injector to a user notwithstanding the error state. The type of error may be any type of failure, including needle deployment failures, mechanical failures, trigger mechanism failures, dosage failures (e.g., under- or over-dosing), medication type error, design flaws in the auto-injector 2, and/or battery failures. The error data may include whether an error of the auto-injector 2 includes complete or partial failure of the auto-injector 2 to administer the medicament to a user.

Method 4400 may include optional steps 4404a-c, which include step 4404a: recording the date and time error; 4404b: recording the correctability of the error state (e.g., whether a user correct the error state or if the error state is fatal to the auto-injector 2); and 4404c: recording the amount of medicament administered despite the error state.

Method 4400 may include step 4406, including comparing the error data with known error types to, e.g., determine the correctability of the error state, using any appropriate comparison methods described herein.

Method 4400 may include corrective steps 4408-4412. Step 4408 includes, based on the comparison of the error data with known error types, displaying a notification on the mobile device 1407 to inform the user of the error state. The notification may include the error data (e.g., type, date, or time of error) and/or instructions for corrective action. For example, the instructions for corrective action may include rebooting the auto-injector or re-injecting at a different injection site. If correctable, method 4400 may include step 4410, including displaying corrective steps for a user to take to correct the error state. This may be, e.g., a list of steps to follow to correct the error state. Method 4400 may include step 4412, including prompting the user to contact the manufacturer for assistance and/or to submit a complaint. If the error state is not correctable, method 4400 may include disabling, through the controller 1455 of the mobile device 1407, the needle 306 of the auto-injector 2 from being in a deployed configuration. Any complaint may be submitted through, e.g., a mobile application of the mobile device 1407.

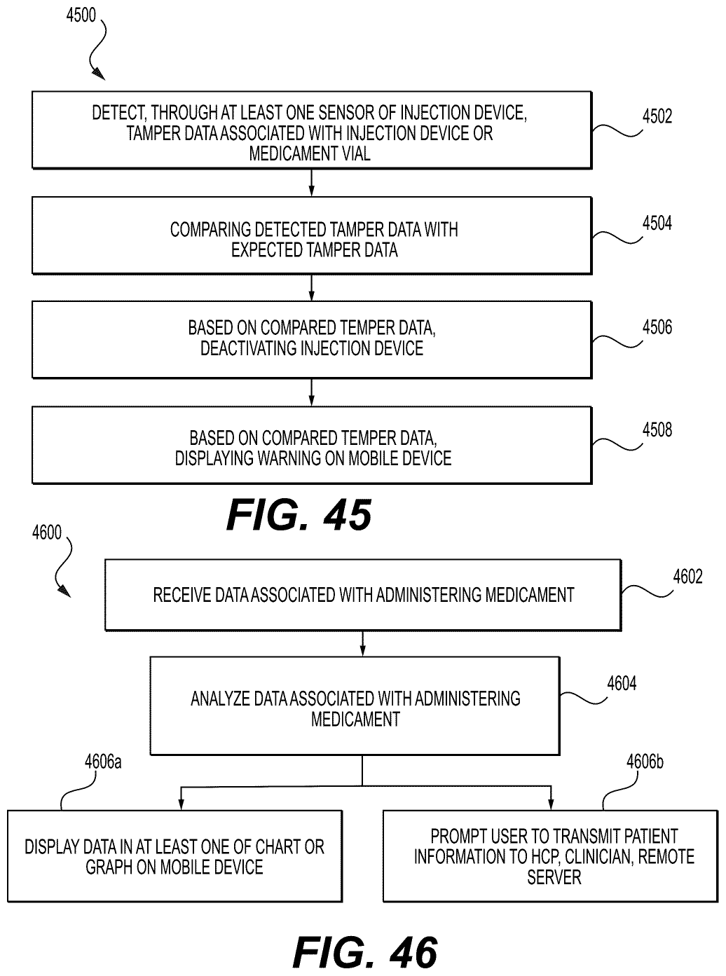

FIG. 45 shows an exemplary method 4500 for detecting whether the auto-injector 2 has been tampered with. Method 4500 may include step 4502, including detecting, using at least one sensors associated with the auto-injector 2, tamper data associated with auto-injector 2. Tamper data may be generated by tamper switches, e.g., physical switches that detect if the housing 3 of auto-injector 2 has been opened, optical tamper detection including light sensors to detect if the auto-injector 2 has been opened, integrated circuits designed to detect unauthorized access to the auto-injector 2, accelerometers 1435 and/or gyroscopes 1431 that detect motion or orientation changes, magnetic sensors that detect changes in magnetic fields. For example, detecting tamper data associated with the auto-injector 2 may include determining whether the housing 3 has been opened. In another example, method 4500 may include verifying whether a medicament of the auto-injector 2 has been tampered with.

Method 4500 may include step 4504 including comparing the detected tamper data with expected tamper data. The expected tamper data my include a desired state where the auto-injector 2 has not been tampered with. The expected tamper data may also include known tamper types, such as a state where the housing 3 has been opened. If tampering is identified, method 4500 may include disabling, through the controller 1455 of the mobile device 1407, the needle 306 of the auto-injector 2 from being in a deployed configuration.

FIG. 46 shows an exemplary method 4600 of displaying data associated with data collected through the auto-injector 2. Method 4600 may include step 4602, including receiving data associated with the auto-injector 2 and/or administering the medicament. The data associated with the auto-injector 2 may be any data, including the data described herein (e.g., temperature data, error state, charge state, etc.) Method 4600 may include step 4604, including analyzing the data associated with the auto-injector. Method 4600 may include steps 4606a-b, which take action on the analyzed data. For instance, in step 4606a, the analyzed data in one of a chart, graph, spreadsheet, etc. on a mobile device 1407. Alternatively, or in addition, step 4606b may include prompting the user to transmit the information to an HCP or clinical trials administrator.

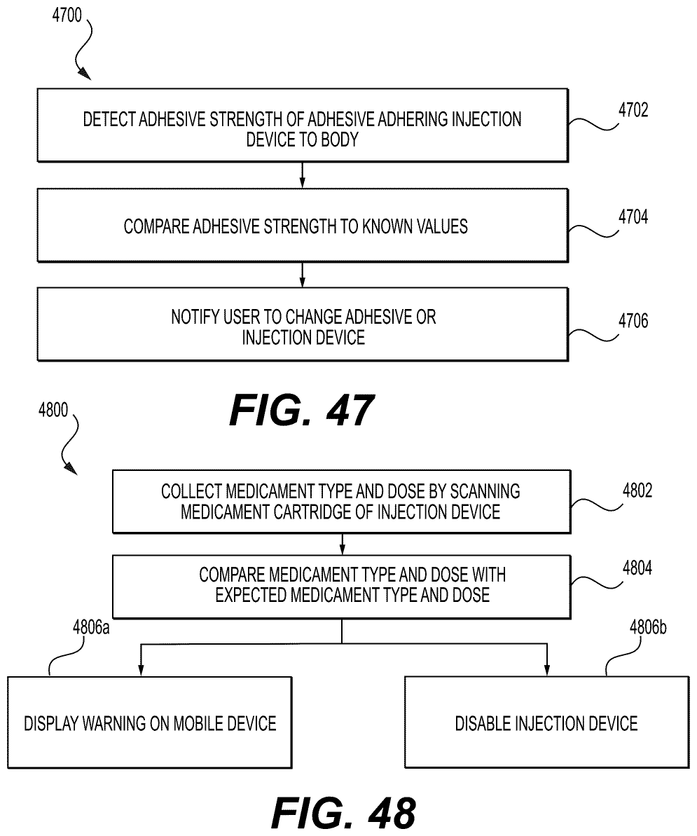

FIG. 47 shows an exemplary method 4700 of detecting the strength of an adhesive associated with the auto-injector 2, e.g. the adhesive patch 2. Method 4700 may include step 4702, including detecting the adhesive strength of an adhesive of the auto-injector 2. Step 4704 may include comparing the adhesive strength to known values, while step 4706 may include notifying the user, through the mobile device 1407, of, e.g., an adhesive force, a table of historical adhesive forces, and/or a recommendation, with instructions, to change the adhesive of the auto-injector 2.

FIG. 48 shows an exemplary method 4800 of determining the medicament type and dose of the medicament in the auto-injector 2. Method 4800 may include step 4802, including determining the medicament type and dose through scanning an identifier on the cartridge 1302 of the auto-injector 2. Scanning may be accomplished with any third party camera, e.g., a camera integrated in the mobile device 1407. Step 4804 includes comparing the medicament type and dose with the expected medicament type and dose for the patient that the auto-injector 2 is associated with. Based on the comparison in step 4802, method 4800 may proceed with steps 4806a or 4806b. For example, if the medicament type is not the expected medicament type, or the dose is the incorrect dose, for a given patient, then method 4806a proceeds with displaying a warning on the mobile device 1407 associated with the auto-injector 2, and/or with step 4806b, including disabling the auto-injector 2

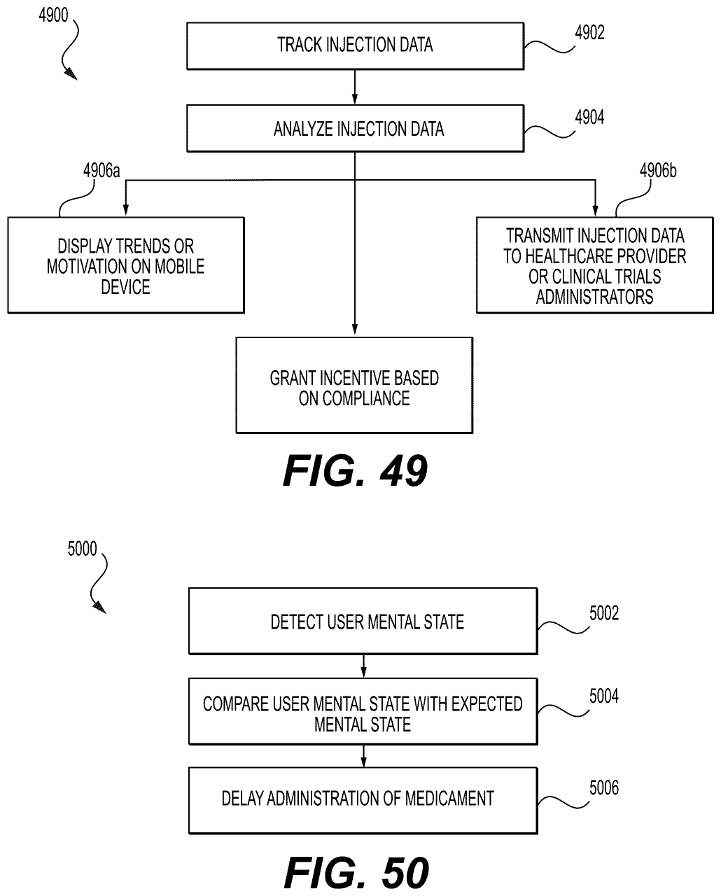

FIG. 49 shows an exemplary method 4900 of incentivizing compliance with a medicament administration regimen. A medicament administration regimen may be prescribed by an HCP along with the medicament to be delivered by the auto-injector 2. According, method 4900 may include step 4902, including tracking injection data associated with the auto-injector 2. Data may be tracked via, e.g., a mobile application of the mobile device 1407. Method 4904 may include analyzing the injection data for trends over a period of time. For example, data may be analyzed for daily injections in compliance with a prescription from an HCP. Depending on the data and trend analysis, the method 4900 may proceed with steps 4906a-c. Step 4906a includes displaying the trends and/or a motivational quote on the mobile device 1407 to incentivize compliance with the medicament administration regimen. Injection compliance may be gamified. If, based on the trend analysis, the patient is in compliance with the medicament administration regimen, the mobile application of the mobile device 1407 may grant an incentive to the patient. In other implementations, there may be an online leaderboard and/or posting of injection statistics. The incentive may include, for example, monetary rewards, cryptocurrency, discounts on future medicament purchases, and/or a digital badge. Alternatively, or in addition, step 4906c may include transmitting the injection data to an HCP or a clinical trials administrator for review or placement in the patient's medical file.

FIG. 50 shows an exemplary method 5000 of responding to a patient's mental state associated with administering the medicament with the auto-injector 2. Method 5000 may include step 5002, including detecting a mental state of the patient. This may include inputting a mental state of the patient into a mobile application of the mobile device 1407. Mental states may include, e.g., happy, sad, worried, nervous, anxious, etc. Alternatively, or in addition, the mental state may be an arbitrary scale of 1 to 10, based on the patient's apprehension to receiving the medicament. Method 5000 may include step 5004, including comparing the mental state of the patient with an expected mental state or an expected range of acceptable mental states. If the mental state of the patient is outside of the expected range, step 5006 may include delaying administration of the medicament to, e.g., allow the patient to calm down.

It should be understood that steps of one or more of the various methods described herein may be combined in certain embodiments. Furthermore, in certain embodiments, fewer than all of the steps of a method described herein may be performed and/or additional steps not described herein may be performed. Moreover, the steps described herein need not necessarily be performed in the exact order presented.

Notably, reference herein to “one embodiment,” or “an embodiment” means that a particular feature, structure, or characteristic described in connection with the embodiment may be included, employed and/or incorporated in one, some or all of the embodiments of the present disclosure. The usages or appearances of the phrase “in one embodiment” or “in another embodiment” in the specification are not referring to the same embodiment, nor are separate or alternative embodiments necessarily mutually exclusive of one or more other embodiments, nor limited to a single exclusive embodiment. The same applies to the terms “implementation,” and “example.” The present disclosure are neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

Further, as indicated above, an embodiment or implementation described herein as “exemplary” is not to be construed as preferred or advantageous, for example, over other embodiments or implementations; rather, it is intended convey or indicate the embodiment or embodiments are example embodiment(s).

The present disclosure is further described by the following non-limiting items:
Item 1. An auto-injector, comprising: a housing; a cartridge disposed within the housing, the cartridge enclosing a medicament; a fluid conduit configured to deliver the medicament from the cartridge to a patient and movable from a retracted configuration to a deployed configuration associated with administering the medicament; a temperature sensor configured to determine a temperature of the medicament; and a controller coupled to the temperature sensor, wherein the controller is configured to: send a first signal to an external device when the temperature sensor senses that a temperature of the medicament rises above a first threshold temperature.
Item 2. The auto-injector of claim 1, wherein the first threshold temperature is below a minimum safe temperature for the medicament.
Item 3. The auto-injector of claim 1, wherein the first signal causes a warning to display on the external device.
Item 4. The auto-injector of claim 3, wherein the controller is configured to send a second signal when the temperature of the medicament falls below the threshold temperature after rising above the threshold temperature.
Item 5. The auto-injector of claim 4, wherein the second signal is a retraction of the warning on the external device.
Item 6. The auto-injector of claim 1, wherein the controller is configured to prevent initiation of an injection by the auto-injector when the temperature of the medicament rises above a second threshold temperature that is higher than the first threshold temperature.
Item 7. The auto-injector of claim 6, wherein the second threshold temperature is a minimum safe temperature of the medicament.
Item 8. The auto-injector of claim 7, further including: a plunger coupled to the housing and movable relative to the housing; one or more electronics components used during an injection performed by the auto-injector, the one or more electronics components being formed within an electrical circuit, wherein: in a first configuration, a first portion of the plunger is disposed within the housing, the electrical circuit is open, and the one or more electronics components are in a low-power sleep mode; in a second configuration, the plunger moves outward relative to the housing, and the first portion of the plunger extends exterior of the housing; and in the second configuration, the electrical circuit is closed, and the one or more electronics components are transitioned from the low-power sleep mode, to an active mode; the plunger is movable from the second configuration toward the housing to a third configuration; and in the absence of a separate instruction from the controller, the auto-injector is configured to initiate an injection by the auto-injector only after the plunger is moved to the third configuration and the one or more electronics components are in the active mode; when the medicament rises above the second threshold temperature, the auto-injector configured to prevent initiation of an injection by the auto-injector after the plunger is moved to the third configuration.
Item 9. The auto-injector of claim 1, wherein the controller is configured to continuously transmit temperature data of the medicament to the external device.
Item 10. The auto-injector of claim 1, further comprising a heat element connected to the controller and adjacent the cartridge, the controller configured to selectively raise a temperature of the medicament through the heat element and based on an input provided by a user through a mobile device in communication with the auto-injector.
Item 11. An auto-injector, comprising: a cartridge disposed within a housing, the cartridge enclosing a medicament; a fluid conduit configured to deliver the medicament from the cartridge to a patient and movable from a retracted configuration to a deployed configuration; a location sensor; and a controller coupled to the location sensor, wherein the controller is configured to prevent initiation of an injection by the auto-injector based on a location detected by the location sensor.
Item 12. The auto-injector of claim 11, wherein the sensor includes at least one of a global positioning system (GPS) module, a WiFi module, a cellular modem, and a Bluetooth module.
Item 13. The auto-injector of claim 11, further including: a plunger coupled to the housing and movable relative to the housing; one or more electronics components used during an injection performed by the auto-injector, the one or more electronics components being formed within an electrical circuit, wherein: in a first configuration, a first portion of the plunger is disposed within the housing, the electrical circuit is open, and the one or more electronics components are in a low-power sleep mode; a second configuration, the plunger moves outward relative to the housing, and the first portion of the plunger extends exterior of the housing; and in the second configuration, the electrical circuit is closed, and the one or more electronics components are transitioned from the low-power sleep mode, to an active mode; the plunger is movable from the second configuration toward the housing to a third configuration; and in the absence of a separate instruction from the controller, the auto-injector is configured to initiate an injection by the auto-injector only after the plunger is moved to the third configuration and the one or more electronics components are in the active mode; the auto-injector being configured to prevent initiation of an injection by the auto-injector after the plunger is moved to the third configuration based on the location detected by the location sensor.
Item 14. The auto-injector of claim 11, further including a feedback module including at least one of an audio component configured to emit audible feedback, a visual component configured to emit visual feedback, and a haptic component configured to emit haptic feedback.
Item 15. The auto-injector of claim 14, wherein the controller is further configured to: receive a first signal from an external device; and cause, in response to the first signal, the feedback module to emit at least one of the audible feedback, the visual feedback, and the haptic feedback.
Item 16. The auto-injector of claim 14, wherein the audible feedback, when emitted, is configured to be reduced by a user through an external device.
Item 17. An auto-injector, comprising: a housing; a cartridge disposed within the housing, the cartridge enclosing a medicament; a fluid conduit configured to deliver the medicament from the cartridge to a patient and movable from a retracted configuration to a deployed configuration associated with administering the medicament; a fluid sensor configured to detect a fluid within the housing; and a controller, wherein the controller is configured to prevent initiation of an injection by the auto-injector when fluid sensor detects a fluid is detected within the housing.
Item 18. The auto-injector of claim 17, wherein the fluid sensor includes an optical fluid sensor.
Item 19. The auto-injector of claim 17, wherein the fluid sensor is adjacent a septum of the cartridge.
Item 20. The auto-injector of claim 17, further including: a plunger coupled to the housing and movable relative to the housing; one or more electronics components used during an injection performed by the auto-injector, the one or more electronics components being formed within an electrical circuit, wherein: in a first configuration, a first portion of the plunger is disposed within the housing, the electrical circuit is open, and the one or more electronics components are in a low-power sleep mode; in a second configuration, the plunger moves outward relative to the housing, and the first portion of the plunger extends exterior of the housing; and in the second configuration, the electrical circuit is closed, and the one or more electronics components are transitioned from the low-power sleep mode, to an active mode; the plunger is movable from the second configuration toward the housing to a third configuration; and in the absence of a separate instruction from the controller, the auto-injector is configured to initiate an injection by the auto-injector only after the plunger is moved to the third configuration and the one or more electronics components are in the active mode; when the fluid sensor detects a leak within the housing, the auto-injector is configured to prevent initiation of an injection by the auto-injector after the plunger is moved to the third configuration.

What is claimed is:

1. An auto-injector, comprising:
a housing;
a cartridge disposed within the housing, the cartridge enclosing a medicament;
a fluid conduit configured to deliver the medicament from the cartridge to a patient and movable from a retracted configuration to a deployed configuration associated with administering the medicament;
a temperature sensor configured to determine a temperature of the medicament; and
a controller coupled to the temperature sensor, wherein the controller is configured to:
send a first signal to an external device when the temperature sensor senses that a temperature of the medicament rises above a first threshold temperature.

2. The auto-injector of claim 1, wherein the first threshold temperature is below a minimum safe temperature for the medicament.

3. The auto-injector of claim 1, wherein the first signal causes a warning to display on the external device.

4. The auto-injector of claim 3, wherein the controller is configured to send a second signal when the temperature of the medicament falls below the threshold temperature after rising above the threshold temperature.

5. The auto-injector of claim 4, wherein the second signal is a retraction of the warning on the external device.

6. The auto-injector of claim 1, wherein the controller is configured to prevent initiation of an injection by the auto-injector when the temperature of the medicament rises above a second threshold temperature that is higher than the first threshold temperature.

7. The auto-injector of claim 6, wherein the second threshold temperature is a minimum safe temperature of the medicament.

8. The auto-injector of claim 7, further including:
a plunger coupled to the housing and movable relative to the housing;
one or more electronics components used during an injection performed by the auto-injector, the one or more electronics components being formed within an electrical circuit, wherein:
in a first configuration, a first portion of the plunger is disposed within the housing, the electrical circuit is open, and the one or more electronics components are in a low-power sleep mode;
in a second configuration, the plunger moves outward relative to the housing, and the first portion of the plunger extends exterior of the housing; and
in the second configuration, the electrical circuit is closed, and the one or more electronics components are transitioned from the low-power sleep mode, to an active mode;
the plunger is movable from the second configuration toward the housing to a third configuration; and
in the absence of a separate instruction from the controller, the auto-injector is configured to initiate an injection by the auto-injector only after the plunger is moved to the third configuration and the one or more electronics components are in the active mode;
when the medicament rises above the second threshold temperature, the auto-injector configured to prevent initiation of an injection by the auto-injector after the plunger is moved to the third configuration.

9. The auto-injector of claim 1, wherein the controller is configured to continuously transmit temperature data of the medicament to the external device.

10. The auto-injector of claim 1, further comprising a heat element connected to the controller and adjacent the cartridge, the controller configured to selectively raise a temperature of the medicament through the heat element and based on an input provided by a user through a mobile device in communication with the auto-injector.

11. An auto-injector, comprising:
a cartridge disposed within a housing, the cartridge enclosing a medicament;
a fluid conduit configured to deliver the medicament from the cartridge to a patient and movable from a retracted configuration to a deployed configuration;
a location sensor; and
a controller coupled to the location sensor, wherein the controller is configured to prevent initiation of an injection by the auto-injector based on a location detected by the location sensor.

12. The auto-injector of claim 11, wherein the sensor includes at least one of a global positioning system (GPS) module, a WiFi module, a cellular modem, and a Bluetooth module.

13. The auto-injector of claim 11, further including:
a plunger coupled to the housing and movable relative to the housing;
one or more electronics components used during an injection performed by the auto-injector, the one or more electronics components being formed within an electrical circuit, wherein:
in a first configuration, a first portion of the plunger is disposed within the housing, the electrical circuit is open, and the one or more electronics components are in a low-power sleep mode;
in a second configuration, the plunger moves outward relative to the housing, and the first portion of the plunger extends exterior of the housing; and
in the second configuration, the electrical circuit is closed, and the one or more electronics components are transitioned from the low-power sleep mode, to an active mode;
the plunger is movable from the second configuration toward the housing to a third configuration; and
in the absence of a separate instruction from the controller, the auto-injector is configured to initiate an injection by the auto-injector only after the plunger is moved to the third configuration and the one or more electronics components are in the active mode;
the auto-injector being configured to prevent initiation of an injection by the auto-injector after the plunger is moved to the third configuration based on the location detected by the location sensor.

14. The auto-injector of claim 11, further including a feedback module including at least one of an audio component configured to emit audible feedback, a visual component configured to emit visual feedback, and a haptic component configured to emit haptic feedback.

15. The auto-injector of claim 14, wherein the controller is further configured to:
receive a first signal from an external device; and
cause, in response to the first signal, the feedback module to emit at least one of the audible feedback, the visual feedback, and the haptic feedback.

16. The auto-injector of claim 14, wherein the audible feedback, when emitted, is configured to be reduced by a user through an external device.

17. An auto-injector, comprising:
a housing;
a cartridge disposed within the housing, the cartridge enclosing a medicament;
a fluid conduit configured to deliver the medicament from the cartridge to a patient and movable from a retracted configuration to a deployed configuration associated with administering the medicament;
a fluid sensor configured to detect a fluid within the housing; and
a controller, wherein the controller is configured to prevent initiation of an injection by the auto-injector when fluid sensor detects a fluid is detected within the housing.

18. The auto-injector of claim 17, wherein the fluid sensor includes an optical fluid sensor.

19. The auto-injector of claim 17, wherein the fluid sensor is adjacent a septum of the cartridge.

20. The auto-injector of claim 17, further including:
a plunger coupled to the housing and movable relative to the housing;
one or more electronics components used during an injection performed by the auto-injector, the one or more electronics components being formed within an electrical circuit, wherein:
in a first configuration, a first portion of the plunger is disposed within the housing, the electrical circuit is open, and the one or more electronics components are in a low-power sleep mode;
in a second configuration, the plunger moves outward relative to the housing, and the first portion of the plunger extends exterior of the housing; and
in the second configuration, the electrical circuit is closed, and the one or more electronics components are transitioned from the low-power sleep mode, to an active mode;
the plunger is movable from the second configuration toward the housing to a third configuration; and
in the absence of a separate instruction from the controller, the auto-injector is configured to initiate an injection by the auto-injector only after the plunger is moved to the third configuration and the one or more electronics components are in the active mode;
when the fluid sensor detects a leak within the housing, the auto-injector is configured to prevent initiation of an injection by the auto-injector after the plunger is moved to the third configuration.
